SEQUENCE LISTING (88 sequences)

SEQ ID NO: 1 - protein, 113 aa, synthetic construct [sequence omitted]

SEQ ID NO: 2 - protein, 107 aa, synthetic construct [sequence omitted]

SEQ ID NO: 3 - protein, 120 aa, synthetic construct [sequence omitted]

SEQ ID NO: 4 - protein, 111 aa, synthetic construct [sequence omitted]

SEQ ID NO: 5 - protein, 116 aa, synthetic construct
  source 1..116: note = dostarlimab VH
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSS

SEQ ID NO: 6 - protein, 107 aa, synthetic construct
  source 1..107: note = dostarlimab VL
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIK

SEQ ID NO: 7 - protein, 117 aa, synthetic construct [sequence omitted]

SEQ ID NO: 8 - protein, 107 aa, synthetic construct [sequence omitted]

SEQ ID NO: 9 - protein, 118 aa, synthetic construct [sequence omitted]

SEQ ID NO: 10 - protein, 107 aa, synthetic construct [sequence omitted]

SEQ ID NO: 11 - protein, 121 aa, synthetic construct
  source 1..121: note = durvalumab VH
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 12 - protein, 108 aa, synthetic construct
  source 1..108: note = durvalumab VL
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 13 - protein, 120 aa, synthetic construct
  source 1..120: note = avelumab VH
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 14 - protein, 110 aa, synthetic construct
  source 1..110: note = avelumab VL
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 15 - protein, 118 aa, synthetic construct
  source 1..118: note = ipilimumab VH
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 16 - protein, 108 aa, synthetic construct
  source 1..108: note = ipilimumab VL
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 17 - protein, 120 aa, synthetic construct
  source 1..120: note = relatlimab VH
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSS

SEQ ID NO: 18 - protein, 107 aa, synthetic construct
  source 1..107: note = relatlimab VL
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIK

SEQ ID NO: 19 - protein, 440 aa, synthetic construct
  source 1..440: note = nivolumab HC
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 214 aa, synthetic construct
  source 1..214: note = nivolumab LC
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 447 aa, synthetic construct
  source 1..447: note = pembrolizumab HC
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 218 aa, synthetic construct
  source 1..218: note = pembrolizumab LC
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 443 aa, synthetic construct
  source 1..443: note = dostarlimab HC
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 214 aa, synthetic construct
  source 1..214: note = dostarlimab LC
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 444 aa, synthetic construct
  source 1..444: note = cemiplimab HC
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 214 aa, synthetic construct
  source 1..214: note = cemiplimab LC
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 448 aa, synthetic construct
  source 1..448: note = atezolizumab HC
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 214 aa, synthetic construct
  source 1..214: note = atezolizumab LC
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 451 aa, synthetic construct
  source 1..451: note = durvalumab HC
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 215 aa, synthetic construct
  source 1..215: note = durvalumab LC
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 450 aa, synthetic construct
  source 1..450: note = avelumab HC
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 216 aa, synthetic construct
  source 1..216: note = avelumab LC
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 33 - protein, 448 aa, synthetic construct
  source 1..448: note = ipilimumab HC
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 215 aa, synthetic construct
  source 1..215: note = ipilimumab LC
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 446 aa, synthetic construct
  source 1..446: note = relatlimab HC
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 36 - protein, 214 aa, synthetic construct
  source 1..214: note = relatlimab LC
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 8 aa, synthetic construct
  source 1..8: note = nivolumab VH CDR1
GITFSNSG

SEQ ID NO: 38 - protein, 8 aa, synthetic construct
  source 1..8: note = nivolumab VH CDR2
IWYDGSKR

SEQ ID NO: 39 - protein, 6 aa, synthetic construct
  source 1..6: note = nivolumab VH CDR3
ATNDDY

SEQ ID NO: 40 - protein, 6 aa, synthetic construct
  source 1..6: note = nivolumab VL CDR1
QSVSSY

SEQ ID NO: 41 - protein, 9 aa, synthetic construct
  source 1..9: note = nivolumab VL CDR3
QQSSNWPRT

SEQ ID NO: 42 - protein, 8 aa, synthetic construct
  source 1..8: note = pembrolizumab VH CDR1
GYTFTNYY

SEQ ID NO: 43 - protein, 8 aa, synthetic construct
  source 1..8: note = pembrolizumab VH CDR2
INPSNGGT

SEQ ID NO: 44 - protein, 13 aa, synthetic construct
  source 1..13: note = pembrolizumab VH CDR3
ARRDYRFDMGFDY

SEQ ID NO: 45 - protein, 10 aa, synthetic construct
  source 1..10: note = pembrolizumab VL CDR1
KGVSTSGYSY

SEQ ID NO: 46 - protein, 9 aa, synthetic construct
  source 1..9: note = pembrolizumab VL CDR3
QHSRDLPLT

SEQ ID NO: 47 - protein, 8 aa, synthetic construct
  source 1..8: note = dostarlimab VH CDR1
GFTFSSYD

SEQ ID NO: 48 - protein, 8 aa, synthetic construct
  source 1..8: note = dostarlimab VH CDR2
ISGGGSYT

SEQ ID NO: 49 - protein, 9 aa, synthetic construct
  source 1..9: note = dostarlimab VH CDR3
ASPYYAMDY

SEQ ID NO: 50 - protein, 6 aa, synthetic construct
  source 1..6: note = dostarlimab VL CDR1
QDVGTA

SEQ ID NO: 51 - protein, 9 aa, synthetic construct
  source 1..9: note = dostarlimab VL CDR3
QHYSSYPWT

SEQ ID NO: 52 - protein, 8 aa, synthetic construct
  source 1..8: note = cemiplimab VH CDR1
GFTFSNFG

SEQ ID NO: 53 - protein, 8 aa, synthetic construct
  source 1..8: note = cemiplimab VH CDR2
ISGGGRDT

SEQ ID NO: 54 - protein, 10 aa, synthetic construct
  source 1..10: note = cemiplimab VH CDR3
VKWGNIYFDY

SEQ ID NO: 55 - protein, 6 aa, synthetic construct
  source 1..6: note = cemiplimab VL CDR1
LSINTF

SEQ ID NO: 56 - protein, 9 aa, synthetic construct
  source 1..9: note = cemiplimab VL CDR3
QQSSNTPFT

SEQ ID NO: 57 - protein, 8 aa, synthetic construct
  source 1..8: note = atezolizumab VH CDR1
GFTFSDSW

SEQ ID NO: 58 - protein, 8 aa, synthetic construct
  source 1..8: note = atezolizumab VH CDR2
ISPYGGST

SEQ ID NO: 59 - protein, 11 aa, synthetic construct
  source 1..11: note = atezolizumab VH CDR3
ARRHWPGGFDY

SEQ ID NO: 60 - protein, 6 aa, synthetic construct
  source 1..6: note = atezolizumab VL CDR1
QDVSTA

SEQ ID NO: 61 - protein, 9 aa, synthetic construct
  source 1..9: note = atezolizumab VL CDR3
QQYLYHPAT

SEQ ID NO: 62 - protein, 8 aa, synthetic construct
  source 1..8: note = durvalumab VH CDR1
GFTFSRYW

SEQ ID NO: 63 - protein, 8 aa, synthetic construct
  source 1..8: note = durvalumab VH CDR2
IKQDGSEK

SEQ ID NO: 64 - protein, 14 aa, synthetic construct
  source 1..14: note = durvalumab VH CDR3
AREGGWFGELAFDY

SEQ ID NO: 65 - protein, 7 aa, synthetic construct
  source 1..7: note = durvalumab VL CDR1
QRVSSSY

SEQ ID NO: 66 - protein, 9 aa, synthetic construct
  source 1..9: note = durvalumab VL CDR3
QQYGSLPWT

SEQ ID NO: 67 - protein, 8 aa, synthetic construct
  source 1..8: note = avelumab VH CDR1
GFTFSSYI

SEQ ID NO: 68 - protein, 8 aa, synthetic construct
  source 1..8: note = avelumab VH CDR2
IYPSGGIT

SEQ ID NO: 69 - protein, 13 aa, synthetic construct
  source 1..13: note = avelumab VH CDR3
ARIKLGTVTTVDY

SEQ ID NO: 70 - protein, 9 aa, synthetic construct
  source 1..9: note = avelumab VL CDR1
SSDVGGYNY

SEQ ID NO: 71 - protein, 10 aa, synthetic construct
  source 1..10: note = avelumab VL CDR3
SSYTSSSTRV

SEQ ID NO: 72 - protein, 8 aa, synthetic construct
  source 1..8: note = ipilimumab VH CDR1
GFTFSSYT

SEQ ID NO: 73 - protein, 8 aa, synthetic construct
  source 1..8: note = ipilimumab VH CDR2
ISYDGNNK

SEQ ID NO: 74 - protein, 11 aa, synthetic construct
  source 1..11: note = ipilimumab VH CDR3
ARTGWLGPFDY

SEQ ID NO: 75 - protein, 7 aa, synthetic construct
  source 1..7: note = ipilimumab VL CDR1
QSVGSSY

SEQ ID NO: 76 - protein, 9 aa, synthetic construct
  source 1..9: note = ipilimumab VL CDR3
QQYGSSPWT

SEQ ID NO: 77 - protein, 8 aa, synthetic construct
  source 1..8: note = relatlimab VH CDR1
GGSFSDYY

SEQ ID NO: 78 - protein, 7 aa, synthetic construct
  source 1..7: note = relatlimab VH CDR2
INHRGST

SEQ ID NO: 79 - protein, 14 aa, synthetic construct
  source 1..14: note = relatlimab VH CDR3
AFGYSDYEYNWFDP

SEQ ID NO: 80 - protein, 6 aa, synthetic construct
  source 1..6: note = relatlimab VL CDR1
QSISSY

SEQ ID NO: 81 - protein, 9 aa, synthetic construct
  source 1..9: note = relatlimab VL CDR3
QQRSNWPLT

SEQ ID NO: 82 - protein, 8 aa, synthetic construct
  source 1..8: note = LAG-3 eptitope
PGHPLAPG

SEQ ID NO: 83 - protein, 8 aa, synthetic construct
  source 1..8: note = LAG-3 eptitope
HPAAPSSW

SEQ ID NO: 84 - protein, 8 aa, synthetic construct
  source 1..8: note = LAG-3 eptitope
PAAPSSWG

SEQ ID NO: 85 - protein, 219 aa, Homo sapiens
  source 1..219: note = IL-12 p35
MGPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 86 - protein, 328 aa, Homo sapiens
  source 1..328: note = IL-12 p40
MGHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 87 - DNA, 660 bp, Homo sapiens
  source 1..660: mol_type = genomic DNA; note = IL-12 p35
atgggtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 88 - DNA, 989 bp, Homo sapiens
  source 1..989: mol_type = genomic DNA; note = IL-12 p35
atgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttagac

(19) United States
(12) Patent Application Publication
(10) Pub. No.: US 2026/0315624 A1
(43) Pub. Date: Oct. 15, 2026

(54) IL-12 GENE THERAPY AND IMMUNE CHECKPOINT INHIBITOR COMBINATION FOR TREATING CANCER

(71) Applicant: IMUNON, INC., Lawrenceville, NJ (US)

(72) Inventors: Khursheed ANWER, Madison, AL (US); Nicholas BORYS, New Hope, PA (US)

(21) Appl. No.: 19/111,091

(22) PCT Filed: Sep. 13, 2023
(86) PCT No.: PCT/US2023/074062

§ 371 Date: Mar. 12, 2025

Related U.S. Application Data
(60) Provisional application No. 63/375,529, filed on Sep. 13, 2022.

(51) Int. Cl. A61K 48/00; A61K 9/51; A61K 31/282; A61K 31/337; A61K 31/502; A61K 38/20; A61K 39/00; A61K 39/395; A61P 35/00

(52) U.S. Cl. CPC A61K 48/0041; A61K 9/5146; A61K 31/282; A61K 31/337; A61K 31/502; A61K 38/208; A61K 39/3955; A61K 48/005; A61K 48/0075; A61K 48/0083; A61P 35/00; A61K 2039/507

(57) ABSTRACT

The present disclosure relates to a combination therapy comprising an immune checkpoint inhibitor, a nanoparticle formulated plasmid comprising an IL-12 coding nucleic acid, and, optionally, at least one adjunctive chemotherapeutic drug, and methods of treatment using such combination therapies and/or compositions.

### CROSS-REFERENCE TO RELATED APPLICATIONS

The present application claims the priority benefit of U.S. Provisional Application 63/375,529, filed Sep. 13, 2022, which is hereby incorporated by reference in its entirety.

### REFERENCE TO SEQUENCE LISTING FILED ELECTRONICALLY

The content of the electronically submitted sequence listing in .XML file (Name: 2437_082PC01_Sequencelisting_ST26; Size: 102,830 bytes; and Date of Creation: Aug. 31, 2023), filed with the application, is incorporated herein by reference in its entirety.

### FIELD OF THE DISCLOSURE

The present disclosure relates to the fields of cancer therapy, gene therapy, and immunology.

### BACKGROUND

IL-12 is one of the most active cytokines for stimulating an immune response against cancer. However, when administered as a recombinant protein, the pharmacokinetics of IL-12 requires that it be administered by frequent, large bolus injections, resulting in serious toxicities that limit its use. The GEN-1 is an IL-12 DNA plasmid vector formulated using a lipopolymeric delivery system. GEN-1 can be delivered locally (e.g., intraperitoneally), offering the potential for cytokines to be expressed specifically in the tumor micro-environment with the goal of achieving increased efficacy while minimizing potential systemic toxicity.

While traditional chemotherapy regimens are designed to inhibit tumor growth by cytotoxic mechanisms, immunocytokine therapies are designed to elicit tumor killing by enhancing the immune system against cancer cells. GEN-1 reduces the toxicity issues associated with IL-12. Its nanoparticle profile allows for cell transfection followed by persistent, local secretion of IL-12 at therapeutic levels, while avoiding the toxicities associated with recombinant IL-12.

### BRIEF SUMMARY

Certain aspects of the disclosure are related to a combination therapy comprising: (i) a nucleic acid vector (e.g., a plasmid) comprising a polynucleotide that encodes an interleukin-12 (IL-12) formulated with a lipopolymer (e.g., a nanoparticle); and (ii) an immune checkpoint inhibitor.

Certain aspects of the disclosure are related to a method of treating a subject suffering from cancer comprising administering to the subject a combination therapy comprising: (i) a nucleic acid vector (e.g., a plasmid) comprising a polynucleotide that encodes an interleukin-12 (IL-12) formulated with a lipopolymer (e.g., a nanoparticle); and (ii) an immune checkpoint inhibitor.

In some aspects, the polynucleotide encodes human IL-12.

In some aspects, nucleic acid vector (e.g., a plasmid) comprises a promoter operably linked to a nucleic acid encoding a p35 subunit of IL-12 and a promoter operably linked to a nucleic acid encoding a p40 subunit of IL12.

In some aspects, the promoter is a CMV promoter.

In some aspects, nucleic acid vector (e.g., a plasmid) comprises an intron, a 3′ UTR (e.g., hGH 3′ UTR), an antibiotic resistance gene, or any combination thereof (e.g., the elements of FIG. 1).

In some aspects, the lipopolymer comprises a polyethyleneimine (PEI) covalently linked independently to cholesterol and polyethylene glycol (PEG) groups (e.g., the lipopolymer of FIG. 2).

In some aspects, the combination further comprises an anticancer agent.

In some aspects, the anticancer agent is a chemotherapeutic agent.

In some aspects, the chemotherapeutic agent is selected from a group consisting of topoisomerase inhibitors (e.g., irinotecan, topotecan, doxorubicin, epirubicin, idarubicin), anti-microtubule agents (e.g., paclitaxel, docetaxel), alkylating agents (e.g., cyclophosphamide, dacarbizine), platinum-based drugs (cisplatin, carboplatin, oxaliplatin), anti-metabolites (e.g., gemcitabine, methotrexate, 5-fluorouracil), or combinations thereof.

In some aspects, the chemotherapeutic agent is selected from the group consisting of doxorubicin, paclitaxel, carboplatin, docetaxel, nab-paclitaxel, olaparib, and any combination thereof.

In some aspects, the anticancer agent is doxorubicin.

In some aspects, the anticancer agent is paclitaxel.

In some aspects, the anticancer agent is carboplatin.

In some aspects, the anticancer agent is docetaxel.

In some aspects, the anticancer agent is nab-paclitaxel.

In some aspects, the anticancer agent is olaparib.

In some aspects, the immune checkpoint inhibitor is an inhibitor of an immune checkpoint protein selected from the group consisting of CTLA-4, PD-1 (and its ligands PD-L1 and PD-L2), and/or LAG-3.

In some aspects, the immune checkpoint inhibitor is an antibody.

In some aspects, the immune checkpoint inhibitor is a small molecule inhibitor.

In some aspects, the immune checkpoint inhibitor is a PD-1 antagonist selected from the group consisting of nivolumab, pembrolizumab, dostarlimab, and cemiplimab.

In some aspects, the immune checkpoint inhibitor is a PD-1 antagonist, wherein the PD-1 antagonist is nivolumab.

In some aspects, the immune checkpoint inhibitor is a PD-L1 antagonist selected from the group consisting of atezolizumab, durvalumab, and avelumab.

In some aspects, the immune checkpoint inhibitor is a CTLA-4 antagonist is ipilimumab.

In some aspects, the immune checkpoint inhibitor is a LAG-3 antagonist, wherein the LAG-3 antagonist is relatlimab.

In some aspects, the combination therapy further comprises (c) a second immune checkpoint inhibitor.

In some aspects, the second immune checkpoint inhibitor is an inhibitor of an immune checkpoint protein selected from the group consisting of CTLA-4, PD-1 (and its ligands PD-L1 and PD-L2), and/or LAG-3.

In some aspects, second the immune checkpoint inhibitor is an antibody.

In some aspects, the second immune checkpoint inhibitor is a small molecule inhibitor.

In some aspects, the second immune checkpoint inhibitor is a PD-1 antagonist selected from the group consisting of nivolumab, pembrolizumab, dostarlimab, and cemiplimab.

In some aspects, the second immune checkpoint inhibitor is nivolumab.

In some aspects, the second immune checkpoint inhibitor is a PD-L1 antagonist selected from the group consisting of atezolizumab, durvalumab, and avelumab.

In some aspects, the second immune checkpoint inhibitor is a CTLA-4 antagonist is ipilimumab.

In some aspects, the second immune checkpoint inhibitor is a LAG-3 antagonist, wherein the LAG-3 antagonist is relatlimab.

In some aspect, the method further comprises a surgery to remove all or part of a tissue or tumor (e.g., interval cytoreductive surgery) in the subject.

In some aspects, the nucleic acid vector formulated with the lipopolymer is administered intratumorally or intraperitoneally.

In some aspects, the nucleic acid vector formulated with the lipopolymer is administered intravenously.

In some aspects, the immune checkpoint inhibitor is administered intratumorally, intraperitoneally, intravenously, intravesicularly, or any combination thereof.

In some aspects, the immune checkpoint inhibitor is administered intratumorally or intraperitoneally.

In some aspects, the immune checkpoint inhibitor is administered intravenously.

In some aspects, the immune checkpoint inhibitor is administered intravesicularly.

In some aspects, the nucleic acid vector formulated with the lipopolymer is administered prior to, concurrently with, or after the immune checkpoint inhibitor

In some aspects, the nucleic acid vector formulated with the lipopolymer is administered prior to, concurrently with, or after the anticancer agent.

In some aspects, an anticancer agent is administered (e.g., first), followed by the administration of the nucleic acid vector formulated with the lipopolymer (e.g., second), and followed by administration of the immune checkpoint inhibitor (e.g., third).

In some aspects, an anticancer agent is administered (e.g., first), followed by the administration of the nucleic acid vector formulated with the lipopolymer (e.g., second), followed by the immune checkpoint inhibitor (e.g. third), and followed by a surgery to remove all or part of a tissue or tumor (e.g., interval cytoreductive surgery) (e.g., fourth).

In some aspects, a surgery to remove all or part of a tissue or tumor (e.g., interval cytoreductive surgery) is administered (e.g., first), followed by the administration of the nucleic acid vector formulated with the lipopolymer is administered (e.g., second), and followed by administration of an immune checkpoint inhibitor (e.g., third, fourth, etc. depending on how many immune checkpoint inhibitors are administered).

In some aspects, an anticancer agent is administered, followed by the administration of a nucleic acid vector comprising a polynucleotide encoding IL-12 (e.g., a DNA plasmid), followed by the immune checkpoint inhibitor, followed by an interval cytoreductive surgery.

In some aspects, the administration of the anticancer agent comprises administering paclitaxel at a dose of about 25-250 mg/m2, optionally, followed by administering carboplatin at a dose of about AUC 4-6 IV.

In some aspects, the administration of the anticancer agent comprises administering docetaxel at a dose of 25-250 mg/m2, optionally, followed by administering carboplatin at a dose of about AUC 4-6 IV.

In some aspects, the administration of the anticancer agent comprises administering nab-paclitaxel at a dose of 25-350 mg/m², optionally followed by administering carboplatin at a dose of about AUC 4-6 IV.

In some aspects, the interleukin-12 (IL-12) formulated with a lipopolymer (e.g., a nanoparticle) is administered at a dose of about 35 mg/m² to about 80 mg/m².

In some aspects, the cancer is selected from a group consisting of ovarian cancer, fallopian tube cancer, primary peritoneal cancer, cervical cancer, breast cancer, prostate cancer, colorectal cancer, bladder cancer, brain cancer (e.g., glioblastoma), lung cancer, and any combination thereof, and metastasis of any of the cancers.

In some aspects, the cancer is selected from a group consisting of ovarian cancer, fallopian tube cancer, primary peritoneal cancer, and any combination thereof.

In some aspects, the subject is a human.

In some aspects, the lipopolymer (e.g., nanoparticle) is administered at a dose of about 60 mg/m².

In some aspects, the immune checkpoint inhibitor is nivolumab, and wherein nivolumab is administered at about 240 mg.

In some aspects, nivolumab and the lipopolymer (e.g., nanoparticle) are administered every 1-4 weeks (e.g., every 2 weeks) for the duration of treatment.

In some aspects, the second inhibitor is ipilimumab, and wherein ipilimumab is administered at about 1 mg/kg.

In some aspects, ipilimumab is administered every 2-8 weeks (e.g., every 6 weeks) for the duration of treatment.

In some aspects, the nucleic acid vector is a plasmid.

In some aspects, the lipopolymer is a nanoparticle.

### DESCRIPTION OF FIGURES

FIG. 1 shows an exemplary human IL-12 (hIL-12) expression plasmid.

FIG. 2 shows the PEG-PEI-Cholesterol structure.

### DETAILED DESCRIPTION

### I. Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Throughout this disclosure, various aspects are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “effective amount” or “pharmaceutically effective amount” or “therapeutically effective amount” as used herein refers to the amount or quantity of a drug or pharmaceutically active substance which is sufficient to elicit the required or desired therapeutic response, or in other words, the amount which is sufficient to elicit an appreciable biological response when administered to a patient.

“Transfecting” or “transfection” shall mean transport of nucleic acids from the environment external to a cell to the internal cellular environment, with particular reference to the cytoplasm and/or cell nucleus. Without being bound by any particular theory, it is to be understood that nucleic acids may be delivered to cells either after being encapsulated within or adhering to one or more cationic polymer/nucleic acid complexes or being entrained therewith. Particular transfecting instances deliver a nucleic acid to a cell nucleus. Nucleic acids include DNA and RNA as well as synthetic congeners thereof. Such nucleic acids include missense, antisense, nonsense, as well as protein producing nucleotides, on and off and rate regulatory nucleotides that control protein, peptide, and nucleic acid production. In particular, but not limited to, they can be genomic DNA, cDNA, mRNA, tRNA, rRNA, hybrid sequences or synthetic or semi-synthetic sequences, and of natural or artificial origin. In addition, the nucleic acid can be variable in size, ranging from oligonucleotides to chromosomes. These nucleic acids may be of human, animal, vegetable, bacterial, viral, or synthetic origin. They may be obtained by any technique known to a person skilled in the art.

As used herein, the term “pharmaceutical agent” or “drug” or any other similar term means any chemical or biological material or compound suitable for administration by the methods previously known in the art and/or by the methods taught in the present disclosure, which induce a desired biological or pharmacological effect, which may include but are not limited to (1) having a prophylactic effect on the organism and preventing an undesired biological effect such as preventing an infection, (2) alleviating a condition caused by a disease, for example, alleviating pain or inflammation caused as a result of disease, and/or (3) either alleviating, reducing, or completely eliminating a disease from the organism. The effect may be local, such as providing for a local anesthetic effect, or it may be systemic.

As used herein, the term “biocompatible” or “biodegradation” is defined as the conversion of materials into less complex intermediates or end products by solubilization hydrolysis, or by the action of biologically formed entities which can be enzymes and other products of the organism.

As used herein, “effective amount” means the amount of a nucleic acid or a bioactive agent that is sufficient to provide the desired local or systemic effect and performance at a reasonable risk/benefit ratio as would attend any medical treatment.

As used herein, “peptide” means peptides of any length and includes proteins. The terms “polypeptide” and “oligopeptide” are used herein without any particular intended size limitation, unless a particular size is otherwise stated.

As used herein, a “derivative” of a carbohydrate includes, for example, an acid form of a sugar, e.g. glucuronic acid; an amine of a sugar, e.g. galactosamine; a phosphate of a sugar, e.g. mannose-6-phosphate and the like.

As used herein, “administering” and similar terms mean delivering the composition to the individual being treated such that the composition is capable of being circulated systemically where the composition binds to a target cell and is taken up by endocytosis. Thus, the composition is preferably administered systemically to the individual, typically by subcutaneous, intramuscular, transdermal, intravenous, or intraperitoneal routes. Injectables for such use can be prepared in conventional forms, either as a liquid solution or suspension, or in a solid form that is suitable for preparation as a solution or suspension in a liquid prior to injection, or as an emulsion. Suitable excipients that can be used for administration include, for example, water, saline, dextrose, glycerol, ethanol, and the like; and if desired, minor amounts of auxiliary substances such as wetting or emulsifying agents, buffers, and the like.

As used herein, “efficacy” and similar terms means disappearance of tumor or shrinkage of tumor in size or reduction in tumor density or increase in lymphocyte count or increase in neutrophil count or improvement in survival, or all of the above.

As used herein, “toxicity” is defined as any treatment related adverse effects on clinical observation including but not limited to abnormal hematology or serum chemistry results or organ toxicity.

As used herein, the term “promoter/regulatory sequence” refers to a nucleic acid sequence required to express a gene product operably linked to a promoter/regulatory sequence. The term “constitutive” promoter refers to a nucleotide sequence that, when operably linked to a polynucleotide encoding or specifying a gene product, results in the production of a gene product in the cell under most or all physiological conditions of the cell. The term “inducible” promoter means that when operably linked to a polynucleotide encoding a specified gene product, it basically results in the production of a gene in the cell only when the inducer corresponding to the promoter is present in the cell the nucleotide sequence of the product.

As used herein, the term “expression” refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into messenger RNA (mRNA), and the translation of such mRNA into polypeptide(s). Expression of a gene produces a “gene product.”

As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein, the term “expression vector” refers to a vector comprising a recombinant polynucleotide comprising an expression control sequence operably linked to the nucleotide sequence to be expressed. The expression vector contains sufficient cis-acting elements for expression; other elements for expression can be provided by the host cell or in an in vitro expression system. Expression vectors include expression vectors known in the art, including cosmids, plasmids (for example, naked or contained in liposomes), and viruses incorporating recombinant polynucleotides (for example, lentivirus, retrovirus, adenovirus, and adeno-associated virus).

As used herein, the term “operably linked” or “transcription control” refers to a functional linkage between a regulatory sequence and a heterologous nucleic acid sequence, which results in the expression of the latter. For example, when the first nucleic acid sequence and the second nucleic acid sequence are arranged in a functional relationship, the first nucleic acid sequence and the second nucleic acid sequence are operably linked. For example, if a promoter affects the transcription or expression of a coding sequence, the promoter is operably linked to the coding sequence. The operably linked DNA sequences may be adjacent to each other, and for example, in the case where two protein coding regions need to be linked, the DNA sequences are in the same reading frame.

As used herein, the term “transfer vector” refers to a composition containing an isolated nucleic acid and a substance that can be used to deliver the isolated nucleic acid to the inside of a cell. Many vectors are known in the art, including but not limited to linear polynucleotides, polynucleotides associated with ionic or amphiphilic compounds, plasmids, and viruses. The term transfer vector should also be interpreted to further include non-plasmid and non-viral compounds that facilitate the transfer of nucleic acids into cells, such as polylysine compounds, liposomes, and the like.

As used herein, the term “host cell” can be any type of cell, e.g., a primary cell, a cell in culture, or a cell from a cell line. In specific aspects, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

“Percent (%) amino acid sequence identity” with respect to a polypeptide sequence as set forth herein is defined as the percentage of amino acid residues in a candidate sequence of interest to be compared that are identical with the amino acid residues in a particular polypeptide sequence as set forth herein (e.g. a particular polypeptide sequence characterized by a sequence identifier in the sequence listings), after aligning the sequences and introducing gaps, if necessary, to achieve the maximum percent sequence identity, and not considering any conservative substitutions as part of the sequence identity. A sequence alignment performed for determining percent amino acid sequence identity can be carried out according to procedures known in the art, as described for example in EP 1 241 179 B1, which is incorporated herewith by reference, including in particular page 9, line 35 to page 10, line 40 with the definitions used therein and Table 1 regarding possible conservative substitutions. For example, a skilled person can use publicly available computer software. Computer program methods for determining sequence identity include, but are not limited to BLAST, BLAST-2, ALIGN or Megalign (DNASTAR) software. According to one embodiment, the software alignment program used can be BLAST. A skilled person can determine appropriate parameters for measuring alignment, including any algorithms needed to achieve maximal alignment over the full length of the sequences subjected to comparison. According to one embodiment, the % identity values can be generated using the WU-BLAST-2 computer program (Altschul et al., 1996, Methods in Enzymology 266:460-480, which is incorporated herewith by reference). According to one embodiment, the following parameters are used, when carrying out the WU-BLAST-2 computer program: Most of the WU-BLAST-2 search parameters are set to the default values. The adjustable parameters were set with the following values: overlap span=1, overlap fraction=0.125, word threshold (T)=11, and scoring matrix=BLOSUM62. The HSP S and HSP S2 parameters, which are dynamic values used by BLAST-2, are established by the program itself depending upon the composition of the sequence of interest and composition of the database against which the sequence is being searched. However, the values can be adjusted to increase sensitivity. A % sequence identity value can be determined by dividing (a) the number of matching identical amino acid residues between a particular amino acid sequence as set forth herein which is subjected to comparison (e.g. a particular polypeptide sequence characterized by a sequence identifier in the sequence listings) and the candidate amino acid sequence of interest to be compared, for example the number of matching identical amino acid residues as determined by WU-BLAST-2, by (b) the total number of amino acid residues of the polypeptide sequence as set forth herein which is subjected to comparison (e.g. a particular polypeptide sequence characterized by a SEQ. ID. NO. in the sequence listings).

“Percent (%) nucleic acid sequence identity” with respect to a nucleic acid sequence as set forth herein is defined as the percentage of nucleotides in a candidate sequence of interest to be compared that are identical with the nucleotides in a particular nucleic acid sequence as set forth herein (e.g. a particular polypeptide sequence characterized by a sequence identifier in the sequence listings), after aligning the sequences and introducing gaps, if necessary, to achieve the maximum percent sequence identity.

“As used herein, the term “homology” or “identity” refers to the identity of subunit sequence between two polymer molecules, for example, between two nucleic acid molecules, such as two DNA molecules or two RNA molecules, or between two polypeptide molecules. When subunit positions in two molecules are occupied by the same monomer subunit; for example, if the position of each of two DNA molecules is occupied by adenine, they are homologous or identical at that position. The homology between two sequences is a direct function of the number of matching or homologous positions; for example, if half of the positions in the two sequences (for example, 5 positions in a polymer of 10 subunits in length) are homologous, the two sequences are 50% homologous; if 90% of the positions (for example, 9 out of 10) are matched or homologous, then the two sequences are 90% homologous.

In the context of two or more nucleic acid or polypeptide sequences, identity percent refers to two or more sequences that are the same. When comparing and aligning for maximum correspondence in a comparison window or a designated area, as measured by using one of the following sequence comparison algorithms or by manual alignment and visual inspection, if the two sequences have a specified percentage of identical amino acid residues or nucleotides (e.g., 60% identity, optionally 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity over a specified region, or if not specified, over the entire sequence), then the two sequences are “substantially the same”. Optionally, the identity exists over a region of at least about 50 nucleotides (or 10 amino acids) in length, or more preferably over a region of 100 to 500 or 1000 or more nucleotides in length (Or 20, 50, 200 or more amino acids). For sequence comparison, usually a sequence serves as a reference sequence against which the test sequence is compared. When a sequence comparison algorithm is used, a test sequence and a reference sequence are input into a computer, and the sub-sequence coordinates and the sequence algorithm program parameters are specified, if necessary. Default program parameters can be used, or alternative parameters can be specified. Subsequently, the sequence comparison algorithm calculates the percent sequence identity of the test sequence relative to the reference sequence based on the program parameters. Methods of sequence alignment for comparison are well known in the art as disclosed above.

“Coding sequence” or a sequence “encoding” a particular molecule (e.g., a therapeutic molecule) is a nucleic acid that is transcribed (in the case of DNA) or translated (in the case of mRNA) into polypeptide, in vitro or in vivo, when operably linked to an appropriate regulatory sequence, such as a promoter. The boundaries of the coding sequence are determined by a start codon at the 5′ (amino) terminus and a translation stop codon at the 3′ (carboxy) terminus. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region.

A coding sequence can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the coding sequence.

As used herein, the term “recombinant DNA/RNA technology” refers to the manipulation of nucleic acid sequences outside of an organism. This technology comprises, but is not limited to, combining nucleic acid sequences (e.g., coding sequences, regulatory elements (e.g., promoters, enhancers, silencers, termination sequences), linkers (e.g., spacers, internal ribosome entry sites, cleavage sites) derived from a variety of sources, inserting nucleic acid sequences from a variety of sources in appropriate vectors (e.g., delivery vectors, expression vectors, integrating vectors), modifying or altering nucleotide sequences (e.g., by mutagenesis, insertion of modified nucleotides, 5′-capping, polyadenylation), synthesizing artificial nucleotide sequence. A variety of techniques well-known in the art (e.g., molecular cloning, polymerase chain reaction (PCR), digestion with restriction enzymes, in vitro ligation, mutagenesis, site-directed mutagenesis, prokaryotic and eukaryotic cell transformation or transduction, in vitro DNA/RNA synthesis, in vitro RNA-5′-capping, in vitro RNA-polyadenylation, complementary DNA (cDNA) synthesis, nucleic acid isolation, and the like) can be used to manipulate nucleic acid sequences outside an organism (see for example Green & Sambrook Molecular Cloning: A Laboratory Manual, volumes 1-3, 4th edition).

As used herein, the term “recombinant”, refers to any nucleic acid (e.g., DNA, or RNA), peptide (e.g., oligopeptide, polypeptide, or protein), cell, or organism, which is made by combining genetic material from two or more different sources. In some aspects, the recombinant nucleic acid, peptide, cell or organism comprises a portion of the genetic material from at least one source. In some aspects, “recombinant DNA” molecules can include DNA molecules derived from one organism and inserted in a host organism to produce new genetic combinations. In some aspects, “recombinant RNA” molecule (e.g., recombinant mRNA molecules) can include RNA molecules derived from one organism and inserted in a host organism to produce the expression of a desired genetic product in the host organism. In some aspects, “recombinant peptide” molecules can include amino acid molecules derived from an organism or cell, which are expressed from recombinant nucleic acid molecules.

As used herein, the term “isolated” means changed or removed from the natural state. For example, a nucleic acid or peptide naturally present in a living animal is not “isolated”, but the same nucleic acid or peptide that is partially or completely separated from a substance co-existing in its natural state is “isolated.” The isolated nucleic acid or protein may exist in a substantially purified form or may exist in a non-natural environment such as a host cell.

The term “tumor” as used herein refers to any mass of tissue that results from excessive cell growth or proliferation, either benign (non-cancerous) or malignant (cancerous), including pre-cancerous lesions.

The term “primary tumor”, as used herein, refers to the original, or first, tumor formed in the subject's body.

The term “metastasis”, “metastatic”, “secondary tumor”, or “metastatic tumor”, as used herein, refer to cancer (e.g., a tumor) formed by cancer cells derived from a primary cancer (e.g., tumor) that spread to further locations or areas of the body.

As used herein, the term “specifically binds” refers to an antigen binding molecule that recognizes and binds a protein of a binding partner (such as a tumor antigen) present in a sample, but the antigen binding molecule does not substantially recognize or bind to other molecules in the sample.

As used herein, the term “tumor heterogeneity” means that, after multiple divisions and proliferation during the growth of a tumor, daughter cells of the tumor its show molecular biological or genetic changes, so that there are differences in the growth rate, invasion ability, and drug sensitivity, prognosis and other aspects of the tumor. It is one of the characteristics of malignant tumors.

As used herein, the term “cancer” refers to a broad group of various diseases characterized by the uncontrolled growth of abnormal cells (e.g., malignant cells) in the body. Unregulated cell division and growth results in the formation of malignant tumors that invade neighboring tissues through local spread and can also metastasize to distant parts of the body through the lymphatic system or bloodstream. In some aspects, the methods of the present disclosure can be used to reduce the size of a primary tumor or a metastatic tumor, or treat a primary tumor or a metastatic tumor. The conditions that can be treated or prevented by the method of the present disclosure include, for example, various neoplasms, including benign or malignant tumors, various hyperplasias, and the like. The method of the present disclosure can achieve the inhibition and/or reversal of the undesirable hyperproliferative cell growth involved in such conditions. In some aspects, the cancer can be ovarian cancer.

As used herein, “ovarian cancer” refers to cancers arising in, or involving, the ovaries, e.g. in the epithelium of the ovaries. As used herein, the term “cancer” or “tumor” refers to an uncontrolled growth of cells which interferes with the normal functioning of the bodily organs and systems. A subject that has a cancer or a tumor is a subject having objectively measurable cancer cells present in the subject's body. Included in this definition are benign and malignant cancers, as well as dormant tumors or micrometastases. Cancers which migrate from their original location and seed vital organs can eventually lead to the death of the subject through the functional deterioration of the affected organs. Ovarian cancer is typically treated by cytoreductive surgery (also referred to herein as “debulking”) followed by administration of chemotherapy. As used here, “cytoreductive surgery” refers to surgical removal of at least part of the ovarian cancer tissue from a subject. Cytoreductive surgery can remove varying amounts of tumor tissue from a subject, depending upon the location and character of the tumor tissue, the health of the subject, and complicating factors which one of skill in the art can assess. In some embodiments, cytoreductive surgery can remove at least 10% of the tumor tissue, e.g. 10% or more, 20% or more, 30% or more, 40% or more, 50% or more, 60% or more, 70% or more, 80% or more, 90% or more, or 95% or more of the tumor tissue present in the subject.

As used herein, the term “transfected” or “transformed” or “transduced” refers to the process by which exogenous nucleic acid is transferred or introduced into a host cell. A “transfected” or “transformed” or “transduced” cell is a cell that has been transfected, transformed or transduced with exogenous nucleic acid. The cells include primary cell of a subject and progenies thereof.

As used herein, “refractory” as used herein refers to a disease, such as cancer, which does not respond to treatment. In an embodiment, a refractory cancer may be resistant to treatment before or at the beginning of the treatment. In other embodiments, a refractory cancer may become resistant during treatment. Refractory cancers are also called resistant cancers. In some aspects, refractory or recurrent malignant tumors can use the treatment methods disclosed herein.

As used herein, “relapsed” as used herein refers to the return of the signs and symptoms of a disease (e.g. cancer) or the return of a disease such as cancer during a period of improvement, for example, after a therapy, such as a previous treatment of cancer therapy.

As used herein, the term “combination therapy” means a therapy that includes more than one treatment (e.g., active agent or procedure). In some aspects, the combination therapy herein comprises at least a gene therapy, an anticancer agent, and at least one immune checkpoint inhibitor, which can be administered together or separately. In some aspects, compositions of the combination therapy are formulated together in a single composition or as separate compositions.

As used herein, the terms “treat,” “treated,” and “treating” mean both therapeutic and prophylactic treatment or preventative measures wherein the object is to reverse, alleviate, ameliorate, lessen, inhibit, slow down progression, development, severity or recurrence of an undesired symptom, complication, condition, biochemical indicia of a disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. In some aspects, treatment includes eliciting a clinically significant response without excessive levels of side effects. In some aspects, treatment includes prolonging survival as compared to expected survival if not receiving treatment. As used herein, the term “amelioration” or “ameliorating” refers to a lessening of severity of at least one indicator of a condition or disease. As used herein, the term “preventing” or “prevention” refers to delaying or forestalling the onset, development or progression of a condition or disease for a period of time, including weeks, months, or years. As used herein, the term “prophylactic” (e.g., “prophylactic agent”, “prophylactic treatment”, “prophylactically effective amount”), refers to any complete or partial prevention of a disease or symptom thereof and/or can be therapeutic in terms of a partial or complete cure for a disease and/or adverse effect and/or symptom attributable to the disease.

As used herein, the terms “individual” and “subject” have the same meaning herein, and can be a human and animal from other species. As used herein, the terms “subject” and “patient” are used interchangeably. The subject can be an animal. In some aspects, the subject is a mammal such as a non-human animal (e.g., cow, pig, horse, cat, dog, rat, mouse, monkey or other primate, etc.). In some aspects, the subject is a human. In some aspects, the patient is a subject who has a disease, disorder, or condition, or is at risk of suffering from a disease, disorder, or condition, or is otherwise in need of the compositions and methods provided herein.

As used herein, the terms “therapeutically effective amount”, “therapeutically effective”, “effective amount” or “in an effective amount” are used interchangeably herein and refer to the amount of a compound, preparation, substance or composition that is effective to achieve a specific biological result as described herein, such as but not limited to treating or reducing the growth of a cancer or tumor. When indicating “immunologically effective amount”, “anti-tumor effective amount”, “tumor-suppressing effective amount” or “therapeutically effective amount”, the precise number of immune effector cells and therapeutic agents of the present disclosure to be administered can be determined by a physician in consideration of the individual's age, weight, tumor size, degree of infection or metastasis, and the condition of a patient (subject). An effective amount of immune effector cells refers to, but is not limited to, the number of immune effector cells which can increase, enhance or prolong the anti-tumor activity of immune effector cells; increase the number of anti-tumor immune effector cells or activated immune effector cells; promote tumor regression, tumor shrinkage and/or tumor necrosis.

The term “pharmaceutically acceptable” as used herein refers to those compounds, materials, compositions, formulations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “excipient” refers to any substance, not itself a therapeutic agent, which can be used in a composition for delivery of an active therapeutic agent to a subject or combined with an active therapeutic agent (e.g., to create a pharmaceutical composition) to improve its handling or storage properties or to permit or facilitate formation of a dose unit of the composition. Excipients include, but are not limited to, solvents, penetration enhancers, wetting agents, antioxidants, lubricants, emollients, substances added to improve appearance or texture of the composition and substances used to form hydrogels. Any such excipients can be used in any dosage forms according to the present disclosure. The foregoing classes of excipients are not meant to be exhaustive but merely illustrative as a person of ordinary skill in the art would recognize that additional types and combinations of excipients could be used to achieve the desired goals for delivery of a drug. The excipient can be an inert substance, an inactive substance, and/or a not medicinally active substance. The excipient can serve various purposes.

A person skilled in the art can select one or more excipients with respect to the particular desired properties by routine experimentation and without any undue burden. The amount of each excipient used can vary within ranges conventional in the art. Techniques and excipients which can be used to formulate dosage forms are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21st edition, Gennaro, Ed., Lippincott Williams & Wilkins (2005).

As used herein, the term “immune response” refers to a biological response within an organism against a foreign agent or abnormal cell (e.g., a tumor cell), wherein the response protects the organism against such agents/cells and diseases caused by them. An immune response is mediated by the action of a cell of the immune system (e.g., a T lymphocyte (T cell), B lymphocyte (B cell), natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the organism's body of invading pathogens, cells or tissues infected with pathogens, cancerous or other abnormal cells, or, in cases of autoimmunity or pathological inflammation, normal human cells or tissues. In some aspects, an immune reaction includes, e.g., activation or inhibition of a T cell, e.g., an effector T cell or a Th cell, such as a CD4+ or CD8+ T cell, or the inhibition of a regulatory T cell (Treg cell).

As used herein, the term “autologous” refers to any material derived from an individual that will later be reintroduced into that same individual.

The term “antibody” herein is used in the broadest sense and encompasses various antibody structures, including but not limited to monoclonal antibodies, multispecific antibodies (e.g., bispecific antibodies), and antibody fragments so long as they exhibit the desired antigen-binding activity.

Papain digestion of intact antibodies produces two identical antigen-binding fragments, called “Fab” fragments containing each the heavy- and light-chain variable domains (VH and VL, respectively) and also the constant domain of the light chain (CL) and the first constant domain of the heavy chain (CH1). The term “Fab fragment” thus refers to an antibody fragment comprising a light chain comprising a VL domain and a CL domain, and a heavy chain fragment comprising a VH domain and a CH1 domain.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′) 2; diabodies; linear antibodies; single-chain antibody molecules (e.g. scFv); and multispecific antibodies formed from antibody fragments.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs), including the complementarity determining regions (CDRs) (see, e.g., Kindt et al. Kuby Immunology, 6th ed., W.H. Freeman and Co., page 91 (2007)).

A “paratope” or an “antigen binding site”, as used interchangeably herein, refers to a part of an antibody which recognizes and binds to an antigen. An antigen binding site is formed by several individual amino acid residues from the antibody's heavy and light chain variable domains arranged that are arranged in spatial proximity in the tertiary structure of the Fv region. In one embodiment, the antigen binding site is defined as a set of the six CDRs comprised in a cognate VH/VL pair.

The term “complementarity determining regions” or “CDRs” as used herein refers to each of the regions of an antibody variable domain which are hypervariable in sequence and contain antigen-contacting residues. Generally, antibodies comprise six CDRs: three in the VH domain (CDR-H1, CDR-H2, CDR-H3), and three in the VL domain (CDR-L1, CDR-L2, CDR-L3). Unless otherwise indicated, CDR residues and other residues in the variable domain (e.g., FR residues) are numbered herein according to the Kabat numbering system (Kabat et al., Sequences of Proteins of Immunological Interest, 5th Ed. Public Health Service, National Institutes of Health, Bethesda, Md., 1991).

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below.

“Framework” or “FR” as used herein refers to variable domain amino acid residues other than CDR residues. The framework of a variable domain generally consists of four framework domains: FR1, FR2, FR3, and FR4. Accordingly, the CDR and FR amino acid sequences generally appear in the following sequence in the (a) VH domain: FR1-CDR-H1-FR2-CDR-H2-FR3-CDR-H3-FR4; and (b) in the VL domain: FR1-CDR-L1-FR2-CDR-L2-FR3-CDR-L3-FR4.

As used herein, “affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art, including those described herein. Specific illustrative and exemplary embodiments for measuring binding affinity are described herein.

As used herein, the term “epitope” denotes the site on an antigen, either proteinaceous or non-proteinaceous, to which an antibody binds. Epitopes can be formed both from contiguous amino acid stretches (linear epitope) or comprise non-contiguous amino acids (conformational epitope), e.g. coming in spatial proximity due to the folding of the antigen, i.e. by the tertiary folding of a proteinaceous antigen. Linear epitopes are typically still bound by an antibody after exposure of the proteinaceous antigen to denaturing agents, whereas conformational epitopes are typically destroyed upon treatment with denaturing agents. An epitope comprises at least 3, at least 4, at least 5, at least 6, at least 7, or 8-10 amino acids in a unique spatial conformation.

Screening for antibodies binding to a particular epitope (i.e., those binding to the same epitope) can be done using methods routine in the art such as, e.g., without limitation, alanine scanning, peptide blots (see Meth. Mol. Biol. 248 (2004) 443-463), peptide cleavage analysis, epitope excision, epitope extraction, chemical modification of antigens (see Prot. Sci. 9 (2000) 487-496), and cross-blocking (see “Antibodies”, Harlow and Lane (Cold Spring Harbor Press, Cold Spring Harb., NY).

Antigen Structure-based Antibody Profiling (ASAP), also known as Modification-Assisted Profiling (MAP), allows to bin a multitude of monoclonal antibodies specifically binding to an antigen based on the binding profile of each of the antibodies from the multitude to chemically or enzymatically modified antigen surfaces (see, e.g., US 2004/0101920). The antibodies in each bin bind to the same epitope which may be a unique epitope either distinctly different from or partially overlapping with epitope represented by another bin. Also competitive binding can be used to easily determine whether an antibody binds to the same epitope, or competes for binding with, a reference antibody. For example, an “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. Also for example, to determine if an antibody binds to the same epitope as a reference antibody, the reference antibody is allowed to bind to its antigen under saturating conditions. After removal of the excess of the reference antibody, the ability of an antibody in question to bind to its antigen is assessed. If the antibody is able to bind to its antigen after saturation binding of the reference antibody, it can be concluded that the antibody in question binds to a different epitope than the reference antibody. But, if the antibody in question is not able to bind to its antigen after saturation binding of the reference antibody, then the antibody in question may bind to the same epitope as the epitope bound by the reference antibody. To confirm whether the antibody in question binds to the same epitope or is just hampered from binding by steric reasons routine experimentation can be used (e.g., peptide mutation and binding analyses using ELISA, RIA, surface plasmon resonance, flow cytometry or any other quantitative or qualitative antibody-binding assay available in the art). This assay should be carried out in two set-ups, i.e. with both of the antibodies being the saturating antibody. If, in both set-ups, only the first (saturating) antibody is capable of binding to the antigen of interest, then it can be concluded that the antibody in question and the reference antibody compete for binding to the same antigen.

Sometimes two antibodies are deemed to bind to the same epitope if essentially all amino acid mutations in the antigen that reduce or eliminate binding of one antibody also reduce or eliminate binding of the other. Two antibodies are deemed to have “overlapping epitopes” if only a subset of the amino acid mutations that reduce or eliminate binding of one antibody reduce or eliminate binding of the other.

As used herein, the term “anti-tumor effect” refers to a biological effect that can be manifested in various ways, including but not limited to, for example, reduction in tumor volume, reduction in the number of tumor cells, reduction in the number of metastases, increase in life expectancy, reduction in tumor cell proliferation, and reduction in tumor cell survival rate, or improvement in various physiological symptoms related to cancerous conditions. The “anti-tumor effect” can also be expressed by the ability of the peptides, polynucleotides, cells and antibodies of the present disclosure to prevent or reduce the frequency of tumorigenesis.

The term “chemotherapy” or “chemotherapeutic agent” as used herein refers to a wide variety of chemotherapeutic agents that may be used in accordance with the present embodiments. The term “chemotherapy” refers to the use of drugs to treat cancer. A “chemotherapeutic agent” is used to connote a compound or composition that is administered in the treatment of cancer.

The term “pharmaceutical composition” refers to a preparation which is in such form as to permit the biological activity of the active ingredient to be effective, and which contains no additional components which are unacceptably toxic to a subject to which the composition would be administered. The composition can be sterile.

One skilled in the art will readily recognize that vectors, polynucleotides, and pharmaceutical compositions of the present disclosure, or combinations thereof, can be readily incorporated into one of the established kit formats which are well known in the art.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature.

### II. GEN-1 (Nanoparticle Formulated IL-12 DNA)

In animal models, recombinant IL-12 has been demonstrated to induce profound T-cell mediated antitumor effects causing regression of established tumors, followed by systemic immune memory. See The Oncologist, 1996, vol. 1, 88. However, systemic administration of recombinant IL-12 has resulted in dose limiting toxicity in several experimental trials and in an initial human trial. See Lab Invest., 1994, vol. 71, 862; Science, 1995, vol. 270, 908; J. Interferon Cytokine Res., 1995, vol. 14, 335. Dose limiting toxicity was also observed with intraperitoneal administration of recombinant IL-12 in a recent human clinical trial. Clin. Cancer Res., 2002, vol. 8, 3686. A gene delivery approach that can provide therapeutic levels of IL-12 locally at the tumor site would have the advantage of generating an anticancer response without causing systemic toxicity.

Both viral and non-viral gene delivery systems have been used for IL-12 gene delivery in animal models of cancer. The viral approach has serious practical limitations due to toxicity concerns mainly because of an increased incidence of cancer and a strong immune reaction to viral antigens by the host system. There is considerable interest in the development of non-viral gene delivery systems due to their lesser toxicity. Using polyvinylpyrrolidone (PVP), a non-viral gene delivery system, for the delivery of IL-12 to treat renal carcinoma (Renca) and colon cell carcinoma (CT26) has been demonstrated. See Gene Ther., 1999, vol. 6, 833. When tumors were subjected to this gene therapy, they displayed all the characteristics of IL-12 protein therapy, e.g., an increased infiltration of NK cells, CD4 and CD8 T cells, coupled with an increased expression of major histocompatibility complex (MHC) class I molecules. IL-12 gene delivery was well tolerated and highly effective against both Renca and CT26 tumor bearing animals. Tumor rejecting mice were also protected from a subsequent rechallenge, suggesting the presence of a long lasting systemic immunity. A functionalized and less toxic water soluble lipopolymer (WSLP) has been tested for delivery of the IL-12 gene to CT26 colon carcinoma tumors. See Mahato et al, Mol. Ther., 2001, vol. 4, 130. IL-12 plasmid (pIL-12) and WSLP (pIL-12/WSLP) treatment gave higher levels of intratumoral gene expression than naked DNA.

Interleukin-12 (IL-12) is a pro-inflammatory cytokine that plays an important role in innate and adaptive immunity. Gately, M K et al., Annu Rev Immunol. 16:495-521 (1998). IL-12 functions primarily as a 70 kDa heterodimeric protein consisting of two disulfide-linked p35 and p40 subunits. IL-12 p40 homodimers do exist, but other than functioning as an antagonist that binds the IL-12 receptor, they do not appear to mediate a biologic response. Id. The precursor form of the IL-12 p40 subunit (e.g., NM_002187; NP_002178; also referred to as IL-12B, natural killer cell stimulatory factor 2, cytotoxic lymphocyte maturation factor 2) is 328 amino acids in length, while its mature form is 306 amino acids long. The precursor form of the IL-12 p35 subunit (e.g., NM_000882; NP_000873; also referred to as IL-12A, natural killer cell stimulatory factor 1, cytotoxic lymphocyte maturation factor 1) is 219 amino acids in length and the mature form is 197 amino acids long. Id. The genes for the IL-12 p35 and p40 subunits reside on different chromosomes and are regulated independently of each other. Gately, M K et al., Annu Rev Immunol. 16:495-521 (1998). Many different immune cells (e.g., dendritic cells, macrophages, monocytes, neutrophils, and B cells) produce IL-12 upon antigenic stimuli. The active IL-12 heterodimer is formed following protein synthesis. Id.

Due to its ability to activate both NK cells and cytotoxic T cells, IL-12 protein has been studied as a promising anti-cancer therapeutic since 1994. See Nastala, C. L. et al., J Immunol 153:1697-1706 (1994). But despite high expectations, early clinical studies did not yield satisfactory results. Lasek W. et al., Cancer Immunol Immunother 63:419-435, 424 (2014). Repeated administration of IL-12, in most patients, led to adaptive response and a progressive decline of IL-12-induced interferon gamma (IFNy) levels in blood. Id. Moreover, while it was recognized that IL-12-induced anti-cancer activity is largely mediated by the secondary secretion of IFNy, the concomitant induction of IFNy along with other cytokines (e.g., TNF-α) or chemokines (IP-10 or MIG) by IL-12 caused severe toxicity. Id.

In addition to the negative feedback and toxicity, the marginal efficacy of the IL-12 therapy in clinical settings may be caused by the strong immunosuppressive environment in humans.

Furthermore, secondary effects of the cytokine IL-12 production, namely IFN-γ and nitric oxide (NO) levels were also higher in WSLP treated tumors when compared with naked DNA. A single injection of pIL-12/WSLP complexes produced suboptimal effects on tumor growth and animal survival, while repeated delivery yielded better efficacy which indicates insufficient delivery by the system. J. Control Release 2003, vol. 87, 177. Similarly, intratumoral injection of IL-12 plasmid in another polymeric carrier, PAGA, produced only partial inhibition of CT26 tumors. See Gene Ther., 2002, vol. 9, 1075. These results warrant the need for more efficient delivery systems. Despite their insufficiencies in earlier preclinical trials, the excellent molecular flexibility of polymeric gene carriers allows for complex modification and novel functionalization imperative for the development of more efficient gene delivery systems.

To achieve a desirable outcome from a combination approach involving gene therapeutics, the selection of an appropriate gene delivery system is important. The gene delivery system used in the aforementioned combination experiments (Molecular Therapy, 2004, vol. 9, 829) is a water soluble lipopolymer, PEI-Cholesterol (WSLP).

IL-12 is mainly secreted from antigen presenting cells (phagocytes, dendritic cells) in response to pathogens, promoting CD4+ cell differentiation into Th1 cells. It has a positive synergistic proliferative effect on pre-activated NK and T cells, enhances independently and/or synergistically the cytolytic ability of both NK and CD8+ T cells by upregulation of genes that encode cytotoxic cell granule-associated proteins. It also increases ADCC in antibody coated tumors at considerably lower concentrations than IL-2. Effector cells stimulated by IL-12 produce several cytokines such as GMCSF and TNF-α but primarily IFN-γ. IL-12 consists of a p35 and a p40 subunit, the latter also shared by IL-23. The IL-12 receptor (IL-12R) consists of two chains, IL-12Rβ1 and IL-12Rβ213 and signals predominately through STAT4. The IL-12R is expressed mainly by activated NK and T cells; it is barely detectable in resting T cells but is expressed at a low level in NK, probably explaining their rapid response to IL-12. Nevertheless, TCR activation and co-stimuli like B7, IFN-α, IFN-γ and IL-12 itself upregulate IL-12R expression (particularly IL-12RB2).

Antitumor activity of systemic or local administration of IL-12 has been established in preclinical studies against various tumor cell lines. Dose and model dependent response as well as a memory antitumor effect were observed. Other studies suggest that IL-12 is more efficacious against early stage or microscopic tumors than advanced disease. The T cell response is responsible for its antitumor activity and NK/NKT cell activation seems to prevent metastasis. In addition to effector T cell (Teff) mediated tumor control, IL-12 inhibits tumor derived regulatory T cells (Treg) either through suppression of T cell IL-2 production, induction of apoptosis or by IFN-γ mediated cell arrest, thus enhancing the Teff/Treg ratio both in vitro and in vivo. IL-12 was also reported to mediate reprogramming of intratumoral myeloid derived suppressor cells (MDSC) to enhance CTL activity in a B16 melanoma model, reversing their suppressive role in vivo. IL-12RB2 has also emerged as a potential tumor suppressor gene, particularly in hematologic malignancies where epigenetic IL-12RB2 gene silencing by promoter hypermethylation or gene downregulation were observed in multiple B-cell myeloma specimens and IL-12 significantly reduced tumor load upon receptor restoration. Consistently, IL-12RB2−/−mice develop spontaneous malignancies and lymphoproliferative diseases. IL-12 also has “direct” antitumor activity in IL-12RB1 expressing tumors through IFN-γ mediated upregulation of MHC Class I molecules.

Initial clinical trials were temporarily halted after unexpected toxicity related deaths were observed in phase II trial in patients with metastatic RCC and melanoma, although the precedent phase I trial had established a safe MTD. This was attributed to a slight, though biologically significant alteration in the treatment schedule involving elimination of a test dose prior to a 5 day treatment course. Evaluation suggested that the test dose attenuated the effect of subsequent IL-12 on IFN-γ production through as yet unknown mechanism. Various IL-12 regimens were subsequently tested but with modest clinical results in patients with solid tumors and more promising results in hematologic malignancies. Since IL-12 is a key mediator of the Th1 response, efforts focused on using IL-12 as an adjunct to vaccine therapy against tumor associated antigens, both for resectable or metastatic disease. In general, IL-12 has improved immune responses but this did not translate into clinically significant anti-tumor effects.

The demonstration in preclinical models that locally delivered IL-12 was efficacious without the detriments of systemic administration prompted research into development of ways to express IL-12 intratumorally (IT). A study of IT injection of IL-12 into the primary tumor of patients with head and neck cancer revealed induced B cell activation that correlated with increased survival. Rendering tumor cells able to produce IL-12 has been of interest and a number of small studies using viral vectors or plasmid DNA tested this approach with limited results. Antibody formation against viral vectors and undocumented efficiency of gene delivery were limiting factors. A new way to administer IL-12 locally is by the injection of plasmid DNA coding for IL-12 and electroporation of superficial tumors to facilitate cell entry. Results of a phase I study demonstrated antitumor activity, both at local injection sites and systemic uninjected sites. Two trials in patients with gynecological malignancies assessed a PEG-PEI-cholesterol lipopolymer formulated IL-12 plasmid delivered by intraperitoneal administration but clinical benefit was modest. Current studies are also testing IL-12 as gene therapy including gene electroporation mediated plasmid transfer, adenoviral vector combined with orally administered activator ligand or mesenchymal engineered cells for IL-12 expression. NHS-IL-12 is as immunokine consisting of two IL-12 molecules fused to a tumor necrosis-targeting human IgG1 with longer half-life. It has now entered phase II clinical trial testing.

Carson et al., conducted an NCI-sponsored phase I trial to determine the safety and optimal biologic dose of IL-12 when given in combination with trastuzumab. Patients with metastatic HER2-positive malignancies received trastuzumab on day 1 of each weekly cycle (4 mg/kg initially and 2 mg/kg thereafter). Beginning in week 3, patients also received intravenous (i.v.) injections of IL-12 on days 2 and 5 of the weekly cycle. The IL-12 component was dose-escalated within cohorts of three patients (30, 100, 300, or 500 ng/kg). Fifteen patients were treated. The regimen was well-tolerated and no drug-related Grade 3 or 4 toxicities were noted. Evaluation of dose-limiting toxicity and biologic endpoints suggested that the 300 ng/kg dose was both the maximally tolerated dose and the optimal biologic dose of IL-12 for use in combination with trastuzumab. Correlative assays showed sustained production of IFN-γ by NK cells only in the three patients that exhibited a favorable response to the regimen. Enhanced circulating levels of NK cell-derived chemokines (MIP-1a, IL-8, and RANTES) were also detected in these three patients. These factors exerted strong chemotactic effects on naïve and activated T cells and their presence correlated with the infiltration of tumor tissue by CD8+ T cells. Importantly, NK cell-mediated ADCC against tumor targets in vitro did not appear to correlate with clinical response or dose of IL-12. Thus, as predicted, the clinical response to IL-12/trastuzumab therapy was associated with the production of IFN-γ and chemokines by immune cells.

Carson et al., conducted a follow-up trial that employed IL-12 in combination with trastuzumab and paclitaxel. Paclitaxel was given intravenously (i.v.) at 175 mg/m² every 3 weeks. Trastuzumab was given on day 1 each week (4 mg/kg initially and 2 mg/kg thereafter) in combination with injections of IL-12 on days 2 and 5 starting in cycle 2. This trial accrued 21 patients with metastatic 2+ and 3+HER2-positive tumors, 16 of whom had received prior chemotherapy. The IL-12 component was dose-escalated in cohorts of three patients (100 ng/kg and then 300 ng/kg), but because of dose-limiting grade 3 fatigue at the 300 ng/kg dose level, the IL-12 component was reduced to 200 ng/kg subcutaneously. Overall there was a 52% rate of clinical benefit. There was significantly increased activation of ERK in peripheral blood mononuclear cells and increased plasma levels of IFN-γ with clinical benefit (complete response, partial response, or stable disease), but not in patients with progressive disease. No patient with progressive disease had measurable levels of IFN-γ in their circulation. Within any one cycle, IFN-γ levels typically peaked following injections of IL-12 (range 124-1612 μg/ml) and then fell to baseline. Analysis of patient time-to-progression data using the nonparametric Mann-Whitney U test indicated that induction of IFN-γ was associated with a statistically significant increase in progression-free survival (p=0.004). Intracellular flow cytometric analysis of cryopreserved PBMCs from day 5 of the treatment cycle revealed high levels of IFN-γ within CD56+NK cells only in those patients who exhibited a clinical response or stabilization of disease. Circulating levels of MIP-1a also correlated with clinical response, as did levels of IP-10 and MIG, two anti-angiogenic factors induced by IFN-γ.

A GOG trial evaluated GEN-1 in 16 patients with persistent or recurrent platinum-resistant EOC. In this study higher doses of GEN-1 were assessed in combination with intravenous pegylated liposomal doxorubicin (PLD) 40 mg/m² (dose level 1 and 2) or 50 mg/m² (dose level 3) every 28 days and intraperitoneal GEN-1 at 24 mg/m² (dose level 1) or 36 mg/m² (dose level 2 and 3) on days 1, 8, 15, and 22 of a 28-day cycle. Cycles were repeated every 28 days until disease progression. Clinical benefit of 57.1% (PR=21.4%; SD-35.7%) was found in the 14 patients with measurable disease. The highest number of partial responses (28.6%) and stable disease (57.1%) were found at dose level 3. The maximum tolerated dose was not reached. Increases in IL-12, IFN-γ, and TNF-α levels were found in peritoneal fluid following GEN-1 treatment.

Ovarian cancer represents the fifth most common form of cancer affecting women and is the most lethal of gynecological malignancies, ranking fourth in cancer deaths among women. First-line chemotherapy regimens for the treatment of ovarian cancer are typically platinum-based combination therapies that are administered intravenously (IV) for 4 to 6 treatments every 21 to 28 days. Although nearly 90% of women respond initially to platinum-based therapies, 55-75% of women will develop recurrent ovarian cancer within 2 years. The second and third line therapies are generally ineffective and toxic including immune checkpoint inhibitors with a 11-15% response rate in platinum resistant, recurrent patients. There is an immediate need for safer and more effective therapies for the treatment of ovarian cancer.

Cancers can evade detection and destruction by the immune system despite the fact that many tumors elicit a strong immune response evident in lymphocyte infiltrates of the primary lesion. Tumor immune evasion can be categorized into induction of immune tolerance and resistance to killing by activated immune effector cells. The ‘immunoediting’ hypothesis suggests that tumors manipulate their microenvironment by creating complex local and regional immunosuppressive networks comprising various tumor-derived cytokines and other soluble factors. Therefore, by the time tumors have become clinically detectable, the tumor has already evolved mechanisms to evade the immune response mounted by the host against it. Such resistance mechanisms must be overcome to create effective and durable antitumor immunity. One of the most promising strategies to enhance the patient's antitumor response appears to be the use of antibodies that block the immunoregulatory mechanisms that can suppress host responses to tumor associated antigens with significant results demonstrated to date with CTLA4 and PD1/PDL1 blocking monoclonal antibodies. However, a sizable majority of patients with solid tumors do not respond to these agents. In these cases, the tumor microenvironment (TME) is considered less immunogenic and proinflammatory cytokines such as IL-12 may play an important role in transforming the TME working synergistically with CTLA4 blockade and PD1 blockade.

The GEN-1 nanoparticle comprises a DNA plasmid encoding IL-12 gene and a synthetic polymer facilitating plasmid delivery. GEN-1 is designed to be delivered locally (e.g., intraperitoneally), offering the potential for cytokines to be expressed specifically in the tumor micro-environment with the goal of achieving increased efficacy while minimizing potential systemic toxicity. GEN-1 has been studied in subjects with recurrent ovarian cancer as a single agent or combination agent with standard chemotherapy. A phase I monotherapy trial evaluating 13 platinum-resistant EOC patients who were treated with IP GEN-1 at 0.6 mg/m², 3 mg/m², 12 mg/m² and 24 mg/m² weekly every 4 weeks evaluating for safety and tolerability. The most frequent adverse events (AE) were fever and abdominal pain. Significant increases in IFN-γ were observed in peritoneal fluid but not in the serum. Another phase I trial was conducted in platinum-sensitive EOC patients treated with intravenous (IV) carboplatin and docetaxel with escalating doses of IP GEN-1:12 mg/m², 18 mg/m², or 24 mg/m²×4 doses or 24 mg/m²×6-8 doses. Similar AEs and dose dependent rises in IFN-γ concentrations were seen. No dose-limiting toxicities (DLT) were seen and the MTD was not reached. The addition of GEN-1 to chemotherapy did not attenuate the efficacy or exacerbate the side effects.

A GOG trial evaluated GEN-1 in 16 patients with persistent or recurrent platinum-resistant EOC. In this study higher doses of GEN-1 were assessed in combination with intravenous pegylated liposomal doxorubicin (PLD) 40 mg/m² (dose level 1 and 2) or 50 mg/m² (dose level 3) every 28 days and intraperitoneal GEN-1 at 24 mg/m² (dose level 1) or 36 mg/m² (dose level 2 and 3) on days 1, 8, 15, and 22 of a 28-day cycle. Cycles were repeated every 28 days until disease progression. Clinical benefit of 57.1% (PR=21.4%; SD=35.7%) was found in the 14 patients with measurable disease. The highest number of partial responses (28.6%) and stable disease (57.1%) were found at dose level 3. The maximum tolerated dose was not reached. Increases in IL-12, IFN-γ, and TNF-α levels were found in peritoneal fluid following GEN-1 treatment.

Translational research findings demonstrate that GEN-1 in ovarian cancer subjects is biologically active and promotes a pro-immune T-cell population dynamic and conversion of tumor naïve T-cells into cytotoxic effector T-cells in the tumor microenvironment. To date over 100 patients with ovarian cancer have been dosed with GEN-1. Most common adverse event attributed to GEN-1 at higher doses are: abdominal pain, nausea, fatigue, pyrexia, vomiting and diarrhea.

In some aspects, the GEN-1 DNA plasmid is delivered using a synthetic polymer facilitating plasmid delivery that is a lipopolymer.

In some aspects, the lipopolymer comprises a polyethyleneimine (PEI) covalently linked independently to cholesterol and polyethylene glycol (PEG) groups.

The present disclosure provides a polymeric system, PEG-PEI-Cholesterol (PPC), which differs from WSLP (PEI-Cholesterol) in that it contains PEG moieties and yields significantly higher transfection efficiency in tumors. The addition of PEG is designed to enhance the stability of the nucleic acid/polymer complexes in the biological milieu to circumvent for this deficiency in the prior art (WSLP). Furthermore, the addition of PEG chains allows for the incorporation of ligands on to the PPC chain to improve the tissue selectivity of delivery. For example, the cholesterol moiety which is directly linked to the PEI back bone in the prior art (WSLP) may be extended farther from the PEI backbone to create a more flexible geometry for cell receptor interaction. Controlling the number of PEG molecules per unit of the PEI backbone is important to achieve optimal enhancement in transfection activity. A preferred range of composition was a PEG:PEI molar ratio of 2-4 at a fixed cholesterol content. The optimal ratio between PEI and cholesterol was 1:0.5 to 1:1.

Certain aspects of the disclosure are related to a combination therapy comprising: a (i) a nucleic acid vector (e.g., a plasmid) comprising a polynucleotide that encodes an interleukin-12 (IL-12) formulated with a lipopolymer (e.g., a nanoparticle); and (ii) an immune checkpoint inhibitor.

Certain aspects of the disclosure are related to a method of treating a subject suffering from cancer comprising administering to the subject a combination therapy comprising: (i) a nucleic acid vector (e.g., a plasmid) comprising a polynucleotide that encodes an interleukin-12 (IL-12) formulated with a lipopolymer (e.g., a nanoparticle); and (ii) an immune checkpoint inhibitor.

In some aspects, the polynucleotide encodes human IL-12. In some aspects, the polynucleotide encodes a p35 subunit of IL-12 and a p40 subunit of IL-12.

In some aspects, nucleic acid vector (e.g., a plasmid) comprises a promoter operably linked to a nucleic acid encoding a p35 subunit of IL-12 and a promoter operably linked to a nucleic acid encoding a p40 subunit of IL12.

In some aspects, the human IL-12 p35 comprises an amino acid sequence having at least 70%, at least 75%, at least 80%, at least 85%, at least 90%, at least 95%, or 100% identity to SEQ ID NO: 85. In some aspects, the human IL-12 p40 comprises an amino acid sequence having at least 70%, at least 75%, at least 80%, at least 85%, at least 90%, at least 95%, or 100% identity to SEQ ID NO: 86.

In some aspects, the human IL-12 p35 comprises the sequence:

(SEQ ID NO: 85)
MGPARSLLLVATLVLLDHLSLARNLPVATPDPGMFPCLHHSQNLLRAVS
NMLQKARQTLEFYPCTSEEIDHEDITKDKTSTVEACLPLELTKNESCLN
SRETSFITNGSCLASRKTSFMMALCLSSIYEDLKMYQVEFKTMNAKLLM
DPKRQIFLDQNMLAVIDELMQALNFNSETVPQKSSLEEPDFYKTKIKLC
ILLHAFRIRAVTIDRVMSYLNAS.

In some aspects, the human IL-12 p40 comprises the sequence:

(SEQ ID NO: 86)
MGHQQLVISWFSLVFLASPLVAIWELKKDVYVVELDWYPDAPGEMVVLT
CDTPEEDGITWTLDQSSEVLGSGKTLTIQVKEFGDAGQYTCHKGGEVLS
HSLLLLHKKEDGIWSTDILKDQKEPKNKTFLRCEAKNYSGRFTCWWLTT
ISTDLTFSVKSSRGSSDPQGVTCGAATLSAERVRGDNKEYEYSVECQED
SACPAAEESLPIEVMVDAVHKLKYENYTSSFFIRDIIKPDPPKNLQLKP
LKNSRQVEVSWEYPDTWSTPHSYFSLTFCVQVQGKSKREKKDRVFTDKT
SATVICRKNASISVRAQDRYYSSSWSEWASVPCS.

In some aspects, the polynucleotide encoding the human IL-12 p35 has at least 70%, at least 75%, at least 80%, at least 85%, at least 90%, at least 95%, or 100% identity to SEQ ID NO: 87. In some aspects, the polynucleotide encoding the human IL-12 p35 has at least 70%, at least 75%, at least 80%, at least 85%, at least 90%, at least 95%, or 100% identity to SEQ ID NO: 88.

In some aspects, the polynucleotide encoding the human IL-12 p35 comprises the sequence:

(SEQ ID NO: 87)
atgggtccagcgcgcagcctcctccttgtggctaccctggtcctcctgg
accacctcagtttggccagaaacctccccgtggccactccagacccagg
aatgttcccatgccttcaccactcccaaaacctgctgagggccgtcagc
aacatgctccagaaggccagacaaactctagaattttacccttgcactt
ctgaagagattgatcatgaagatatcacaaaagataaaaccagcacagt
ggaggcctgtttaccattggaattaaccaagaatgagagttgcctaaat
tccagagagacctctttcataactaatgggagttgcctggcctccagaa
agacctcttttatgatggccctgtgccttagtagtatttatgaagactt
gaagatgtaccaggtggagttcaagaccatgaatgcaaagcttctgatg
gatcctaagaggcagatctttctagatcaaaacatgctggcagttattg
atgagctgatgcaggccctgaatttcaacagtgagactgtgccacaaaa
atcctcccttgaagaaccggatttttataaaactaaaatcaagctctgc
atacttcttcatgctttcagaattcgggcagtgactattgatagagtga
tgagctatctgaatgcttcctaa.

In some aspects, the polynucleotide encoding the human IL-12 p35 comprises the sequence:

(SEQ ID NO: 88)
Atgggtcaccagcagttggtcatctcttggttttccctggtttttctgg
catctcccctcgtggccatatgggaactgaagaaagatgtttatgtcgt
agaattggattggtatccggatgcccctggagaaatggtggtcctcacc
tgtgacacccctgaagaagatggtatcacctggaccttggaccagagca
gtgaggtcttaggctctggcaaaaccctgaccatccaagtcaaagagtt
tggagatgctggccagtacacctgtcacaaaggaggcgaggttctaagc
cattcgctcctgctgcttcacaaaaaggaagatggaatttggtccactg
atattttaaaggaccagaaagaacccaaaaataagacctttctaagatg
cgaggccaagaattattctggacgtttcacctgctggtggctgacgaca
atcagtactgatttgacattcagtgtcaaaagcagcagaggctcttctg
acccccaaggggtgacgtgcggagctgctacactctctgcagagagagt
cagaggggacaacaaggagtatgagtactcagtggagtgccaggaggac
agtgcctgcccagctgctgaggagagtctgcccattgaggtcatggtgg
atgccgttcacaagctcaagtatgaaaactacaccagcagcttcttcat
cagggacatcatcaaacctgacccacccaagaacttgcagctgaagcca
ttaaagaattctcggcaggtggaggtcagctgggagtaccctgacacct
ggagtactccacattcctacttctccctgacattctgcgttcaggtcca
gggcaagagcaagagagaaaagaaagatagagtcttcacggacaagacc
tcagccacggtcatctgccgcaaaaatgccagcattagcgtgcgggccc
aggaccgctactatagctcatcttggagcgaatgggcatctgtgccctg
cagttagac.

In some aspects, nucleic acid vector (e.g., a plasmid) comprises an intron, a 3′

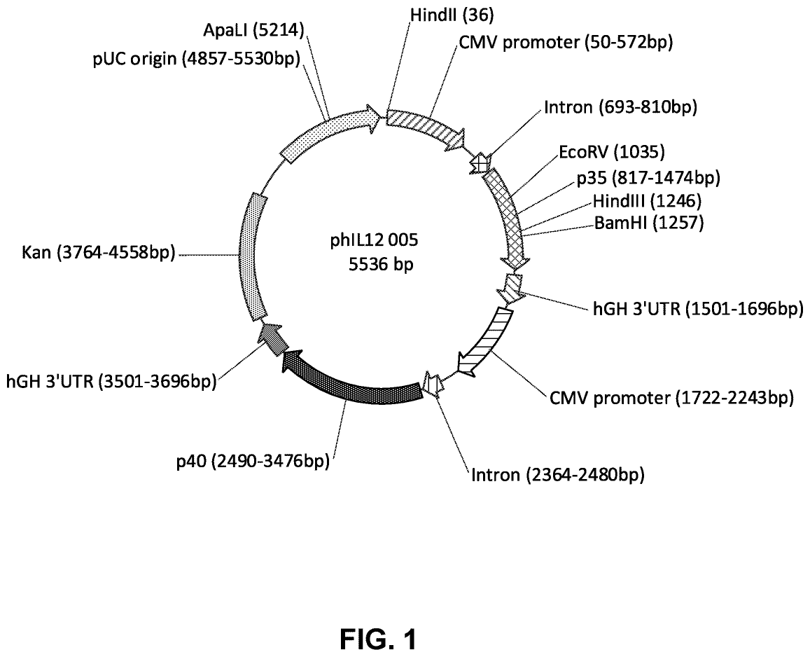

UTR (e.g., hGH 3′ UTR), an antibiotic resistance gene, or any combination thereof (e.g., the elements of FIG. 1).

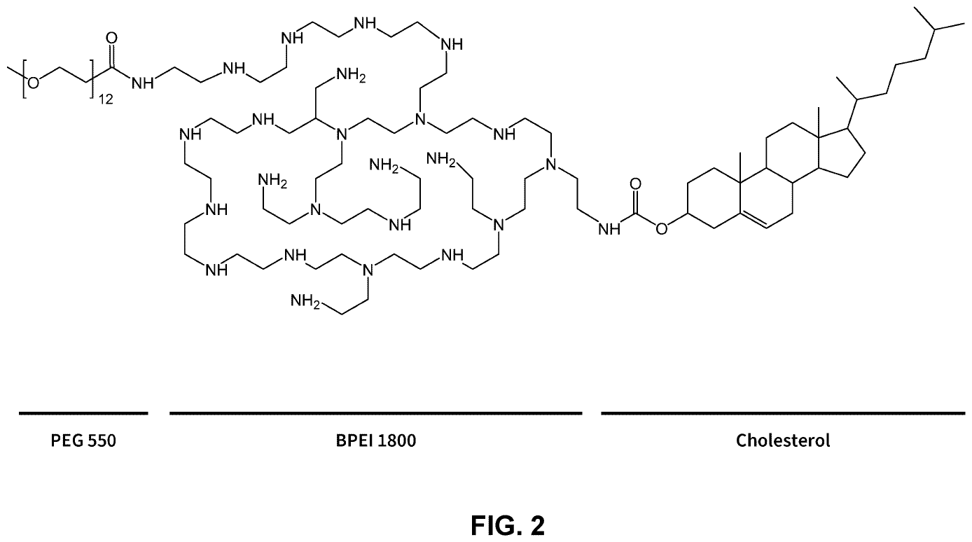

In some aspects, the lipopolymer comprises a polyethyleneimine (PEI) covalently linked independently to cholesterol and polyethylene glycol (PEG) groups (e.g., the lipopolymer of FIG. 2).

In some aspects, the nanoparticle disclosed herein comprises a DNA plasmid that encodes human IL-12.

In some aspects, wherein nanoparticle comprises a synthetic polymer facilitating plasmid delivery that is a lipopolymer.

In some aspects, the lipopolymer further comprises polyethyleneimine (PEI) covalently linked independently to cholesterol and polyethylene glycol (PEG) groups

In some aspects, the gene delivery polymer is a cationic polymer or a non-condensing polymer. The cationic polymer is selected from the group comprising polylysine, polyethylenimine, functionalized derivatives of polyethylenimine (PEI), polypropylenimine, aminoglycoside-polyamine, dideoxy-diamino-b-cyclodextrin, spermine and spermidine. One example of a cationic gene delivery polymer suitable for the present disclosure is a PEI derivative comprising a PEI backbone, a lipid, and a hydrophilic polymer spacer wherein the lipid is directly bound to the polyethylenimine backbone or covalently bound to the polyethylene glycol spacer, which in turn is bound, via a bio compatible bond, to the PEI.

The cationic gene delivery polymer of the present disclosure may further comprise a targeting moiety including antibodies or antibody fragments, cell receptors, growth factor receptors, cytokine receptors, folate, transferrin, epidermal growth factor (EGF), insulin, asialoorosomucoid, mannose-6-phosphate (monocytes), mannose (macrophage, some B cells), Lewis^{X} and sialyl Lewis^{X} (endothelial cells), N-acetyllactosamine (T cells), galactose (colon carcinoma cells), and thrombomodulin (mouse lung endothelial cells), fusogenic agents such as polymixin B and hemaglutinin HA2, lysosomotrophic agents, nucleus localization signals (NLS) such as T-antigen, and the like. Another gene delivery polymer is a non-condensing polymer selected from the group comprising polyvinylpyrrolidone, polyvinylalcohol, poly(lactide-co-glycolide) (PLGA) and triblock copolymers of PLGA and PEG. The gene delivery polymer may also be a non-condensing polymer. Examples of such non-condensing polymers include polyvinyl pyrollidone, polyvinyl alcohol, poloxamers, polyglutamate, gelatin, polyphosphoesters, silk-elastin-like hydrogels, agarose hydrogels, lipid microtubules, poly(lactide-co-glycolide) and polyethyleneglycol-linked poly(lactide-co-glycolide).

The gene delivery polymer is a cationic polymer or a non-condensing polymer. The cationic polymer is selected from the group comprising polylysine, polyethylenimine, functionalized derivatives of polyethylenimine, polypropylenimine, aminoglycoside-polyamine, dideoxy-diamino-b-cyclodextrin, spermine and spermidine. One example of a cationic gene delivery polymer suitable for the present invention is a polyethylenimine derivative comprising a polyethylenimine (PEI) backbone, a lipid, and a polyethylene glycol spacer wherein the lipid is directly bound to the polyethylenimine backbone or covalently bound to the polyethylene glycol spacer, which in turn is bound, via a biocompatible bond, to the PEI.

In some aspects, the gene delivery polymer comprises a lipopolyamine with the following formula:

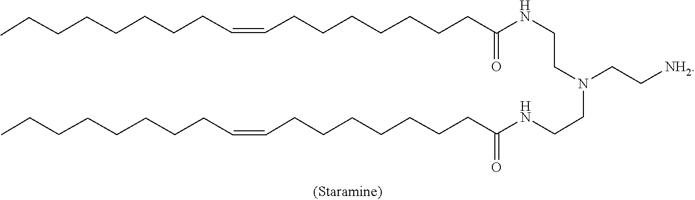 [SMILES: CCCCCCCC/C=C\CCCCCCCC(=O)NCCN(CCN)CCNC(=O)CCCCCCC/C=C\CCCCCCCC]

In some aspects, the gene delivery polymer comprises a mixture of the lipopolyamine and an alkylated derivative of the lipopolyamine. In some aspects, the alkylated derivative of the lipopolyamine is a polyoxyalkylene, polyvinylpyrrolidone, polyacrylamide, polydimethylacrylamide, polyvinyl alcohol, dextran, poly(L-glutamic acid), styrene maleic anhydride, poly-N-(2-hydroxypropyl) methacrylamide, or polydivinylether maleic anhydride. In some aspects, the alkylated derivative of the lipopolyamine has the following formula:

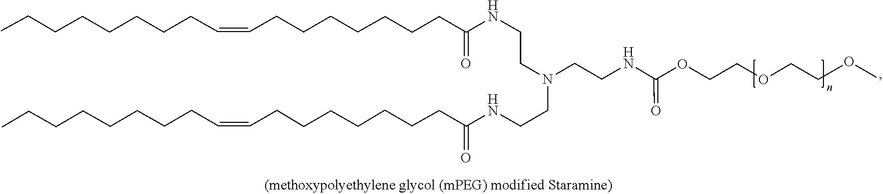

(methoxypolyethylene glycol (mPEG) modified Staramine),
wherein n represents an integer from 10 to 100 repeating units containing 2-5 carbon atoms each. In some aspects, the alkylated derivative of the lipopolyamine has the following formula:

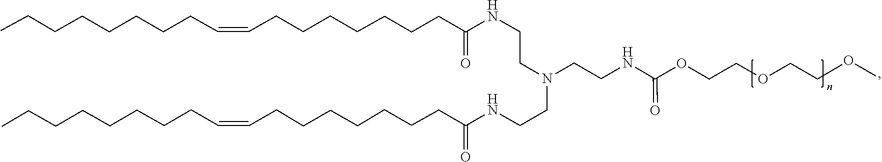

wherein n=11 (Staramine-mPEG515). In some aspects, the alkylated derivative of the lipopolyamine has the following formula:

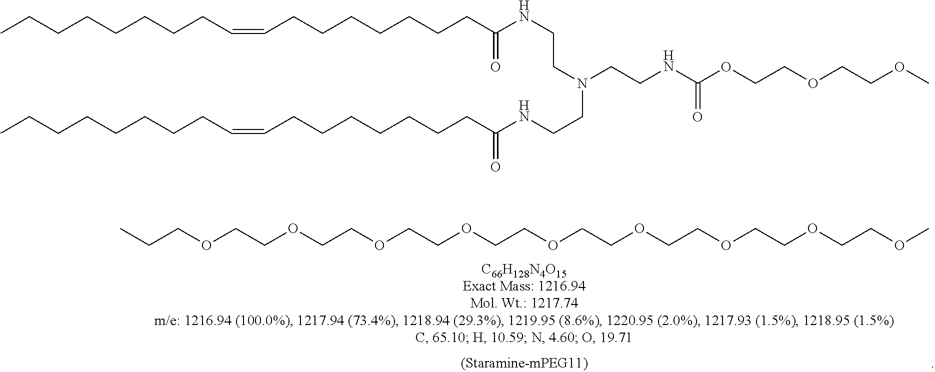

In some aspects, the ratio of the lipopolyamine to the alkylated derivative of the lipopolyamine in the mixture is 1:1 to 10:1. In some aspects, the lipopolyamine is present in an amount sufficient to produce a ratio of amine nitrogen in the lipopolyamine to phosphate in the nucleic acid vector from about 0.01:1 to about 50:1 (e.g., about 0.01:1 to about 40:1; about 0.01:1 to about 30:1; about 0.01:1 to about 20:1; about 0.01:1 to about 10:1, or about 0.01:1 to about 5:1). In some aspects, the ratio of amine nitrogen in the lipopolyamine to phosphate in the nucleic acid vector is from about 0.1:1 to about 50:1 (e.g., about 0.1:1 to about 40:1; about 0.1:1 to about 30:1; about 0.1:1 to about 20:1; about 0.1:1 to about 10:1, or about 0.1:1 to about 5:1). In some aspects, the ratio of amine nitrogen in the lipopolyamine to phosphate in the nucleic acid vector is from about 1:10 to about 10:1.

In some aspects, the gene delivery polymer comprises a lipopolyamine with the following formula:

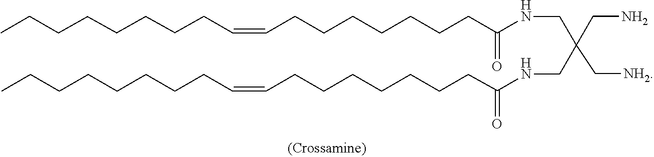 [SMILES: CCCCCCCC/C=C\CCCCCCCC(=O)NCC(CN)(CN)CNC(=O)CCCCCCC/C=C\CCCCCCCC]

In some aspects, the gene delivery polymer comprises a mixture of the lipopolyamine and an alkylated derivative of the lipopolyamine. In some aspects, the alkylated derivative of the lipopolyamine is a polyoxyalkylene, polyvinylpyrrolidone, polyacrylamide, polydimethylacrylamide, polyvinyl alcohol, dextran, poly(L-glutamic acid), styrene maleic anhydride, poly-N-(2-hydroxypropyl) methacrylamide, or polydivinylether maleic anhydride. In some aspects, the ratio of the lipopolyamine to the alkylated derivative of the lipopolyamine in the mixture is 1:1 to 10:1. In some aspects, the lipopolyamine is present in an amount sufficient to produce a ratio of amine nitrogen in the lipopolyamine to phosphate in the nucleic acid vector from about 0.01:1 to about 50:1 (e.g., about 0.01:1 to about 40:1; about 0.01:1 to about 30:1; about 0.01:1 to about 20:1; about 0.01:1 to about 10:1, or about 0.01:1 to about 5:1). In some aspects, the ratio of amine nitrogen in the lipopolyamine to phosphate in the nucleic acid vector is from about 0.1:1 to about 50:1 (e.g., about 0.1:1 to about 40:1; about 0.1:1 to about 30:1; about 0.1:1 to about 20:1; about 0.1:1 to about 10:1, or about 0.1:1 to about 5:1). In some aspects, the ratio of amine nitrogen in the lipopolyamine to phosphate in the nucleic acid vector is from about 1:10 to about 10:1.

In some aspects, the gene delivery polymer comprises a poloxamer back-bone having a metal chelator covalently coupled to at least one terminal end of the poloxamer backbone. In some aspects, the metal chelator is coupled to at least two terminal ends of the poloxamer backbone. In some aspects, the poloxamer backbone is a poloxamer backbone disclosed in U.S. Publ. No. 2010/0004313, which is herein incorporated by reference in its entirety. In some aspects, the metal chelator is a metal chelator disclosed in U.S. Publ. No. 2010/0004313. In some aspects, the gene delivery polymer comprises a polymer having the following formula:

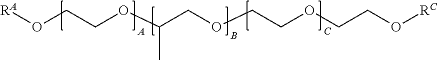

and pharmaceutically acceptable salts thereof, wherein:
A represents an integer from 2 to 141;
B represents an integer from 16 to 67;
C represents an integer from 2 to 141;
RA and RC are the same or different, and are R′-L- or H, wherein at least one of RA and RC is R′-L—;
L is a bond, —CO—, —CH2—O—, or —O—CO—; and
R′is a metal chelator.

In some aspects, the metal chelator is RNNH—, RN2N—, or (R″—(N(R″)—CH2CH2)x)2-N—CH2CO—, wherein each x is independently 0-2, and wherein R″ is HO2C—CH2—. In some aspects, the metal chelator is a crown ether selected from the group consisting of 12-crown-4, 15-crown-5, 18-crown-6, 20-crown-6, 21-crown-7, and 24-crown-8. In some aspects, the crown ether is a substituted-crown ether, wherein the substituted crown ether has:
(1) one or more of the crown ether oxygens independently replaced by NH or S,
(2) one or more of the crown ether-CH2-CH2-moieties replaced by—C6H4-, —C10H6—, or -C6H10-,
(3) one or more of the crown ether-CH2-O-CH2-moieties replaced by——C4H2O— or —C5H3N—, or
(4) any combination thereof.

In some aspects, the metal chelator is a cryptand, wherein the cryptand is selected from the group consisting of (1,2,2) cryptand, (2,2,2) cryptand, (2,2,3) cryptand, and (2,3,3) cryptand. In some aspects, the cryptand is a substituted-cryptand, wherein the substituted cryptand has:
(1) one or more of the crypthand ether oxygens independently replaced by NH or S,
(2) one or more of the crown ether-CH2-CH2-moieties replaced by—C6H4-, —C10H6—, or -C6H10-,
(3) one or more of the crown ether-CH2-O-CH2-moieties replaced by —C4H2O— or —C5H3N—, or
(4) any combination thereof.

In some aspects, the gene delivery polymer is Crown Poloxamer (aza-crown-linked poloxamer), wherein the Crown Poloxamer comprises a polymer having the following formula:

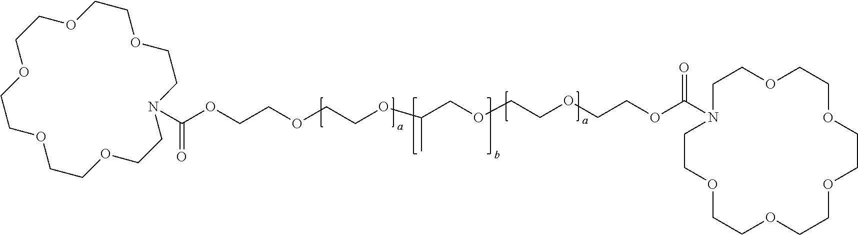

or pharmaceutically acceptable salts thereof, wherein:
a represents an integer of about 10 units; and
b represents an integer of about 21 units; and
wherein the total molecular weight of the polymer is about 2,000 Da to about 2,200 Da.

In some aspects, the gene delivery polymer is present in a solution with the nucleic acid vector from about 0.1%-about 5% or about 0.5%-about 5%.

In some aspects, the gene delivery polymer is a β-amino ester. In some aspects, the polymer is present in a solution with the nucleic acid vector from about 0.1%-about 5% or about 0.5%-about 5%.

In some aspects, the gene delivery polymer is a poly-inosinic-polycytidylic acid. In some aspects, the poly-inosinic-polycytidylic acid is present in a solution with the nucleic acid vector from about 0.1%-about 5% or about 0.5%-about 5%.

In some aspects, the gene delivery polymer further comprises benzalkonium chloride.

In some aspects, the gene delivery polymer comprises BD15-12. In some aspects, the ratio of nucleotide to BD15-12 polymer (N:P) is 5:1.

In some aspects, the gene delivery polymer comprises Omnifect. In some aspects, the ratio of nucleotide to Omnifect polymer (N:P) is 10:1.

In some aspects, the gene delivery polymer comprises Crown Poloxamer (aza-crown-linked poloxamer). In some aspects, the ratio of nucleotide to Crown Poloxamer (N:P) is 5:1. In some aspects, the gene delivery polymer comprises Crown Poloxamer and a PEG-PEI-cholesterol (PPC) lipopolymer. In some aspects, the gene delivery polymer comprises Crown Poloxamer and benzalkonium chloride. In some aspects, the gene delivery polymer comprises Crown Poloxamer and Omnifect. In some aspects, the gene delivery polymer comprises Crown Poloxamer and a linear polyethyleneimine (LPEI). In some aspects, the gene delivery polymer comprises Crown Poloxamer and BD15-12.

In some aspects, the gene delivery polymer comprises Staramine and mPEG modified Staramine. In some aspects, the mPEG modified Staramine is Staramine-mPEG515. In some aspects, the mPEG modified Staramine is Staramine-mPEG11. In some aspects, the ratio of Staramine to mPEG modified Staramine is 10:1. In some aspects, the nucleotide to polymer (N:P) ratio is 5:1. In some aspects, the gene delivery polymer comprises Staramine, mPEG modified Staramine, and Crown Poloxamer. In some aspects, the gene delivery polymer comprises Staramine, Staramine-mPEG515, and Crown Poloxamer. In some aspects, the gene delivery polymer comprises Staramine, Staramine-mPEG11, and Crown Poloxamer.

In some aspects, the gene delivery polymer comprises a poloxamer backbone disclosed in WO 2022/072910 A1, which is herein incorporated by reference in its entirety.

In some aspects, the nanoparticle that comprises a DNA plasmid that encodes interleukin-12 (IL-12) and a synthetic polymer facilitating plasmid delivery, is delivered intraperitoneally.

In some aspects, the nanoparticle is administered at a dose of about 35 mg/m² to about 80 mg/m². In some aspects, the nanoparticle is administered at a dose of about 40 mg/m² to about 80 mg/m². In some aspects, the nanoparticle is administered at a dose of about 45 mg/m² to about 80 mg/m². In some aspects, the nanoparticle is administered at a dose of about 50 mg/m² to about 80 mg/m². In some aspects, the nanoparticle is administered at a dose of about 55 mg/m² to about 80 mg/m². In some aspects, the nanoparticle is administered at a dose of about 60 mg/m² to about 80 mg/m². In some aspects, the nanoparticle is administered at a dose of about 65 mg/m² to about 80 mg/m². In some aspects, the nanoparticle is administered at a dose of about 70 mg/m² to about 80 mg/m². In some aspects, the nanoparticle is administered at a dose of about 75 mg/m² to about 80 mg/m². In some aspects, the nanoparticle is administered at a dose of about 35 mg/m² to about 75 mg/m². In some aspects, the nanoparticle is administered at a dose of about 35 mg/m² to about 70 mg/m². In some aspects, the nanoparticle is administered at a dose of about 35 mg/m² to about 65 mg/m². In some aspects, the nanoparticle is administered at a dose of about 35 mg/m² to about 60 mg/m². In some aspects, the nanoparticle is administered at a dose of about 35 mg/m² to about 55 mg/m². In some aspects, the nanoparticle is administered at a dose of about 35 mg/m² to about 50 mg/m². In some aspects, the nanoparticle is administered at a dose of about 35 mg/m² to about 45 mg/m². In some aspects, the nanoparticle is administered at a dose of about 35 mg/m² to about 40 mg/m².

In some aspects, the nanoparticle is administered at a dose of about 35 mg/m², about 40 mg/m², about 45 mg/m², about 50 mg/m², about 55 mg/m², about 60 mg/m², about 65 mg/m², about 70 mg/m², about 75 mg/m², or about 80 mg/m².

In some aspects, the nanoparticle is administered at a dose of about 60 mg/m².

### III. Immune Checkpoint Inhibitors

Immune checkpoint proteins interact with specific ligands which send a signal into T-cells that inhibits T-cell function. Cancer cells exploit this by driving high level expression of checkpoint proteins on their surface thereby suppressing the anti-cancer immune response.

An immune checkpoint inhibitor comprises any compound capable of inhibiting the function of an immune checkpoint protein. Inhibition includes reduction of function as well as full blockade. In some aspects, the immune checkpoint protein is a human checkpoint protein.

In some aspects, the immune checkpoint inhibitor is an antagonist of an immune checkpoint protein. In some aspects, the immune checkpoint inhibitor is an agonist of an immune checkpoint protein.

In some aspects, the immune checkpoint protein is selected from the group consisting of CTLA-4, PD-1 (and its ligands PD-L1 and PD-L2), B7-H3, B7-H4, HVEM, TIM3, GAL9, LAG-3, VISTA, KIR, BTLA, TIGIT, IDO-1, CEA, PVRIG, GARP, STING, Siglec-15, CD20, CD27, CD38, CD39, CD47, CD66a (CEACAM1), CD73, CD80, CD86, CD93, CD96, and/or CD161.

In some aspects, the immune checkpoint inhibitor is an inhibitor of an immune checkpoint protein selected from the group consisting of CTLA-4, PD-1 (and its ligands PD-L1 and PD-L2), and/or LAG-3.

In some aspects, the immune checkpoint inhibitor is a small molecule inhibitor.

In some aspects, the immune checkpoint inhibitor is an antibody or fragment thereof that specifically binds to PD-1. In some aspects, the immune checkpoint inhibitor is an antibody or fragment thereof that specifically binds to PD-L1. In some aspects, the immune checkpoint inhibitor is an antibody or fragment thereof that specifically binds to CTLA-4. In some aspects, the immune checkpoint inhibitor is an antibody or fragment thereof that specifically binds to LAG-3.

In some aspects, the immune checkpoint inhibitor is an antibody. In some aspects, immune checkpoint inhibitors comprise an antibody or fragment thereof that specifically bind to an immune checkpoint protein. In some aspects, the immune checkpoint inhibitor is a monoclonal antibody, a fully human antibody, a chimeric antibody, a humanized antibody or fragment thereof that capable of at least partly antagonizing an immune checkpoint protein.

In some aspects, the immune checkpoint inhibitor comprises a heavy chain variable region (VH) amino acid sequence and a light chain variable region (VL) amino acid sequence disclosed in Table 1. In some aspects, the immune checkpoint inhibitor comprises a heavy chain (HC) amino acid sequence and a light chain (LC) amino acid sequence disclosed in Table 2. In some aspects, the immune checkpoint inhibitor comprises a VH complementarity determining region (CDR) 1, a VH CDR2, a VH CDR3, a VL CDR1, a VL CDR2, and a VL CDR3 as disclosed in Table 3.

TABLE 1
Immune Checkpoint Inhibitor Antibody VH and VL Sequences

| SEQ ID NO: | Description(s) | Sequence(s) |
|---|---|---|
| 1 | nivolumab VH | QVQLVESGGGVVQPGRSLRLDCKASGITFSNSGMHWVRQA |
| | | PGKGLEWVAVIWYDGSKRYYADSVKGRFTISRDNSKNTLF |
| | | LQMNSLRAEDTAVYYCATNDDYWGQGTLVTVSS |
| 2 | nivolumab VL | EIVLTQSPATLSLSPGERATLSCRASQSVSSYLAWYQQKP |
| | | GQAPRLLIYDASNRATGIPARFSGSGSGTDFTLTISSLEP |
| | | EDFAVYYCQQSSNWPRTFGQGTKVEIK |
| 3 | pembrolizumab VH | QVQLVQSGVEVKKPGASVKVSCKASGYTFTNYYMYWVRQA |
| | | PGQGLEWMGGINPSNGGTNFNEKFKNRVTLTTDSSTTTAY |
| | | MELKSLQFDDTAVYYCARRDYRFDMGFDYWGQGTTVTVSS |
| 4 | pembrolizumab VL | EIVLTQSPATLSLSPGERATLSCRASKGVSTSGYSYLHWY |
| | | QQKPGQAPRLLIYLASYLESGVPARFSGSGSGTDFTLTIS |
| | | SLEPEDFAVYYCQHSRDLPLTFGGGTKVEIK |
| 5 | dostarlimab VH | EVQLLESGGGLVQPGGSLRLSCAASGFTFSSYDMSWVRQA |
| | | PGKGLEWVSTISGGGSYTYYQDSVKGRFTISRDNSKNTLY |
| | | LQMNSLRAEDTAVYYCASPYYAMDYWGQGTTVTVSS |
| 6 | dostarlimab VL | DIQLTQSPSFLSAYVGDRVTITCKASQDVGTAVAWYQQKP |
| | | GKAPKLLIYWASTLHTGVPSRFSGSGSGTEFTLTISSLQP |
| | | EDFATYYCQHYSSYPWTFGQGTKLEIK |
| 7 | cemiplimab VH | EVQLLESGGVLVQPGGSLRLSCAASGFTFSNFGMTWVRQA |
| | | PGKGLEWVSGISGGGRDTYFADSVKGRFTISRDNSKNTLY |
| | | LQMNSLKGEDTAVYYCVKWGNIYFDYWGQGTLVTVSS |
| 8 | cemiplimab VL | DIQMTQSPSSLSASVGDSITITCRASLSINTFLNWYQQKP |
| | | GKAPNLLIYAASSLHGGVPSRFSGSGSGTDFTLTIRTLQP |
| | | EDFATYYCQQSSNTPFTFGPGTVVDFR |
| 9 | atezolizumab VH | EVQLVESGGGLVQPGGSLRLSCAASGFTFSDSWIHWVRQA |
| | | PGKGLEWVAWISPYGGSTYYADSVKGRFTISADTSKNTAY |
| | | LQMNSLRAEDTAVYYCARRHWPGGFDYWGQGTLVTVSS |
| 10 | atezolizumab VL | DIQMTQSPSSLSASVGDRVTITCRASQDVSTAVAWYQQKP |
| | | GKAPKLLIYSASFLYSGVPSRFSGSGSGTDFTLTISSLQP |
| | | EDFATYYCQQYLYHPATFGQGTKVEIK |
| 11 | durvalumab VH | EVQLVESGGGLVQPGGSLRLSCAASGFTFSRYWMSWVRQA |
| | | PGKGLEWVANIKQDGSEKYYVDSVKGRFTISRDNAKNSLY |
| | | LQMNSLRAEDTAVYYCAREGGWFGELAFDYWGQGTLVTVS |
| | | S |
| 12 | durvalumab VL | EIVLTQSPGTLSLSPGERATLSCRASQRVSSSYLAWYQQK |
| | | PGQAPRLLIYDASSRATGIPDRFSGSGSGTDFTLTISRLE |
| | | PEDFAVYYCQQYGSLPWTFGQGTKVEIK |
| 13 | avelumab VH | EVQLLESGGGLVQPGGSLRLSCAASGFTFSSYIMMWVRQA |
| | | PGKGLEWVSSIYPSGGITFYADTVKGRFTISRDNSKNTLY |
| | | LQMNSLRAEDTAVYYCARIKLGTVTTVDYWGQGTLVTVSS |
| 14 | avelumab VL | QSALTQPASVSGSPGQSITISCTGTSSDVGGYNYVSWYQQ |
| | | HPGKAPKLMIYDVSNRPSGVSNRFSGSKSGNTASLTISGL |
| | | QAEDEADYYCSSYTSSSTRVFGTGTKVTVL |
| 15 | ipilimumab VH | QVQLVESGGGVVQPGRSLRLSCAASGFTFSSYTMHWVRQA |
| | | PGKGLEWVTFISYDGNNKYYADSVKGRFTISRDNSKNTLY |
| | | LQMNSLRAEDTAIYYCARTGWLGPFDYWGQGTLVTVSS |
| 16 | ipilimumab VL | EIVLTQSPGTLSLSPGERATLSCRASQSVGSSYLAWYQQK |
| | | PGQAPRLLIYGAFSRATGIPDRFSGSGSGTDFTLTISRLE |
| | | PEDFAVYYCQQYGSSPWTFGQGTKVEIK |
| 17 | relatlimab VH | QVQLQQWGAGLLKPSETLSLTCAVYGGSFSDYYWNWIRQP |
| | | PGKGLEWIGEINHRGSTNSNPSLKSRVTLSLDTSKNQFSL |
| | | KLRSVTAADTAVYYCAFGYSDYEYNWFDPWGQGTLVTVSS |
| 18 | relatlimab VL | EIVLTQSPATLSLSPGERATLSCRASQSISSYLAWYQQKP |
| | | GQAPRLLIYDASNRATGIPARFSGSGSGTDFTLTISSLEP |
| | | EDFAVYYCQQRSNWPLTFGQGTNLEIK |

TABLE 2
Immune Checkpoint Inhibitor Antibody HC and LC Sequences

| SEQ ID NO: | Description(s) | Sequence(s) |
|---|---|---|
| 19 | nivolumab HC | QVQLVESGGGVVQPGRSLRLDCKASGITFSNSGMHWVRQA |
| | | PGKGLEWVAVIWYDGSKRYYADSVKGRFTISRDNSKNTLF |
| | | LQMNSLRAEDTAVYYCATNDDYWGQGTLVTVSSASTKGPS |
| | | VFPLAPCSRSTSESTAALGCLVKDYFPEPVTVSWNSGALT |
| | | SGVHTFPAVLQSSGLYSLSSVVTVPSSSLGTKTYTCNVDH |
| | | KPSNTKVDKRVESKYGPPCPPCPAPEFLGGPSVFLFPPKP |
| | | KDTLMISRTPEVTCVVVDVSQEDPEVQFNWYVDGVEVHNA |
| | | KTKPREEQFNSTYRVVSVLTVLHQDWLNGKEYKCKVSNKG |
| | | LPSSIEKTISKAKGQPREPQVYTLPPSQEEMTKNQVSLTC |
| | | LVKGFYPSDIAVEWESNGQPENNYKTTPPVLDSDGSFFLY |
| | | SRLTVDKSRWQEGNVFSCSVMHEALHNHYTQKSLSLSLGK |
| 20 | nivolumab LC | EIVLTQSPATLSLSPGERATLSCRASQSVSSYLAWYQQKP |
| | | GQAPRLLIYDASNRATGIPARFSGSGSGTDFTLTISSLEP |
| | | EDFAVYYCQQSSNWPRTFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 21 | pembrolizumab HC | QVQLVQSGVEVKKPGASVKVSCKASGYTFTNYYMYWVRQA |
| | | PGQGLEWMGGINPSNGGTNFNEKFKNRVTLTTDSSTTTAY |
| | | MELKSLQFDDTAVYYCARRDYRFDMGFDYWGQGTTVTVSS |
| | | ASTKGPSVFPLAPCSRSTSESTAALGCLVKDYFPEPVTVS |
| | | WNSGALTSGVHTFPAVLQSSGLYSLSSVVTVPSSSLGTKT |
| | | YTCNVDHKPSNTKVDKRVESKYGPPCPPCPAPEFLGGPSV |
| | | FLFPPKPKDTLMISRTPEVTCVVVDVSQEDPEVQFNWYVD |
| | | GVEVHNAKTKPREEQFNSTYRVVSVLTVLHQDWLNGKEYK |
| | | CKVSNKGLPSSIEKTISKAKGQPREPQVYTLPPSQEEMTK |
| | | NQVSLTCLVKGFYPSDIAVEWESNGQPENNYKTTPPVLDS |
| | | DGSFFLYSRLTVDKSRWQEGNVFSCSVMHEALHNHYTQKS |
| | | LSLSLGK |
| 22 | pembrolizumab LC | EIVLTQSPATLSLSPGERATLSCRASKGVSTSGYSYLHWY |
| | | QQKPGQAPRLLIYLASYLESGVPARFSGSGSGTDFTLTIS |
| | | SLEPEDFAVYYCQHSRDLPLTFGGGTKVEIKRTVAAPSVF |
| | | IFPPSDEQLKSGTASVVCLLNNFYPREAKVQWKVDNALQS |
| | | GNSQESVTEQDSKDSTYSLSSTLTLSKADYEKHKVYACEV |
| | | THQGLSSPVTKSFNRGEC |
| 23 | dostarlimab HC | EVQLLESGGGLVQPGGSLRLSCAASGFTFSSYDMSWVRQA |
| | | PGKGLEWVSTISGGGSYTYYQDSVKGRFTISRDNSKNTLY |
| | | LQMNSLRAEDTAVYYCASPYYAMDYWGQGTTVTVSSASTK |
| | | GPSVFPLAPCSRSTSESTAALGCLVKDYFPEPVTVSWNSG |
| | | ALTSGVHTFPAVLQSSGLYSLSSVVTVPSSSLGTKTYTCN |
| | | VDHKPSNTKVDKRVESKYGPPCPPCPAPEFLGGPSVFLFP |
| | | PKPKDTLMISRTPEVTCVVVDVSQEDPEVQFNWYVDGVEV |
| | | HNAKTKPREEQFNSTYRVVSVLTVLHQDWLNGKEYKCKVS |
| | | NKGLPSSIEKTISKAKGQPREPQVYTLPPSQEEMTKNQVS |
| | | LTCLVKGFYPSDIAVEWESNGQPENNYKTTPPVLDSDGSF |
| | | FLYSRLTVDKSRWQEGNVFSCSVMHEALHNHYTQKSLSLS |
| | | LGK |
| 24 | dostarlimab LC | DIQLTQSPSFLSAYVGDRVTITCKASQDVGTAVAWYQQKP |
| | | GKAPKLLIYWASTLHTGVPSRFSGSGSGTEFTLTISSLQP |
| | | EDFATYYCQHYSSYPWTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 25 | cemiplimab HC | EVQLLESGGVLVQPGGSLRLSCAASGFTFSNFGMTWVRQA |
| | | PGKGLEWVSGISGGGRDTYFADSVKGRFTISRDNSKNTLY |
| | | LQMNSLKGEDTAVYYCVKWGNIYFDYWGQGTLVTVSSAST |
| | | KGPSVFPLAPCSRSTSESTAALGCLVKDYFPEPVTVSWNS |
| | | GALTSGVHTFPAVLQSSGLYSLSSVVTVPSSSLGTKTYTC |
| | | NVDHKPSNTKVDKRVESKYGPPCPPCPAPEFLGGPSVFLF |
| | | PPKPKDTLMISRTPEVTCVVVDVSQEDPEVQFNWYVDGVE |
| | | VHNAKTKPREEQFNSTYRVVSVLTVLHQDWLNGKEYKCKV |
| | | SNKGLPSSIEKTISKAKGQPREPQVYTLPPSQEEMTKNQV |
| | | SLTCLVKGFYPSDIAVEWESNGQPENNYKTTPPVLDSDGS |
| | | FFLYSRLTVDKSRWQEGNVFSCSVMHEALHNHYTQKSLSL |
| | | SLGK |
| 26 | cemiplimab LC | DIQMTQSPSSLSASVGDSITITCRASLSINTFLNWYQQKP |
| | | GKAPNLLIYAASSLHGGVPSRFSGSGSGTDFTLTIRTLQP |
| | | EDFATYYCQQSSNTPFTFGPGTVVDFRRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 27 | atezolizumab HC | EVQLVESGGGLVQPGGSLRLSCAASGFTFSDSWIHWVRQA |
| | | PGKGLEWVAWISPYGGSTYYADSVKGRFTISADTSKNTAY |
| | | LQMNSLRAEDTAVYYCARRHWPGGFDYWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKKVEPKSCDKTHTCPPCPAPELLGGPS |
| | | VFLFPPKPKDTLMISRTPEVTCVVVDVSHEDPEVKFNWYV |
| | | DGVEVHNAKTKPREEQYASTYRVVSVLTVLHQDWLNGKEY |
| | | KCKVSNKALPAPIEKTISKAKGQPREPQVYTLPPSREEMT |
| | | KNQVSLTCLVKGFYPSDIAVEWESNGQPENNYKTTPPVLD |
| | | SDGSFFLYSKLTVDKSRWQQGNVFSCSVMHEALHNHYTQK |
| | | SLSLSPGK |
| 28 | atezolizumab LC | DIQMTQSPSSLSASVGDRVTITCRASQDVSTAVAWYQQKP |
| | | GKAPKLLIYSASFLYSGVPSRFSGSGSGTDFTLTISSLQP |
| | | EDFATYYCQQYLYHPATFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 29 | durvalumab HC | EVQLVESGGGLVQPGGSLRLSCAASGFTFSRYWMSWVRQA |
| | | PGKGLEWVANIKQDGSEKYYVDSVKGRFTISRDNAKNSLY |
| | | LQMNSLRAEDTAVYYCAREGGWFGELAFDYWGQGTLVTVS |
| | | SASTKGPSVFPLAPSSKSTSGGTAALGCLVKDYFPEPVTV |
| | | SWNSGALTSGVHTFPAVLQSSGLYSLSSVVTVPSSSLGTQ |
| | | TYICNVNHKPSNTKVDKRVEPKSCDKTHTCPPCPAPEFEG |
| | | GPSVFLFPPKPKDTLMISRTPEVTCVVVDVSHEDPEVKFN |
| | | WYVDGVEVHNAKTKPREEQYNSTYRVVSVLTVLHQDWING |
| | | KEYKCKVSNKALPASIEKTISKAKGQPREPQVYTLPPSRE |
| | | EMTKNQVSLTCLVKGFYPSDIAVEWESNGQPENNYKTTPP |
| | | VLDSDGSFFLYSKLTVDKSRWQQGNVFSCSVMHEALHNHY |
| | | TQKSLSLSPGK |
| 30 | durvalumab LC | EIVLTQSPGTLSLSPGERATLSCRASQRVSSSYLAWYQQK |
| | | PGQAPRLLIYDASSRATGIPDRFSGSGSGTDFTLTISRLE |
| | | PEDFAVYYCQQYGSLPWTFGQGTKVEIKRTVAAPSVFIFP |
| | | PSDEQLKSGTASVVCLLNNFYPREAKVQWKVDNALQSGNS |
| | | QESVTEQDSKDSTYSLSSTLTLSKADYEKHKVYACEVTHQ |
| | | GLSSPVTKSFNRGEC |
| 31 | avelumab HC | EVQLLESGGGLVQPGGSLRLSCAASGFTFSSYIMMWVRQA |
| | | PGKGLEWVSSIYPSGGITFYADTVKGRFTISRDNSKNTLY |
| | | LQMNSLRAEDTAVYYCARIKLGTVTTVDYWGQGTLVTVSS |
| | | ASTKGPSVFPLAPSSKSTSGGTAALGCLVKDYFPEPVTVS |
| | | WNSGALTSGVHTFPAVLQSSGLYSLSSVVTVPSSSLGTQT |
| | | YICNVNHKPSNTKVDKKVEPKSCDKTHTCPPCPAPELLGG |
| | | PSVFLFPPKPKDTLMISRTPEVTCVVVDVSHEDPEVKFNW |
| | | YVDGVEVHNAKTKPREEQYNSTYRVVSVLTVLHQDWLNGK |
| | | EYKCKVSNKALPAPIEKTISKAKGQPREPQVYTLPPSRDE |
| | | LTKNQVSLTCLVKGFYPSDIAVEWESNGQPENNYKTTPPV |
| | | LDSDGSFFLYSKLTVDKSRWQQGNVFSCSVMHEALHNHYT |
| | | QKSLSLSPGK |
| 32 | avelumab LC | QSALTQPASVSGSPGQSITISCTGTSSDVGGYNYVSWYQQ |
| | | HPGKAPKLMIYDVSNRPSGVSNRFSGSKSGNTASLTISGL |
| | | QAEDEADYYCSSYTSSSTRVFGTGTKVTVLGQPKANPTVT |
| | | LFPPSSEELQANKATLVCLISDFYPGAVTVAWKADGSPVK |
| | | AGVETTKPSKQSNNKYAASSYLSLTPEQWKSHRSYSCQVT |
| | | HEGSTVEKTVAPTECS |
| 33 | ipilimumab HC | QVQLVESGGGVVQPGRSLRLSCAASGFTFSSYTMHWVRQA |
| | | PGKGLEWVTFISYDGNNKYYADSVKGRFTISRDNSKNTLY |
| | | LQMNSLRAEDTAIYYCARTGWLGPFDYWGQGTLVTVSSAS |
| | | TKGPSVFPLAPSSKSTSGGTAALGCLVKDYFPEPVTVSWN |
| | | SGALTSGVHTFPAVLQSSGLYSLSSVVTVPSSSLGTQTYI |
| | | CNVNHKPSNTKVDKRVEPKSCDKTHTCPPCPAPELLGGPS |
| | | VFLFPPKPKDTLMISRTPEVTCVVVDVSHEDPEVKFNWYV |
| | | DGVEVHNAKTKPREEQYNSTYRVVSVLTVLHQDWLNGKEY |
| | | KCKVSNKALPAPIEKTISKAKGQPREPQVYTLPPSRDELT |
| | | KNQVSLTCLVKGFYPSDIAVEWESNGQPENNYKTTPPVLD |
| | | SDGSFFLYSKLTVDKSRWQQGNVFSCSVMHEALHNHYTQK |
| | | SLSLSPGK |
| 34 | ipilimumab LC | EIVLTQSPGTLSLSPGERATLSCRASQSVGSSYLAWYQQK |
| | | PGQAPRLLIYGAFSRATGIPDRFSGSGSGTDFTLTISRLE |
| | | PEDFAVYYCQQYGSSPWTFGQGTKVEIKRTVAAPSVFIFP |
| | | PSDEQLKSGTASVVCLLNNFYPREAKVQWKVDNALQSGNS |
| | | QESVTEQDSKDSTYSLSSTLTLSKADYEKHKVYACEVTHQ |
| | | GLSSPVTKSFNRGEC |
| 35 | relatlimab HC | QVQLQQWGAGLLKPSETLSLTCAVYGGSFSDYYWNWIRQP |
| | | PGKGLEWIGEINHRGSTNSNPSLKSRVTLSLDTSKNQFSL |
| | | KLRSVTAADTAVYYCAFGYSDYEYNWFDPWGQGTLVTVSS |
| | | ASTKGPSVFPLAPCSRSTSESTAALGCLVKDYFPEPVTVS |
| | | WNSGALTSGVHTFPAVLQSSGLYSLSSVVTVPSSSLGTKT |
| | | YTCNVDHKPSNTKVDKRVESKYGPPCPPCPAPEFLGGPSV |
| | | FLFPPKPKDTLMISRTPEVTCVVVDVSQEDPEVQFNWYVD |
| | | GVEVHNAKTKPREEQFNSTYRVVSVLTVLHQDWLNGKEYK |
| | | CKVSNKGLPSSIEKTISKAKGQPREPQVYTLPPSQEEMTK |
| | | NQVSLTCLVKGFYPSDIAVEWESNGQPENNYKTTPPVLDS |
| | | DGSFFLYSRLTVDKSRWQEGNVFSCSVMHEALHNHYTQKS |
| | | LSLSLG |
| 36 | relatlimab LC | EIVLTQSPATLSLSPGERATLSCRASQSISSYLAWYQQKP |
| | | GQAPRLLIYDASNRATGIPARFSGSGSGTDFTLTISSLEP |
| | | EDFAVYYCQQRSNWPLTFGQGTNLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |

TABLE 3
Immune Checkpoint Inhibitor Antibody
VH CDR and VL CDR Sequences

| SEQ ID NO: | Description(s) | Sequence(s) |
|---|---|---|
| 37 | nivolumab | GITFSNSG |
| | VH CDR1 | |
| 38 | nivolumab | IWYDGSKR |
| | VH CDR2 | |
| 39 | nivolumab | ATNDDY |
| | VH CDR3 | |
| 40 | nivolumab | QSVSSY |
| | VL CDR1 | |
| — | nivolumab | DAS |
| | VL CDR2 | |
| 41 | nivolumab | QQSSNWPRT |
| | VL CDR3 | |
| 42 | pembrolizumab | GYTFTNYY |
| | VH CDR1 | |
| 43 | pembrolizumab | INPSNGGT |
| | VH CDR2 | |
| 44 | pembrolizumab | ARRDYRFDMGFDY |
| | VH CDR3 | |
| 45 | pembrolizumab | KGVSTSGYSY |
| | VL CDR1 | |
| — | pembrolizumab | LAS |
| | VL CDR2 | |
| 46 | pembrolizumab | QHSRDLPLT |
| | VL CDR3 | |
| 47 | dostarlimab | GFTFSSYD |
| | VH CDR1 | |
| 48 | dostarlimab | ISGGGSYT |
| | VH CDR2 | |
| 49 | dostarlimab | ASPYYAMDY |
| | VH CDR3 | |
| 50 | dostarlimab | QDVGTA |
| | VL CDR1 | |
| — | dostarlimab | WAS |
| | VL CDR2 | |
| 51 | dostarlimab | QHYSSYPWT |
| | VL CDR3 | |
| 52 | cemiplimab | GFTFSNFG |
| | VH CDR1 | |
| 53 | cemiplimab | ISGGGRDT |
| | VH CDR2 | |
| 54 | cemiplimab | VKWGNIYFDY |
| | VH CDR3 | |
| 55 | cemiplimab | LSINTF |
| | VL CDR1 | |
| — | cemiplimab | AAS |
| | VL CDR2 | |
| 56 | cemiplimab | QQSSNTPFT |
| | VL CDR3 | |
| 57 | atezolizumab | GFTFSDSW |
| | VH CDR1 | |
| 58 | atezolizumab | ISPYGGST |
| | VH CDR2 | |
| 59 | atezolizumab | ARRHWPGGFDY |
| | VH CDR3 | |
| 60 | atezolizumab | QDVSTA |
| | VL CDR1 | |
| — | atezolizumab | SAS |
| | VL CDR2 | |
| 61 | atezolizumab | QQYLYHPAT |
| | VL CDR3 | |
| 62 | durvalumab | GFTFSRYW |
| | VH CDR1 | |
| 63 | durvalumab | IKQDGSEK |
| | VH CDR2 | |
| 64 | durvalumab | AREGGWFGELAFDY |
| | VH CDR3 | |
| 65 | durvalumab | QRVSSSY |
| | VL CDR1 | |
| — | durvalumab | DAS |
| | VL CDR2 | |
| 66 | durvalumab | QQYGSLPWT |
| | VL CDR3 | |
| 67 | avelumab | GFTFSSYI |
| | VH CDR1 | |
| 68 | avelumab | IYPSGGIT |
| | VH CDR2 | |
| 69 | avelumab | ARIKLGTVTTVDY |
| | VH CDR3 | |
| 70 | avelumab | SSDVGGYNY |
| | VL CDR1 | |
| — | avelumab | DVS |
| | VL CDR2 | |
| 71 | avelumab | SSYTSSSTRV |
| | VL CDR3 | |
| 72 | ipilimumab | GFTFSSYT |
| | VH CDR1 | |
| 73 | ipilimumab | ISYDGNNK |
| | VH CDR2 | |
| 74 | ipilimumab | ARTGWLGPFDY |
| | VH CDR3 | |
| 75 | ipilimumab | QSVGSSY |
| | VL CDR1 | |
| — | ipilimumab | GAF |
| | VL CDR2 | |
| 76 | ipilimumab | QQYGSSPWT |
| | VL CDR3 | |
| 77 | relatlimab | GGSFSDYY |
| | VH CDR1 | |
| 78 | relatlimab | INHRGST |
| | VH CDR2 | |
| 79 | relatlimab | AFGYSDYEYNWFDP |
| | VH CDR3 | |
| 80 | relatlimab | QSISSY |
| | VL CDR1 | |
| — | relatlimab | DAS |
| | VL CDR2 | |
| 81 | relatlimab | QQRSNWPLT |
| | VL CDR3 | |

In some aspects, the immune checkpoint inhibitor is ipilimumab. In some aspects, ipilimumab is administered at a dose of about 0.5 to about 5 mg/kg. In some aspects, ipilimumab is administered at a dose of about 1 to about 5 mg/kg. In some aspects, ipilimumab is administered at a dose of about 1.5 to about 5 mg/kg. In some aspects, ipilimumab is administered at a dose of about 2 to about 5 mg/kg. In some aspects, ipilimumab is administered at a dose of about 2.5 to about 5 mg/kg. In some aspects, ipilimumab is administered at a dose of about 3 to about 5 mg/kg. In some aspects, ipilimumab is administered at a dose of about 3.5 to about 5 mg/kg. In some aspects, ipilimumab is administered at a dose of about 4 to about 5 mg/kg. In some aspects, ipilimumab is administered at a dose of about 0.5 to about 4 mg/kg. In some aspects, ipilimumab is administered at a dose of about 0.5 to about 3 mg/kg. In some aspects, ipilimumab is administered at a dose of about 0.5 to about 2.5 mg/kg. In some aspects, ipilimumab is administered at a dose of about 0.5 to about 2 mg/kg. In some aspects, ipilimumab is administered at a dose of about 0.5 to about 1.5 mg/kg.

In some aspects, ipilimumab is administered at a dose of about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, or about 5 mg/kg.

In some aspects, ipilimumab is administered at a dose of about 1 mg/kg.

In some aspects, ipilimumab is administered every 1 week, 2 weeks, 3 weeks, 4 weeks, 5 weeks, 6 weeks, 7 weeks, or 8 weeks.

In some aspects, ipilimumab is administered every 2-8 weeks (e.g., every 6 weeks) during treatment.

In some aspects, ipilimumab is administered intravenously.

In some aspects, the immune checkpoint inhibitor is nivolumab. In some aspects, nivolumab is administered at a dose of about 120 mg to about 360 mg. In some aspects, nivolumab is administered at a dose of about 140 mg to about 360 mg. In some aspects, nivolumab is administered at a dose of about 160 mg to about 360 mg. In some aspects, nivolumab is administered at a dose of about 180 mg to about 360 mg. In some aspects, nivolumab is administered at a dose of about 200 mg to about 360 mg. In some aspects, nivolumab is administered at a dose of about 220 mg to about 360 mg. In some aspects, nivolumab is administered at a dose of about 240 mg to about 360 mg. In some aspects, nivolumab is administered at a dose of about 260 mg to about 360 mg. In some aspects, nivolumab is administered at a dose of about 280 mg to about 360 mg. In some aspects, nivolumab is administered at a dose of about 300 mg to about 360 mg. In some aspects, nivolumab is administered at a dose of about 320 mg to about 360 mg. In some aspects, nivolumab is administered at a dose of about 340 mg to about 360 mg. In some aspects, nivolumab is administered at a dose of about 120 mg to about 340 mg. In some aspects, nivolumab is administered at a dose of about 120 mg to about 320 mg. In some aspects, nivolumab is administered at a dose of about 120 mg to about 300 mg. In some aspects, nivolumab is administered at a dose of about 120 mg to about 280 mg. In some aspects, nivolumab is administered at a dose of about 120 mg to about 260 mg. In some aspects, nivolumab is administered at a dose of about 120 mg to about 240 mg. In some aspects, nivolumab is administered at a dose of about 120 mg to about 220 mg. In some aspects, nivolumab is administered at a dose of about 120 mg to about 200 mg. In some aspects, nivolumab is administered at a dose of about 120 mg to about 180 mg. In some aspects, nivolumab is administered at a dose of about 120 mg to about 160 mg. In some aspects, nivolumab is administered at a dose of about 120 mg to about 140 mg. In some aspects, nivolumab is administered at a dose of about 240 mg to about 360 mg. In some aspects, nivolumab is administered at a dose of about 260 mg to about 360 mg. In some aspects, nivolumab is administered at a dose of about 280 mg to about 360 mg. In some aspects, nivolumab is administered at a dose of about 300 mg to about 360 mg. In some aspects, nivolumab is administered at a dose of about 320 mg to about 360 mg. In some aspects, nivolumab is administered at a dose of about 340 mg to about 360 mg. In some aspects, nivolumab is administered at a dose of about 140 mg to about 240 mg. In some aspects, nivolumab is administered at a dose of about 160 mg to about 240 mg. In some aspects, nivolumab is administered at a dose of about 180 mg to about 240 mg. In some aspects, nivolumab is administered at a dose of about 200 mg to about 240 mg. In some aspects, nivolumab is administered at a dose of about 220 mg to about 240 mg.

In some aspects, nivolumab is administered at a dose of about 120 mg, about 140 mg, about 160 mg, about 180 mg, about 200 mg, about 220 mg, about 240 mg, about 260 mg, about 280 mg, about 300 mg, about 320 mg, about 340 mg, or about 360 mg.

In some aspects, nivolumab is administered at a dose of about 240 mg.

In some aspects, nivolumab is administered intravenously.

In some aspects, nivolumab is administered every 1 week, 2 weeks, 3 weeks, 4 weeks, 5 weeks, 6 weeks, 7 weeks, or 8 weeks.

In some aspects, nivolumab is administered every 1-4 weeks (e.g., every 2 weeks) during treatment.

### PD-1

Human monoclonal antibodies (HuMAbs) that bind specifically to PD-1 with high affinity have been disclosed in U.S. Pat. Nos. 8,008,449 and 8,779,105. Other anti-PD-1 mAbs have been described in, for example, U.S. Pat. Nos. 6,808,710, 7,488,802, 8,168,757 and 8,354,509, and PCT Publication No. WO 2012/145493. Each of the anti-PD-1 HuMAbs disclosed in U.S. Pat. No. 8,008,449 has been demonstrated to exhibit one or more of the following characteristics: (a) binds to human PD-1 with a K_{D} of 1×10⁻⁷ M or less, as determined by surface plasmon resonance using a Biacore biosensor system; (b) does not substantially bind to human CD28, CTLA-4 or ICOS; (c) increases T-cell proliferation in a Mixed Lymphocyte Reaction (MLR) assay; (d) increases interferon-γ production in an MLR assay; (e) increases IL-2 secretion in an MLR assay; (f) binds to human PD-1 and cynomolgus monkey PD-1; (g) inhibits the binding of PD-L1 and/or PD-L2 to PD-1; (h) stimulates antigen-specific memory responses; (i) stimulates Ab responses; and (j) inhibits tumor cell growth in vivo. In some aspects the anti-PD-1 antibodies of the combination therapy disclosed herein include mAbs that bind specifically to human PD-1 and exhibit at least one of the preceding characteristics.

In some aspects, the anti-PD-1 antibody is nivolumab. Nivolumab (also known as “OPDIVO®”; BMS-936558; formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2 (9): 846-56). In some aspects, the anti-PD-1 antibody or fragment thereof binds to the same epitope as nivolumab. In some aspects, the anti-PD-1 antibody has the same CDRs as nivolumab.

Anti-PD-1 antibodies useful for the disclosed compositions also include isolated antibodies that bind specifically to human PD-1 and cross-compete for binding to human PD-1 with nivolumab (see, e.g., U.S. Pat. Nos. 8,008,449 and 8,779,105; Int'l Pub. No. WO 2013/173223). The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar to those of nivolumab by virtue of their binding to the same epitope region of PD-1. Cross-competing antibodies can be readily identified based on their ability to cross-compete with nivolumab in standard PD-1 binding assays such as Biacore analysis, ELISA assays or flow cytometry (see, e.g., Int'l Pub. No. WO 2013/173223).

In certain embodiments, antibodies or antigen binding fragments thereof that cross-compete for binding to human PD-1 with, or bind to the same epitope region of human PD-1 as, nivolumab are mAbs. For administration to human subjects, these cross-competing antibodies can be chimeric antibodies, or humanized or human antibodies. Such chimeric, humanized or human mAbs can be prepared and isolated by methods well known in the art.

In some aspects, the anti-PD-1 antibody is pembrolizumab. Pembrolizumab (also known as “KEYTRUDA®”, lambrolizumab, and MK-3475) is a humanized monoclonal IgG4 (S228P) antibody directed against human cell surface receptor PD-1 (programmed death-1 or programmed cell death-1). Pembrolizumab is described, for example, in U.S. Pat. Nos. 8,354,509 and 8,900,587. Pembrolizumab has been approved by the FDA for the treatment of relapsed or refractory melanoma.

In some aspects, the anti-PD-1 antibody is REGN2810. In some aspects, the anti-PD-1 antibody is PDR001. Another known anti-PD-1 antibody is pidilizumab (CT-011). In some aspects, the anti-PD-1 antibody is MEDI0608 (formerly AMP-514), which is a monoclonal antibody. MEDI0608 is described, for example, in U.S. Pat. No. 8,609,089. In some aspects, the anti-PD-1 antibody or antigen binding fragment thereof is BGB-A317, which is a humanized monoclonal antibody. BGB-A317 is described in U.S. Publ. No. 2015/0079109.nnAntibodies or antigen binding fragments thereof that bind the same epitopes or have the same CDRs as any of these antibodies can be used.

Anti-human-PD-1 antibodies (or VH and/or VL domains derived therefrom) suitable for uses disclosed herein can be generated using methods well known in the art. Alternatively, art recognized anti-PD-1 antibodies can be used.

In aspects, the anti-PD-1 antibody is selected from the group consisting of nivolumab (also known as OPDIVO®, 5C4, BMS-936558, MDX-1106, and ONO-4538), pembrolizumab (Merck; also known as KEYTRUDA®, lambrolizumab, and MK-3475; see WO2008/156712), PDR001 (Novartis; see WO 2015/112900), MEDI-0680 (AstraZeneca; also known as AMP-514; see WO 2012/145493), cemiplimab (Regeneron; also known as REGN-2810; see WO 2015/112800), JS001 (TAIZHOU JUNSHI PHARMA; see Si-Yang Liu et al., J. Hematol. Oncol. 10:136 (2017)), BGB-A317 (Beigene; see WO 2015/35606 and US 2015/0079109), INCSHR1210 (Jiangsu Hengrui Medicine; also known as SHR-1210; see WO 2015/085847; Si-Yang Liu et al., J. Hematol. Oncol. 10:136 (2017)), TSR-042 (Tesaro Biopharmaceutical; also known as ANB011; see WO2014/179664), GLS-010 (Wuxi/Harbin Gloria Pharmaceuticals; also known as WBP3055; see Si-Yang Liu et al., J. Hematol. Oncol. 10:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), and IBI308 (Innovent; see WO 2017/024465, WO 2017/025016, WO 2017/132825, and WO 2017/133540), which references are herein incorporated by reference.

Other anti-PD-1 monoclonal antibodies have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540, each of which are herein incorporated by reference.

Anti-PD-1 antibodies useful for the combination therapy of the disclosed invention also include antigen-binding portions of the above antibodies. It has been amply demonstrated that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′) 2 fragment, a bivalent fragment comprising two Fab fragments linked by a disulfide bridge at the hinge region; (iii) a Fd fragment consisting of the VH and CH1 domains; and (iv) a Fv fragment consisting of the VL and VH domains of a single arm of an antibody.

Anti-PD-1 antibodies suitable for use in the disclosed combination therapies are antibodies that bind to PD-1 with high specificity and affinity, block the binding of PD-L1 and or PD-L2, and inhibit the immunosuppressive effect of the PD-1 signaling pathway. In certain embodiments, the anti-PD-1 antibody or antigen-binding portion thereof cross-competes with nivolumab for binding to human PD-1. In some aspects, the anti-PD-1 antibody or antigen-binding portion thereof is a chimeric, humanized or human monoclonal antibody or a portion thereof. In some aspects, the antibody is a humanized antibody. In other embodiments, the antibody is a human antibody. Antibodies of an IgG1, IgG2, IgG3 or IgG4 isotype can be used.

In some aspects, the anti-PD-1 antibody or antigen binding fragment thereof comprises a heavy chain constant region which is of a human IgG1 or IgG4 isotype. In some aspects, the sequence of the IgG4 heavy chain constant region of the anti-PD-1 antibody or antigen binding fragment thereof contains an S228P mutation which replaces a serine residue in the hinge region with the proline residue normally found at the corresponding position in IgG1 isotype antibodies. This mutation, which is present in nivolumab, prevents Fab arm exchange with endogenous IgG4 antibodies, while retaining the low affinity for activating Fc receptors associated with wild-type IgG4 antibodies (Wang et al., 2014). In some aspects, the antibody comprises a light chain constant region which is a human kappa or lambda constant region. In other embodiments, the anti-PD-1 antibody or antigen binding fragment thereof is a mAb or an antigen-binding portion thereof. In certain embodiments of any of the therapeutic methods described herein comprising administration of an anti-PD-1 antibody, the anti-PD-1 antibody is nivolumab.

In some aspects, the PD-1 antagonist is selected from the group consisting of nivolumab, pembrolizumab, cemiplimab, and dostarlimab.

### PD-L1

Anti-human-PD-L1 antibodies (or VH and/or VL domains derived therefrom) suitable for use in the combination therapy disclosed herein can be generated using methods known in the art. Examples of anti-PD-L1 antibodies useful in the methods of the present disclosure include the antibodies disclosed in U.S. Pat. No. 9,580,507, incorporated herein by reference. Anti-PD-L1 human monoclonal antibodies disclosed in U.S. Pat. No. 9,580,507 have been demonstrated to exhibit one or more of the following characteristics: (a) bind to human PD-L1 with a K_{D} of 1×10⁻⁷ M or less, as determined by surface plasmon resonance using a Biacore biosensor system; (b) increase T-cell proliferation in a Mixed Lymphocyte Reaction (MLR) assay; (c) increase interferon-γ production in an MLR assay; (d) increase IL-2 secretion in an MLR assay; (e) stimulate antibody responses; and (f) reverse the effect of T regulatory cells on T cell effector cells and/or dendritic cells. Anti-PD-L1 antibodies useful in the methods disclosed herein include monoclonal antibodies that bind specifically to human PD-L1 and exhibit at least one of the preceding characteristics.

A recognized anti-PD-L1 antibodies can be used. For example, human anti-PD-L1 antibodies disclosed in U.S. Pat. No. 7,943,743, the contents of which are hereby incorporated by reference, can be used. Such anti-PD-L1 antibodies include 3G10, 12A4 (also referred to as BMS-936559), 10A5, 5F8, 10H10, 1B12, 7H1, 11E6, 12B7, and 13G4. Other art recognized anti-PD-L1 antibodies which can be used include those described in, for example, U.S. Pat. Nos. 7,635,757 and 8,217,149, U.S. Publication No. 2009/0317368, and PCT Publication Nos. WO 2011/066389 and WO 2012/145493, the teachings of which also are hereby incorporated by reference. Other examples of an anti-PD-L1 antibody include atezolizumab (TECENTRIQ; RG7446), or durvalumab (IMFINZI; MEDI4736) or avelumab (Bavencio). Antibodies or antigen binding fragments thereof that compete with any of these art-recognized antibodies or inhibitors for binding to PD-L1 also can be used.

In some aspects, the anti-PD-L1 antibody is BMS-936559 (formerly 12A4 or MDX-1105) (see, e.g., U.S. Pat. No. 7,943,743; WO 2013/173223). In other embodiments, the anti-PD-L1 antibody is MPDL3280A (also known as RG7446 and atezolizumab) (see, e.g., Herbst et al. 2013 J Clin Oncol 31 (suppl): 3000; U.S. Pat. No. 8,217,149), MEDI4736 (Khleif, 2013, In: Proceedings from the European Cancer Congress 2013; Sep. 27-Oct. 1, 2013; Amsterdam, The Netherlands. Abstract 802), or MSB0010718C (also called Avelumab; see US 2014/0341917). In some aspects, antibodies that cross-compete for binding to human PD-L1 with, or bind to the same epitope region of human PD-L1 as the above-references PD-L1 antibodies are mAbs. For administration to human subjects, these cross-competing antibodies can be chimeric antibodies, or can be humanized or human antibodies. Such chimeric, humanized or human mAbs can be prepared and isolated by methods well known in the art. In some aspects, the anti-PD-L1 antibody is selected from the group consisting of BMS-936559 (also known as 12A4, MDX-1105; see, e.g., U.S. Pat. No. 7,943,743 and WO 2013/173223), atezolizumab (Roche; also known as TECENTRIQ®, MPDL3280A, RG7446; see U.S. Pat. No. 8,217,149; see, also, Herbst et al. (2013) J Clin Oncol 31 (suppl): 3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), and CK-301 (Checkpoint Therapeutics; see Gorelik et al., AACR:Abstract 4606 (April 2016)).

In some aspects, the PD-L1 antibody is atezolizumab (TECENTRIQ®). Atezolizumab is a fully humanized IgG1 monoclonal anti-PD-L1 antibody.

In some aspects, the PD-L1 antibody is durvalumab (IMFINZI™). Durvalumab is a human IgG1 kappa monoclonal anti-PD-L1 antibody.

In some aspects, the PD-L1 antibody is avelumab (BAVENCIO®). Avelumab is a human IgG1 lambda monoclonal anti-PD-L1 antibody.

In some aspects, the anti-PD-L1 monoclonal antibody is selected from the group consisting of 28-8, 28-1, 28-12, 29-8, 5H1, and any combination thereof.

In some aspects, the anti-PD-L1 antibodies of the disclosed methods comprise isolated antibodies that bind specifically to human PD-L1 and cross-compete for binding to human PD-L1 with any anti-PD-L1 antibody disclosed herein, e.g., atezolizumab, durvalumab, and/or avelumab. In some embodiments, the anti-PD-L1 antibody binds the same epitope as any of the anti-PD-L1 antibodies described herein, e.g., atezolizumab, durvalumab, and/or avelumab. Cross-competing antibodies can be readily identified based on their ability to cross-compete with atezolizumab and/or avelumab in standard PD-L1 binding assays such as Biacore analysis, ELISA assays or flow cytometry (see, e.g., WO 2013/173223).

In some aspects, the antibodies that cross-compete for binding to human PD-L1 with, or bind to the same epitope region of human PD-L1 antibody as, atezolizumab, durvalumab, and/or avelumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-L1 antibodies for use in the methods disclosed herein can include antigen-binding portions of the above antibodies. It has been amply demonstrated that the antigen-binding function of an antibody can be performed by fragments of a full-length antibody.

### CTLA-4

Monoclonal antibodies that specifically bind to CTLA-4 include, without limitation, Ipilimumab (Yervoy®; BMS) and Tremelimumab (AstraZeneca/MedImmune), as well as antibodies disclosed in U.S. Patent Application Publication Nos. 2005/0201994, 2002/0039581, and 2002/0086014, the contents of each of which are incorporated herein by reference, and antibodies disclosed in U.S. Pat. Nos. 5,811,097; 5,855,887; 6,051,227; 6,984,720; 6,682,736; 6,207,156; 5,977,318; 6,682,736; 7,109,003; 7,132,281; and 8,491,895 the contents of each of which are incorporated herein by reference, or an antibody comprising the heavy and light chain variable regions of any of these antibodies.

Human monoclonal antibodies that bind specifically to CTLA-4 with high affinity have been disclosed in U.S. Pat. Nos. 6,984,720. Other anti-CTLA-4 monoclonal antibodies have been described in, for example, U.S. Pat. No. 7,034,121 and International Publication Nos. WO 2012/122444, WO 2007/113648, WO 2016/196237, and WO 2000/037504. In some aspects, the immune checkpoint inhibitor is a CTLA-4 antagonist. In some aspects, the CTLA-4 antagonist is selected from the group consisting of ipilimumab and tremelimumab. In some aspects, the CTLA-4 antagonist is ipilimumab.

In some aspects, the anti-CTLA-4 antibodies of the disclosed methods comprise isolated antibodies that bind specifically to human CTLA-4 and cross-compete for binding to human CTLA-4 with any anti-CTLA-4 antibody disclosed herein, e.g., ipilimumab. In some embodiments, the anti-CTLA-4 antibody binds the same epitope as any of the anti-CTLA-4 antibodies described herein, e.g., ipilimumab. Cross-competing antibodies can be readily identified based on their ability to cross-compete with ipilimumab in standard CTLA-4 binding assays such as Biacore analysis, ELISA assays or flow cytometry (see, e.g., WO 2013/173223).

In some aspects, the antibodies that cross-compete for binding to human CTLA-4 with, or bind to the same epitope region of CTLA-4 as ipilimumab. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-CTLA-4 antibodies for use in the methods disclosed herein can include antigen-binding portions of the above antibodies. It has been amply demonstrated that the antigen-binding function of an antibody can be performed by fragments of a full-length antibody.

### LAG-3

The pathways involving LAG-3, BTLA, B7-H3, B7-H4, TIM-3 and KIR constitute immune checkpoint pathways similar to the CTLA-4 and PD-1 dependent pathways (see e.g., Pardoll, 2012, Nature Rev Cancer 12:252-264; Mellman et al., 2011, Nature 480:480-489).

Anti-human-LAG-3 antibodies (or VH/VL domains derived therefrom) suitable for use in the combination therapy disclosed herein can be generated using methods well known in the art. Alternatively, art recognized anti-LAG-3 antibodies can be used.

For example, the anti-human LAG-3 antibody described in US2011/0150892 A1, the teachings of which are hereby incorporated by reference, and referred to as monoclonal antibody 25F7 (also known as “25F7” and “LAG3.1”) can be used. Other art recognized anti-LAG-3 antibodies that can be used include IMP731 (H5L7BW) described in US 2011/007023, MK-4280 (28G-10) described in WO2016028672, REGN3767 described in Journal for ImmunoTherapy of Cancer, (2016) Vol. 4, Supp. Supplement 1 Abstract Number:P 195, BAP050 described in WO2017/019894, IMP-701 (LAG-525), IMP321 (eftilagimod alpha)), Sym022, TSR-033, MGD013, BI754111, FS118, AVA-017 and GSK2831781. These and other anti-LAG-3 antibodies useful in the claimed invention can be found in, for example: WO2016/028672, WO2017/106129, WO2017/062888, WO2009/044273, WO2018/069500, WO2016/126858, WO2014/179664, WO2016/200782, WO2015/200119, WO2017/019846, WO2017/198741, WO2017/220555, WO2017/220569, WO2018/071500, WO2017/015560, WO2017/025498, WO2017/087589, WO2017/087901, WO2018/083087, WO2017/149143, WO2017/219995, US2017/0260271, WO2017/086367, WO/2017/086419, WO2018/034227, and WO2014/140180. The contents of each of these references are herein incorporated by reference.

Antibodies that compete with any of the above-referenced antibodies for binding to LAG-3 also can be used.

An exemplary anti-LAG-3 antibody is BMS-986016, as described in U.S. Pat. No. 9,505,839, which is herein incorporated by reference. In some aspects, the anti-LAG-3 antibody is BMS-986016.

In some aspects, the antibody has the heavy and light chain CDRs or variable regions of BMS-986016. In some aspects, the antibody competes for binding with and/or binds to the same epitope on LAG-3 as the above-mentioned antibodies. In some aspects, the antibody binds an epitope of human LAG-3 comprising the amino acid sequence PGHPLAPG (SEQ ID NO: 82). In some aspects, the antibody binds an epitope of human LAG-3 comprising the amino acid sequence HPAAPSSW (SEQ ID NO: 83) or PAAPSSWG (SEQ ID NO: 84).

### IV. Anticancer Agents

In some aspects of the combination therapy or methods disclosed herein, the anticancer agent is a chemotherapeutic agent selected from the group consisting of taxanes, platinums, adriamycins, cylcophosphamide, topotecan, carmustine (BCNU) or a combination thereof. In some aspects, the anti-cancer therapy is selected from the group consisting of paclitaxel, carboplatin, docetaxel, nab-paclitaxel, doxorubicin and any combination thereof.

In some aspects, the chemotherapeutic agent is selected from a group consisting of topoisomerase inhibitors (e.g., irinotecan, topotecan, doxorubicin, epirubicin, idarubicin), anti-microtubule agents (e.g., paclitaxel, docetaxel), alkylating agents (e.g., cyclophosphamide, dacarbizine), platinum-based drugs (cisplatin, carboplatin, oxaliplatin), anti-metabolites (e.g., gemcitabine, methotrexate, 5-fluorouracil), or combinations thereof.

In some aspects, the anticancer agent is doxorubicin.

In some aspects, the anti-cancer agent comprises paclitaxel.

In some aspects, the anti-cancer agent comprises carboplatin.

In some aspects, the anti-cancer agent comprises docetaxel.

In some aspects, the anti-cancer agent comprises nab-paclitaxel.

In some aspects, the anti-cancer agent comprises.

In some aspects, the anticancer agent is administered prior to the interval cytoreductive surgery every three weeks for about 12 weeks to about 18 weeks.

In some aspects, the anticancer agent is administered prior to the interval cytoreductive surgery every three weeks for about 12 weeks to about 18 weeks.

In some aspects, the anticancer agent is administered at least about 28 days after the interval cytoreductive surgery, every three weeks for about 9 weeks.

In some aspects, the anticancer agent is selected from the group consisting of paclitaxel, carboplatin, docetaxel, nab-paclitaxel, and any combination thereof.

In some aspects, the nucleic acid vector formulated with the lipopolymer is administered prior to, concurrently with, or after the anticancer agent.

In some aspects, an anticancer agent is administered (e.g., first), followed by the administration of the nucleic acid vector formulated with the lipopolymer (e.g., second), and followed by administration of the immune checkpoint inhibitor. (e.g., third).

In some aspects, an anticancer agent is administered (e.g., first), followed by the administration of the nucleic acid vector formulated with the lipopolymer (e.g., second), followed by the immune checkpoint inhibitor (e.g. third), and followed by a surgery to remove all or part of a tissue or tumor (e.g., interval cytoreductive surgery) (e.g., fourth).

In some aspects, a surgery to remove all or part of a tissue or tumor (e.g., interval cytoreductive surgery) is administered (e.g., first), followed by the administration of the nucleic acid vector formulated with the lipopolymer is administered (e.g., second), and followed by administration of an immune checkpoint inhibitor (e.g., third, fourth, etc. depending on how many immune checkpoint inhibitors are administered). In some aspects, an anticancer agent is administered, followed by the administration of a DNA plasmid, followed by the immune checkpoint inhibitor, followed by an interval cytoreductive surgery.

In some aspects, the anticancer agent is administered prior to the interval cytoreductive surgery every three weeks for about 12 weeks to about 18 weeks.

In some aspects, the anticancer agent is administered at least about 28 days after the interval cytoreductive surgery (e.g., every three weeks for about 9 weeks).

In some aspects, the administration of the anticancer agent comprises administering paclitaxel at a dose of about 25-250 mg/m², optionally, followed by administering carboplatin at a dose of about AUC 4-6 IV.

In some aspects, the administration of the anticancer agent comprises administering paclitaxel at a dose of about 25-250 mg/m², about 50-250 mg/m², about 75-250 mg/m², about 100-250 mg/m², about 125-250 mg/m², about 150-250 mg/m², about 175-250 mg/m², about 200-250 mg/m², about 225-250 mg/m², about 25-225 mg/m², about 25-200 mg/m², about 25-175 mg/m², about 25-150 mg/m², about 25-125 mg/m², about 25-250 mg/m², about 25-100 mg/m², about 25-75 mg/m², or about 25-50 mg/m², optionally, followed by administering carboplatin at a dose of about AUC 4-6 IV.

In some aspects, the administration of the anticancer agent comprises administering paclitaxel at a dose of about 25 mg/m², about 50 mg/m², about 75 mg/m², about 100 mg/m², about 125 mg/m², about 150 mg/m², about 175 mg/m², about 200 mg/m², about 225 mg/m², or about 250 mg/m², optionally, followed by administering carboplatin at a dose of about AUC 4-6 IV.

In some aspects, the administration of the anticancer agent comprises administering docetaxel at a dose of 25-250 mg/m², optionally, followed by administering carboplatin at a dose of about AUC 4-6 IV.

In some aspects, the administration of the anticancer agent comprises administering docetaxel at a dose of about 25-250 mg/m², about 50-250 mg/m², about 75-250 mg/m², about 100-250 mg/m², about 125-250 mg/m², about 150-250 mg/m², about 175-250 mg/m², about 200-250 mg/m², about 225-250 mg/m², about 25-225 mg/m², about 25-200 mg/m², about 25-175 mg/m², about 25-150 mg/m², about 25-125 mg/m², about 25-250 mg/m², about 25-100 mg/m², about 25-75 mg/m², or about 25-50 mg/m², optionally, followed by administering carboplatin at a dose of about AUC 4-6 IV.

In some aspects, the administration of the anticancer agent comprises administering docetaxel at a dose of about 25 mg/m², about 50 mg/m², about 75 mg/m², about 100 mg/m², about 125 mg/m², about 150 mg/m², about 175 mg/m², about 200 mg/m², about 225 mg/m², or about 250 mg/m², optionally, followed by administering carboplatin at a dose of about AUC 4-6 IV.

In some aspects, the administration of the anticancer agent comprises administering nab-paclitaxel at a dose of 25-350 mg/m², optionally followed by administering carboplatin at a dose of about AUC 4-6 IV.

In some aspects, the administration of the anticancer agent comprises administering nab-paclitaxel at a dose of about 25-250 mg/m², about 50-250 mg/m², about 75-250 mg/m², about 100-250 mg/m², about 125-250 mg/m², about 150-250 mg/m², about 175-250 mg/m², about 200-250 mg/m², about 225-250 mg/m², about 25-225 mg/m², about 25-200 mg/m², about 25-175 mg/m², about 25-150 mg/m², about 25-125 mg/m², about 25-250 mg/m², about 25-100 mg/m², about 25-75 mg/m², or about 25-50 mg/m², optionally, followed by administering carboplatin at a dose of about AUC 4-6 IV.

In some aspects, the administration of the anticancer agent comprises administering nab-paclitaxel at a dose of about 25 mg/m², about 50 mg/m², about 75 mg/m², about 100 mg/m², about 125 mg/m², about 150 mg/m², about 175 mg/m², about 200 mg/m², about 225 mg/m², or about 250 mg/m², optionally, followed by administering carboplatin at a dose of about AUC 4-6 IV.

In some aspects, the administration of the nanoparticle prior to the interval cytoreductive surgery begins 15 days after the first administration of the anticancer agent, every week for at least about 12 weeks to about 18 weeks.

In some aspects, the administration of the anticancer agent comprises administering paclitaxel at a dose of about 175 mg/m², followed by administering carboplatin at a dose of about AUC 4-6 IV.

In some aspects, the administration of the anticancer agent comprises administering docetaxel at a dose of about 75 mg/m², followed by administering carboplatin at a dose of about AUC 4-6 IV.

In some aspects, the administration of the anticancer agent comprises administering nab-paclitaxel at a dose of about 260 mg/m², followed by administering carboplatin at a dose of about AUC 4-6 IV.

### V. Methods of Treatment

Certain aspects of the disclosure are related to a method of treating a subject suffering from cancer comprising administering to the subject a combination of (i) a nanoparticle comprising an IL-2 coding sequence (e.g., a nucleic acid is a plasmid-based gene expression system containing a DNA sequence which encodes an interleukin-12), and (ii) an immune checkpoint inhibitor. In some aspect, the method further comprises administering a chemotherapeutic agent to the subject. In some aspects, the method further comprises a surgery. In some aspects, the nanoparticle comprises a PEG-PEI-cholesterol (PPC) lipopolymer.

This present disclosure also provides a method for treatment of mammalian cancer or hyperproliferative disorders by intratumoral, intraperitoneal, intravenously, intravesicularly, intratracheal, intracranial or systemic administration of pharmaceutical compositions comprising a plasmid-based gene expression system and a gene delivery polymer, without a chemotherapeutic drug. The mammalian cancer is selected from a group consisting of primary or metastasized tumors of the ovary. In some aspects, the nucleic acid is a plasmid-based gene expression system containing a DNA sequence which encodes human interleukin-12.

The treatment of tumors with the said pharmaceutical composition (nucleic acid plus gene delivery polymer and one or more chemotherapeutic agents) results in tumor shrinkage and extension of life span. The combination of gene therapy (nucleic acid and gene delivery polymers) with chemotherapy (chemotherapeutic agents) according to the method of the present disclosure produce additive and/or synergistic efficacy. The efficacy of the method of this invention is defined as but not limited to shrinkage in tumor size or reduction in tumor density, an increase in lymphocyte count or increase in neutrophil count or improvement in survival, or all of the above. In addition, the combination of gene therapy (nucleic acid and gene delivery polymers) with chemotherapy (chemotherapeutic agents) according to the method of the present invention lowers the toxicity of the chemotherapeutic agent and reverses tumor resistance to chemotherapy. The toxicity herein is defined as any treatment related adverse effects on clinical observation including but not limited to abnormal hematology or serum chemistry or organ toxicity. Furthermore, the combination of gene therapy (nucleic acid and gene delivery polymers) with a suboptimal dose of chemotherapy (chemotherapeutic agents) according to the method of the present invention enhances the anticancer effect to a level equal to or higher than that of achieved with the optimal dose of the chemotherapeutic agent but with lesser toxicity.

New cancer treatment strategies are focused on delivering macromolecules carrying genetic information, rather than a therapeutic protein itself, allowing for the exogenously delivered genes to be expressed in the tumor environment. Methods that utilize non-viral gene delivery systems are considered safer compared to viral delivery systems, but the practical application of current polymeric systems has not been satisfactory due to poor efficiency. A strategy has recently been disclosed whereby the gene transfection efficiency of a low molecular weight PEI was enhanced by covalent attachment of cholesterol forming a water soluble lipopolymer (WSLP). See, Mol. Ther., 2001, 4, 130. IL-12 gene transfer to solid tumors with WSLP was significantly better than by the unmodified PEI and led to more significant tumor inhibition.

It is recognized that a single treatment strategy against cancer is generally ineffective due to the multi-factorial nature of this disease. The methods disclosed herein provide a benefit from the combination of more than one drug or therapeutic to maximize anticancer response. In some aspects, combined here is a chemotherapeutic agent and an immune checkpoint inhibitor with gene delivery of an anticancer IL-12 expression construct (e.g., GEN-1). Combining safe and efficient delivery of an anticancer gene with a standard chemotherapeutic agent and an immune checkpoint inhibitor can enhance anticancer response. This combination therapy can be used to reduce the chemotherapy dose and increase tumor sensitivity to the chemotherapy. In some aspects, the compositions disclosed herein can comprise an anticancer gene complexed with a gene delivery polymer, an immune checkpoint inhibitor, and at least one adjunctive chemotherapeutic drug, wherein the combination is more effective than gene therapy, immune checkpoint inhibitor, or chemotherapy treatment administered alone. Furthermore, the combination therapy disclosed herein can be effective against a wide variety of tumors and given by different routes of administration.

CTLA4 blockade can significantly increase CD8 T cell infiltration of the tumor microenvironment (TME). CTLA4 and PD1 blockade mechanisms are non-redundant leading to enhanced immune and clinical responses in combination. However, the majority of patients with solid tumors do not respond to these agents where the TME is considered less immunogenic where proinflammatory cytokines such as IL-12 may play an important role in transforming the TME working synergistically with CTLA4 blockade and PD1 blockade. IL-12 has been shown to enhance the homeostatic proliferation of CD8+ T cells in vivo, and CD8+ T cells were significantly increased in biopsy specimens obtained from cancer patients treated with IL-12.

IL-12 is a potent pro-inflammatory cytokine that promotes CD4+ cell differentiation into Th1 cells and has synergistic proliferative effects on pre-activated NK and T cells. IL-12 also enhances independently and/or synergistically the cytolytic ability of both NK and CD8+ T cells. Antitumor activity of systemic or local administration of IL-12 has been established in preclinical studies against various tumor cell lines. However, the potent pro-inflammatory antitumor immune response mediated by IL-12 may still be countered through tumor mediated immune suppressive mechanisms where PD-1: PD-L interaction plays a key role. IL-12 induction of IFN-γ and other proinflammatory cytokines is well documented. IFNγ is a pleotropic cytokine that has been reported to promote tumor immunity and tumor rejection in many models. In these models, IFNγ has anti-proliferative and pro-apoptotic functions and inhibits tumor angiogenesis. Several studies have shown the importance of IFNγ in the efficacy of tumor vaccines and IL-12 therapies. In clinical trials, IFNγ therapy has been associated with melanoma regression in some patients. Nevertheless, clinical trials in patients with melanoma using IFNγ alone or in combination with IFNα failed to show improved survival compared with other therapies. Consistent with these findings, several studies have demonstrated that IFNγ stimulation can induce up-regulation of PD-L1 in monocytes, tumor cells and other cell types. In one study, expression of PD-L1 was increased in every cell line and primary cell incubated with IFNy.

Certain aspects of the disclosure are related to a combination therapy comprising: a (i) a nucleic acid vector (e.g., a plasmid) comprising a polynucleotide that encodes an interleukin-12 (IL-12) formulated with a lipopolymer (e.g., a nanoparticle); and (ii) an immune checkpoint inhibitor (e.g., an antibody).

Certain aspects of the disclosure are related to a method of treating a subject suffering from cancer comprising administering to the subject a combination therapy comprising: (i) a nucleic acid vector (e.g., a plasmid) comprising a polynucleotide that encodes an interleukin-12 (IL-12) formulated with a lipopolymer (e.g., a nanoparticle); and (ii) an immune checkpoint inhibitor (e.g., an antibody).

In some aspects, the polynucleotide encodes human IL-12.

In some aspects, nucleic acid vector (e.g., a plasmid) comprises a promoter operably linked to a nucleic acid encoding a p35 subunit of IL-12 and a promoter operably linked to a nucleic acid encoding a p40 subunit of IL12.

In some aspects, nucleic acid vector (e.g., a plasmid) comprises an intron, a 3′ UTR (e.g., hGH 3′ UTR), an antibiotic resistance gene, or any combination thereof (e.g., the elements of FIG. 1).

In some aspects, the lipopolymer comprises of polyethyleneimine (PEI) covalently linked independently to cholesterol and polyethylene glycol (PEG) groups (e.g., the lipopolymer of FIG. 2).

In some aspects, the immune checkpoint inhibitor is an inhibitor of an immune checkpoint protein selected from the group consisting of CTLA-4, PD-1 (and its ligands PD-L1 and PD-L2), and/or LAG-3.

In some aspects, the immune checkpoint inhibitor is an antibody.

In some aspects, the immune checkpoint inhibitor is a small molecule inhibitor.

In some aspects, the immune checkpoint inhibitor is a PD-1 antagonist selected from the group consisting of nivolumab, pembrolizumab, dostarlimab, and cemiplimab.

In some aspects, the immune checkpoint inhibitor is a PD-1 antagonist, wherein the PD-1 antagonist is nivolumab.

In some aspects, the immune checkpoint inhibitor is a PD-L1 antagonist selected from the group consisting of atezolizumab, durvalumab, and avelumab.

In some aspects, the immune checkpoint inhibitor is a CTLA-4 antagonist is ipilimumab.

In some aspects, the immune checkpoint inhibitor is a LAG-3 antagonist, wherein the LAG-3 antagonist is relatlimab.

In some aspects, the combination further comprises an anticancer agent.

In some aspects, the anticancer agent is a chemotherapeutic agent.

In some aspects, the chemotherapeutic agent is selected from the group consisting of doxorubicin, paclitaxel, carboplatin, docetaxel, nab-paclitaxel, olaparib, and any combination thereof.

In some aspects, the anticancer agent is doxorubicin.

In some aspects, the anticancer agent is paclitaxel.

In some aspects, the anticancer agent is carboplatin.

In some aspects, the anticancer agent is docetaxel.

In some aspects, the anticancer agent is nab-paclitaxel.

In some aspects, the anticancer agent is olaparib.

In some aspect, the method further comprises a surgery to remove all or part of a tissue or tumor (e.g., interval cytoreductive surgery) in the subject.

In some aspects, the nucleic acid vector formulated with the lipopolymer is administered intratumorally or intraperitoneally.

In some aspects, the nucleic acid vector formulated with the lipopolymer is administered intravenously.

In some aspects, the immune checkpoint inhibitor is administered intratumorally intraperitoneally, intravesicularly, or any combination thereof.

In some aspects, the immune checkpoint inhibitor is administered intratumorally or intraperitoneally.

In some aspects, the immune checkpoint inhibitor is administered intravenously.

In some aspects, the nucleic acid vector formulated with the lipopolymer is administered prior to, concurrently with, or after the immune checkpoint inhibitor.

In some aspects, the nucleic acid vector formulated with the lipopolymer is administered prior to, concurrently with, or after the anticancer agent.

In some aspects, an anticancer agent is administered (e.g., first), followed by the administration of the nucleic acid vector formulated with the lipopolymer (e.g., second), and followed by administration of the immune checkpoint inhibitor (e.g., third).

In some aspects, an anticancer agent is administered (e.g., first), followed by the administration of the nucleic acid vector formulated with the lipopolymer (e.g., second), followed by the immune checkpoint inhibitor (e.g. third), and followed by a surgery to remove all or part of a tissue or tumor (e.g., interval cytoreductive surgery) (e.g., fourth).

In some aspects, a surgery to remove all or part of a tissue or tumor (e.g., interval cytoreductive surgery) is administered (e.g., first), followed by the administration of the nucleic acid vector formulated with the lipopolymer is administered (e.g., second), and followed by administration of an immune checkpoint inhibitor (e.g., third, fourth, etc. depending on how many immune checkpoint inhibitors are administered).

In some aspects, an anticancer agent is administered, followed by the administration of a DNA plasmid, followed by the immune checkpoint inhibitor, followed by an interval cytoreductive surgery.

In some aspects, the anticancer agent is administered prior to the interval cytoreductive surgery every three weeks for about 12 weeks to about 18 weeks.

In some aspects, the anticancer agent is administered at least about 28 days after the interval cytoreductive surgery (e.g., every three weeks for about 9 weeks).

In some aspects, the administration of the anticancer agent comprises administering paclitaxel at a dose of about 25-250 mg/m², optionally, followed by administering carboplatin at a dose of about AUC 4-6 IV.

In some aspects, the administration of the anticancer agent comprises administering paclitaxel at a dose of about 25-250 mg/m², about 50-250 mg/m², about 75-250 mg/m², about 100-250 mg/m², about 125-250 mg/m², about 150-250 mg/m², about 175-250 mg/m², about 200-250 mg/m², about 225-250 mg/m², about 25-225 mg/m², about 25-200 mg/m², about 25-175 mg/m², about 25-150 mg/m², about 25-125 mg/m², about 25-250 mg/m², about 25-100 mg/m², about 25-75 mg/m², or about 25-50 mg/m², optionally, followed by administering carboplatin at a dose of about AUC 4-6 IV.

In some aspects, the administration of the anticancer agent comprises administering paclitaxel at a dose of about 25 mg/m², about 50 mg/m², about 75 mg/m², about 100 mg/m², about 125 mg/m², about 150 mg/m², about 175 mg/m², about 200 mg/m², about 225 mg/m², or about 250 mg/m², optionally, followed by administering carboplatin at a dose of about AUC 4-6 IV.

In some aspects, the administration of the anticancer agent comprises administering docetaxel at a dose of 25-250 mg/m², optionally, followed by administering carboplatin at a dose of about AUC 4-6 IV.

In some aspects, the administration of the anticancer agent comprises administering docetaxel at a dose of about 25-250 mg/m², about 50-250 mg/m², about 75-250 mg/m², about 100-250 mg/m², about 125-250 mg/m², about 150-250 mg/m², about 175-250 mg/m², about 200-250 mg/m², about 225-250 mg/m², about 25-225 mg/m², about 25-200 mg/m², about 25-175 mg/m², about 25-150 mg/m², about 25-125 mg/m², about 25-250 mg/m², about 25-100 mg/m², about 25-75 mg/m², or about 25-50 mg/m², optionally, followed by administering carboplatin at a dose of about AUC 4-6 IV.

In some aspects, the administration of the anticancer agent comprises administering docetaxel at a dose of about 25 mg/m², about 50 mg/m², about 75 mg/m², about 100 mg/m², about 125 mg/m², about 150 mg/m², about 175 mg/m², about 200 mg/m², about 225 mg/m², or about 250 mg/m², optionally, followed by administering carboplatin at a dose of about AUC 4-6 IV.

In some aspects, the administration of the anticancer agent comprises administering nab-paclitaxel at a dose of 25-350 mg/m², optionally followed by administering carboplatin at a dose of about AUC 4-6 IV.

In some aspects, the administration of the anticancer agent comprises administering nab-paclitaxel at a dose of about 25-250 mg/m², about 50-250 mg/m², about 75-250 mg/m², about 100-250 mg/m², about 125-250 mg/m², about 150-250 mg/m², about 175-250 mg/m², about 200-250 mg/m², about 225-250 mg/m², about 25-225 mg/m², about 25-200 mg/m², about 25-175 mg/m², about 25-150 mg/m², about 25-125 mg/m², about 25-250 mg/m², about 25-100 mg/m², about 25-75 mg/m², or about 25-50 mg/m², optionally, followed by administering carboplatin at a dose of about AUC 4-6 IV.

In some aspects, the administration of the anticancer agent comprises administering nab-paclitaxel at a dose of about 25 mg/m², about 50 mg/m², about 75 mg/m², about 100 mg/m², about 125 mg/m², about 150 mg/m², about 175 mg/m², about 200 mg/m², about 225 mg/m², or about 250 mg/m², optionally, followed by administering carboplatin at a dose of about AUC 4-6 IV.

In some aspects, the administration of the nanoparticle prior to the interval cytoreductive surgery begins 15 days after the first administration of the anticancer agent, every week for at least about 12 weeks to about 18 weeks.

In some aspects, the nanoparticle is administered at least about 28 days following the interval cytoreductive surgery, and administration begins 15 days after the first administration of the anticancer agent, every week for at least about 9 weeks.

In some aspects, the interleukin-12 (IL-12) formulated with a lipopolymer (e.g., a nanoparticle) is administered at a dose of about 35 mg/m² to about 80 mg/m².

In some aspects, the nanoparticle is administered at a dose of about 35 mg/m² to about 80 mg/m². In some aspects, the nanoparticle is administered at a dose of about 40 mg/m² to about 80 mg/m². In some aspects, the nanoparticle is administered at a dose of about 45 mg/m² to about 80 mg/m². In some aspects, the nanoparticle is administered at a dose of about 50 mg/m² to about 80 mg/m². In some aspects, the nanoparticle is administered at a dose of about 55 mg/m² to about 80 mg/m². In some aspects, the nanoparticle is administered at a dose of about 60 mg/m² to about 80 mg/m². In some aspects, the nanoparticle is administered at a dose of about 65 mg/m² to about 80 mg/m². In some aspects, the nanoparticle is administered at a dose of about 70 mg/m² to about 80 mg/m². In some aspects, the nanoparticle is administered at a dose of about 75 mg/m² to about 80 mg/m². In some aspects, the nanoparticle is administered at a dose of about 35 mg/m² to about 75 mg/m². In some aspects, the nanoparticle is administered at a dose of about 35 mg/m² to about 70 mg/m². In some aspects, the nanoparticle is administered at a dose of about 35 mg/m² to about 65 mg/m². In some aspects, the nanoparticle is administered at a dose of about 35 mg/m² to about 60 mg/m². In some aspects, the nanoparticle is administered at a dose of about 35 mg/m² to about 55 mg/m². In some aspects, the nanoparticle is administered at a dose of about 35 mg/m² to about 50 mg/m². In some aspects, the nanoparticle is administered at a dose of about 35 mg/m² to about 45 mg/m². In some aspects, the nanoparticle is administered at a dose of about 35 mg/m² to about 40 mg/m².

In some aspects, the nanoparticle is administered at a dose of about 35 mg/m², about 40 mg/m², about 45 mg/m², about 50 mg/m², about 55 mg/m², about 60 mg/m², about 65 mg/m², about 70 mg/m², about 75 mg/m², or about 80 mg/m².

In some aspects, the nanoparticle is administered at a dose of about 60 mg/m².

In some aspects, the immune checkpoint inhibitor is ipilimumab. In some aspects, ipilimumab is administered at a dose of about 0.5 to about 5 mg/kg. In some aspects, ipilimumab is administered at a dose of about 1 to about 5 mg/kg. In some aspects, ipilimumab is administered at a dose of about 1.5 to about 5 mg/kg. In some aspects, ipilimumab is administered at a dose of about 2 to about 5 mg/kg. In some aspects, ipilimumab is administered at a dose of about 2.5 to about 5 mg/kg. In some aspects, ipilimumab is administered at a dose of about 3 to about 5 mg/kg. In some aspects, ipilimumab is administered at a dose of about 3.5 to about 5 mg/kg. In some aspects, ipilimumab is administered at a dose of about 4 to about 5 mg/kg. In some aspects, ipilimumab is administered at a dose of about 0.5 to about 4 mg/kg. In some aspects, ipilimumab is administered at a dose of about 0.5 to about 3 mg/kg. In some aspects, ipilimumab is administered at a dose of about 0.5 to about 2.5 mg/kg. In some aspects, ipilimumab is administered at a dose of about 0.5 to about 2 mg/kg. In some aspects, ipilimumab is administered at a dose of about 0.5 to about 1.5 mg/kg.

In some aspects, ipilimumab is administered at a dose of about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, or about 5 mg/kg.

In some aspects, ipilimumab is administered at a dose of about 1 mg/kg.

In some aspects, ipilimumab is administered every 1 week, 2 weeks, 3 weeks, 4 weeks, 5 weeks, 6 weeks, 7 weeks, or 8 weeks.

In some aspects, ipilimumab is administered every 2-8 weeks (e.g., every 6 weeks) during the treatment.

In some aspects, ipilimumab is administered intravenously.

In some aspects, the immune checkpoint inhibitor is nivolumab. In some aspects, nivolumab is administered at a dose of about 120 mg to about 360 mg. In some aspects, nivolumab is administered at a dose of about 140 mg to about 360 mg. In some aspects, nivolumab is administered at a dose of about 160 mg to about 360 mg. In some aspects, nivolumab is administered at a dose of about 180 mg to about 360 mg. In some aspects, nivolumab is administered at a dose of about 200 mg to about 360 mg. In some aspects, nivolumab is administered at a dose of about 220 mg to about 360 mg. In some aspects, nivolumab is administered at a dose of about 240 mg to about 360 mg. In some aspects, nivolumab is administered at a dose of about 260 mg to about 360 mg. In some aspects, nivolumab is administered at a dose of about 280 mg to about 360 mg. In some aspects, nivolumab is administered at a dose of about 300 mg to about 360 mg. In some aspects, nivolumab is administered at a dose of about 320 mg to about 360 mg. In some aspects, nivolumab is administered at a dose of about 340 mg to about 360 mg. In some aspects, nivolumab is administered at a dose of about 120 mg to about 340 mg. In some aspects, nivolumab is administered at a dose of about 120 mg to about 320 mg. In some aspects, nivolumab is administered at a dose of about 120 mg to about 300 mg. In some aspects, nivolumab is administered at a dose of about 120 mg to about 280 mg. In some aspects, nivolumab is administered at a dose of about 120 mg to about 260 mg. In some aspects, nivolumab is administered at a dose of about 120 mg to about 240 mg. In some aspects, nivolumab is administered at a dose of about 120 mg to about 220 mg. In some aspects, nivolumab is administered at a dose of about 120 mg to about 200 mg. In some aspects, nivolumab is administered at a dose of about 120 mg to about 180 mg. In some aspects, nivolumab is administered at a dose of about 120 mg to about 160 mg. In some aspects, nivolumab is administered at a dose of about 120 mg to about 140 mg. In some aspects, nivolumab is administered at a dose of about 240 mg to about 360 mg. In some aspects, nivolumab is administered at a dose of about 260 mg to about 360 mg. In some aspects, nivolumab is administered at a dose of about 280 mg to about 360 mg. In some aspects, nivolumab is administered at a dose of about 300 mg to about 360 mg. In some aspects, nivolumab is administered at a dose of about 320 mg to about 360 mg. In some aspects, nivolumab is administered at a dose of about 340 mg to about 360 mg. In some aspects, nivolumab is administered at a dose of about 140 mg to about 240 mg. In some aspects, nivolumab is administered at a dose of about 160 mg to about 240 mg. In some aspects, nivolumab is administered at a dose of about 180 mg to about 240 mg. In some aspects, nivolumab is administered at a dose of about 200 mg to about 240 mg. In some aspects, nivolumab is administered at a dose of about 220 mg to about 240 mg.

In some aspects, nivolumab is administered at a dose of about 120 mg, about 140 mg, about 160 mg, about 180 mg, about 200 mg, about 220 mg, about 240 mg, about 260 mg, about 280 mg, about 300 mg, about 320 mg, about 340 mg, or about 360 mg.

In some aspects, nivolumab is administered at a dose of about 240 mg.

In some aspects, nivolumab is administered intravenously.

In some aspects, nivolumab is administered every 1 week, 2 weeks, 3 weeks, 4 weeks, 5 weeks, 6 weeks, 7 weeks, or 8 weeks.

In some aspects, nivolumab is administered every 1-4 weeks (e.g., every 2 weeks) during treatment.

In some aspects, the method further comprises administering a second immune checkpoint inhibitor.

In some aspects, the second immune checkpoint inhibitor is an inhibitor of an immune checkpoint protein selected from the group consisting of CTLA-4, PD-1 (and its ligands PD-L1 and PD-L2), and/or LAG-3.

In some aspects, the second immune checkpoint inhibitor is an antibody.

In some aspects, the second immune checkpoint inhibitor is a small molecule inhibitor.

In some aspects, the second immune checkpoint inhibitor is a PD-1 antagonist selected from the group consisting of nivolumab, pembrolizumab, dostarlimab, and cemiplimab.

In some aspects, the second immune checkpoint inhibitor is a PD-1 antagonist, wherein the PD-1 antagonist is nivolumab.

In some aspects, the second immune checkpoint inhibitor is a PD-L1 antagonist selected from the group consisting of atezolizumab, durvalumab, and avelumab.

In some aspects, the second immune checkpoint inhibitor is a CTLA-4 antagonist is ipilimumab.

In some aspects, the second immune checkpoint inhibitor is a LAG-3 antagonist, wherein the LAG-3 antagonist is relatlimab.

In some aspects, the first immune checkpoint inhibitor is nivolumab, and the second immune checkpoint inhibitor is ipilimumab.

In some aspects, the nivolumab is administered at about 240 mg every 2 weeks, and the ipilimumab is administered at about 1 mg/kg every 6 weeks.

In some aspects, the immune checkpoint inhibitor is administered prior to the interval cytoreductive surgery, at least about 22 days after the first administration of the anticancer agent, every week for at least about 12 weeks to up about 18 weeks.

In some aspects, the immune checkpoint inhibitor is administered at least about 28 days after the interval cytoreductive surgery, and at least about 22 days after the first administration of the anticancer agent, every week for at least about 9 weeks.

In some aspects, the interval cytoreductive surgery (ICS) is administered at least about 28 days following the administration of an anticancer agent.

In some aspects, the interval cytoreductive surgery (ICS) is administered at least about 7 days following the administration of the DNA plasmid.

In some aspects, interval cytoreductive surgery (ICS) is administered at least about 7 days following the administration of the DNA plasmid.

In some aspects, the interval cytoreductive surgery (ICS) is administered at least about 28 days before the administration of the immune checkpoint inhibitor.

In some aspects, the interval cytoreductive surgery (ICS) is administered at least about 28 days following the administration of the immune checkpoint inhibitor.

In some aspects, the cancer is selected from a group consisting of ovarian cancer, fallopian tube cancer, primary peritoneal cancer, cervical cancer, breast cancer, prostate cancer, colorectal cancer, bladder cancer, brain cancer (e.g., glioblastoma), lung cancer, and any combination thereof, and metastasis of any of the cancers.

In some aspects, the cancer is selected from a group consisting of ovarian cancer, fallopian tube cancer, primary peritoneal cancer, and any combination thereof.

In some aspects, the subject is a human.

In some aspects, the cancer is ovarian cancer.

Ovarian cancer (OC) is the most lethal gynecologic cancer in the US. The five-year overall survival rate is 20-30% in advanced stage OC, with more than 50% of patients that respond to current therapies experiencing recurrence of their disease. Recurrent, platinum-resistant OC is characterized by only minimal responses to chemotherapy (<10-15%) and a poor prognosis, with overall survival estimated to be <12 months. Thus, there is a need to improve the outcomes of these patients. The impact of immune checkpoint inhibitors (ICI) has been significant, providing durable response rates in several cancers. However, the response rates in ovarian cancer are low, ranging from 11-15% in platinum-resistant, recurrent settings. Compared to nivolumab alone, dual immunotherapy with nivolumab and ipilimumab was associated with a higher objective response rate (31% vs. 12%) but with limited durability (3.9 vs. 2 months).

The following examples are illustrative and do not limit the scope of the claimed aspects.

### Examples

### Example 1. GEN-1 Combo Therapy with anti-CTLA-4 in murine mouse model of Ovarian Cancer

The ability of GEN-1 to synergize with dual-ICI (anti-PD1+anti-CTLA4) in the preclinical ID8-VEGF immunocompetent murine model of ovarian cancer (OC) was tested. Our results showed that a combination of IL 12 and dual-ICI resulted in enhanced durable anti-tumor activity and survival of mice bearing ID8-VEGF tumors as compared to those treated with monotherapy (results not shown). Improved anti-tumor activity was associated with increased tumor infiltration by T cells and a downregulation of the phenotypic traits of myeloid cells known to mediate immunosuppression, leading to enhanced effector function of the infiltrating T cells. These results provide preclinical data supporting the use of IL 12 as a strategy when combined with dual anti-PD1+anti-CTLA4 therapy in the treatment of OC.

### Example 2. GEN-1 Combo Therapy with Immune Checkpoint Inhibitors (Prophetic)

A combination strategy including IL-12 gene therapy (GEN-1) and CTLA4-PD-1 blockade will be tested. The mechanisms of action of known sites for activity for IL-12 and CTLA4-PD-1 blockade are non-redundant. Therefore, combination therapy of rhIL-12 and immune checkpoint inhibitors (ipilimumab and nivolumab) will be tested for anti-tumor activity. Further, the study will assess enhanced efficacy of current PD1/PD-L1-based immunotherapy by targeting pathways that mediate resistance to ICI.

### Dose Levels Of IL-12 Proposed And The Potential Of IL-12 To Enhance The Biological And Clinical Activity Of The Ipilimumab-Nivolumab (Ipi-Nivo) Combination

IL-12 has been shown to enhance the homeostatic proliferation of CD8+ T cells in vivo. CD8+ T cells were increased in biopsy specimens obtained from regressing lesions of cancer patients treated with IL-12. Correlative assays in phase I studies of IL-12 showed sustained production of IFN-γ by immune cells of treated patients. IFN-γ, a primary inducer of PD-L1 expression, was detected at the interface of PD-L1 (+) tumors and TILs, whereas none was found in PD-L1 (−) tumors. PD-L1 expression has been associated with a higher likelihood of response to anti-PD1 monoclonal antibodies and therefore, this effect of IL-12 of upregulating TIL in tumor and enhancing IFN-γ secretion will be tested for increased PD-L1 expression and improved response.

Prior studies using rhIL-12 have demonstrated the biological and antitumor activity of IL-12. This study dose-escalated the IL-12 component within cohorts of three patients a 30, 100, 300, or 500 ng/kg. Evaluation of dose-limiting toxicity and biologic endpoints suggested that the 300 ng/kg dose was both the maximally tolerated dose and the optimal biologic dose of IL-12.

While traditional chemotherapy regimens are designed to inhibit tumor growth by cytotoxic mechanisms, immunocytokine therapies are designed to elicit tumor killing by enhancing the immune system against cancer cells. Interleukin-12 (IL-12) is one of the more active immunocytokines in the induction of anticancer immunity. It is associated with immunomodulatory properties such as T-lymphocyte and natural killer (NK) cell proliferation, activation of cytotoxic T-lymphocytes (i.e., CD8+ lymphocytes), secretion of IFN-gamma, and inhibition of immunosuppressive regulatory T-cells. IL-12 also inhibits the process of tumor angiogenesis, which leads to tumor death from starvation. These multiple and pluripotent anticancer properties make IL-12 a potentially important immunotherapy.

The GEN-1 nanoparticle comprises a DNA plasmid encoding IL-12 gene and a synthetic polymer facilitating plasmid delivery. GEN-1 is designed to be delivered locally (e.g. intraperitoneally), offering the potential for cytokines to be expressed specifically in the tumor micro-environment with the goal of achieving increased efficacy while minimizing potential systemic toxicity. GEN-1 has been studied in subjects with recurrent ovarian cancer as a single agent or combination agent with standard chemotherapy. Additionally, a Phase I clinical trial in newly diagnosed ovarian cancer subjects has been completed where GEN-1 was evaluated as a combination agent with neoadjuvant chemotherapy.

The IL-12 produced by GEN-1 will be tested to assess whether CTLA4 and/or PD1/PDL1 blocking monoclonal antibodies can overcome the immunosuppressive features of the peritoneal tumors by inducing an inflamed tumor microenvironment thus enhancing the efficacy of these checkpoint inhibitors.

### Objectives

Phase I: To investigate the safety, tolerability (MTD, DLT) and phase 2 recommended dose (P2RD) of a combined regimen of GEN-1 with anti-PD1/CTLA4 (dual ICI) in subjects with recurrent or persistent ovarian tumors.

Phase II: To determine the overall objective tumor response rate for the proposed combination regimen.

### Measurement of the Primary Endpoint

Phase I: The safety, tolerability, and recommended phase 2 dose of the regimen will be evaluated by a Data and Safety Monitoring Board (DSMB). Details regarding their responsibilities will be elucidated in a DSMB charter which will be drafted for approval by the DSMB prior to initiation of the study.

Phase II: The overall objective response rate will be determined by using RECIST 1.1

In addition, the following will be assessed: the progression free survival (PFS), overall survival (OS), objective tumor response rate (ORR), overall toxicity and clinical benefit rate.

PFS will be measured by time from enrollment to the first date on which one of the following occurs: i) Death by any cause; or ii) Unequivocal progression of target lesions, non-target lesions and/or new lesions by Response Evaluation Criteria in Solid Tumors (RECIST).

OS will be measured by time from enrollment to the date of death

ORR will be measured according to Response Evaluation Criteria for Solid tumors guidelines version 1.1 (RECIST). This protocol allows for participation of subjects with both measurable and non-measurable disease, and as such, will deviate slightly from the standard RECIST as described later in the protocol.

### Design Overview

This is a single arm, multi-center phase I/II trial designed to evaluate the safety, dosing, preliminary efficacy of adding GEN-1 to fixed doses of two ICIs in patients with recurrent or persistent ovarian cancer. The dual ICI regimen will be the anti-PD1 nivolumab and anti-CTLA4 ipilimumab. Nivolumab (240 mg IV) will be administered every 2 weeks with GEN-1 (starting dose 60 mg/m²). Ipilimumab will be administered 1 mg/kg IV every 6 weeks. Dosing will continue until disease progression, unacceptable toxicity, or up to 2 years in patients without disease progression.

Maximum Tolerated Dose (MTD): A standard 3+3 design will be employed to determine the MTD of GEN-1 for this regimen. The Gen-1 starting dose of 60 mg/m² will be combined with the fixed doses of the two ICI's. If more than 1 patient out of 6 are determined to have a dose limiting toxicity (DLT) than the DSMB may recommend decreasing the dose level of GEN-1 to 45 mg/m² for evaluation. The study may proceed to a higher dose level only if 0 of 3 patients or <1 of 6 demonstrate no evidence DLT. The dose escalation schedule is presented in Table 1. If more than 1 of 6 patients demonstrate a DLT at a dose level than the previous dose level will be declared the MTD. At least 6 patients must be confirmed at the MTD before proceeding to phase 2.

The Phase II portion of the study is a two-stage design to evaluate if the treatment will yield a objective response rate (ORR) of 45% or higher. Twenty (20) patients will be enrolled in stage 1 and evaluate whether six (6) or more subjects achieve a CR or PR. If not, the study will be stopped for futility. If the stage 1 ORR target rate is met then twenty (20) additional subjects will be enrolled to test whether at least 14 of 40 subjects achieve CR or PR to confirm the confirm the response rate in this phase 2 design.

All subjects will be followed for safety, disease progression, and clinical benefit rate (objective response and durable stable disease at 12 weeks) and survival.

### Subject Selection Criteria

The following eligibility criteria are designed to select subjects who are considered appropriate for study participation. All relevant medical and non-medical conditions should be taken into consideration when deciding whether this protocol is suitable for a particular subject. Eligibility criteria may not be waived by the Investigator and are subject to review in the case of a cGCP or regulatory authority audit.

### Study Treatment And Dosing

### Determination of MTD and Phase 2 Recommended Dose.

A 3+3 dose escalation will be employed. All subjects will be monitored for safety from time of signing informed consent. Safety data from subjects who complete 2 cycles of protocol treatment is required to be eligible for dose escalation review. In general, dose escalation may occur at 0/3 DLTs, dose-reduction if >1/3 DLT, and expansion to 6 if 1/3 DLTs. The highest dose level at which <2/6 DLTs are observed will be declared the MTD. Data will be reviewed by the study DSMB and recommend dosing modifications and the phase 2 recommended dose. During the phase I portion the dual ICI's will be given at a fixed dose at all dose levels and Gen-1 dose will be escalated according to the table presented below.

TABLE 4
Dose escalation plan for phase I portion
Dose Escalation Schedule (Phase I)

| | Dose | | |
|---|---|---|---|
| | Nivolumab (mg) | Ipilimumab | Gen-1 (mg/m²) |
| Dose Level | Q2W | (mg/kg) Q6W | Q2W |
| Level 1 (Starting | 240 | 1 | 60 |
| dose) | | | |
| Level 2 | 240 | 1 | 80 |
| Level 3 | 240 | 1 | 100 |
| Level −1 | 240 | 1 | 45 |

Dual ICI (anti-PD1 and anti-CTLA4) and GEN-1

Nivolumab 240 mg IV will be administered with GEN-1 at (at DSMB recommended dose) every 2 weeks until disease progression, unacceptable toxicity, or up to 2 years in patients without disease progression.

Ipilimumab 1 mg/kg IV will be administered every 6 weeks until disease progression, unacceptable toxicity, or up to 2 years in patients without disease progression

GEN-1 (Investigational Medicinal Product)

### Formulation

Human IL-12 plasmid (phIL-12-005) is formulated with lipopolymer PEG-PEI-Cholesterol (PPC) in 10% lactose.

### Human IL-12 Plasmid

The phIL-12-005 plasmid contains hIL-12 gene expression cassette in a plasmid containing a Kanr gene. The hIL-12 gene expression cassette of phIL-12-005 contains immediate early enhancer and promoter derived from cytomegalovirus (CMV), 5′ untranslated region (UTR), synthetic intron, p35 gene, human growth hormone (hGH) 3′ UTR and polyadenylation signal sequence, CMV promoter, 5′ UTR, synthetic intron, p40 gene, hGH 3′ UTR and polyadenylation signal sequence. The two hIL-12 subunits are individually under the control of two separate CMV promoters. See FIG. 1.

### PEG-PEI-Cholesterol

PPC is composed of a PEI backbone to which polyethyleneglycol and cholesterol are independently attached via covalent linkages. The molecular weight of PEI, PEG and cholesterolcarbonyl is 1800, 550 and 414, respectively. See FIG. 2.

### Route of Administration & Administration of GEN-1

Infusion into abdominal cavity via an IP catheter.

25 mL of 0.9% Normal Saline is flushed through the IP port to check for catheter patency. Heparin should not be used to flush the catheter at the time of sample collection or drug infusion.

Reconstituted GEN-1 (in the 50 mL glass vial or IV bag) is stable at room temperature for up to 24 hours. Upon confirmation of catheter patency, an IV bag containing the GEN-1 will be administered through the patient's IP catheter. GEN-1 will be infused via a catheter through gravity from the IV bag with the valve all the way open and free flowing. Typical administrations may take about 1 hour.

Following the GEN-1 infusion, a second flush of at least 25 ml 0.9% Normal Saline for Injection to ensure study drug is cleared from the catheter.

### Intraperitoneal Catheter

Subcutaneously implantable IP silicone catheters may be used to administer GEN-1 to the peritoneal cavity. GEN-1 has been demonstrated to be compatible with silicone catheters in a prior preclinical compatibility study and in prior phase I studies. Port-A-Cath catheter (Deltec, Inc. St. Paul, MN) has been successfully used for IP delivery of GEN-1 with little to no catheter-related serious complications noted. Any other approved catheter with a subcutaneous port for IP delivery may also be used if suitable for aspiration of biological samples for translational studies. Bard 9.6 Fr silicone single lumen catheters for venous access or the equivalent with or without cuff from Bard Access Systems (West Amelia Earhart Drive Salt Lake City Utah) may be used. The catheter compatibility studies conducted with Port-A-Cath catheter showed that catheter exposure to GEN-1 does not significantly affect the physico-chemical properties or transfection activity of GEN-1. A similar compatibility study was performed with a Bard catheter. Subjects may be treated via pre-existing IP catheters provided they are of a similar nature to a Port-A-Cath catheter device and functional. A cathetergram may be obtained to verify intraabdominal infusion if there is concern about catheter patency or function.

### Catheter Insertion

The IP catheter will be implanted per hospital standard process; the procedure and risks associated with placement of the IP catheter must be explained to the subject and an operative consent form will be obtained from the subject prior to placement of the catheter. The subject will undergo insertion of the IP catheter a minimum of 7 days prior to scheduled study drug administration to allow for sufficient healing and sealing around the catheter site. A semi-permanent subcutaneous access port, such as the Port-A-Cath catheter, (SIMS Deltec, Inc, St. Paul MN Inc., 55112) or equivalent device per current institutional clinical practice will be used.

Study drug will be infused through this port during the course of the study. Prior to each infusion of study drug, approximately 25 mL of saline will be flushed through the catheter to check for catheter patency; heparin should not be used to flush the catheter at the time of sample collection or drug infusion. At end of study, the catheter may be removed at completion of GEN-1 administration at the clinician's discretion.

Subjects should be monitored for study-drug related toxicity.

### Weight Change

The calculated dose of GEN-1 is based on the protocol specified dose (in mg/m²) and the subject's body BSA calculated at baseline. The subject's weight should be monitored during treatment (e.g., typically weight is monitored/recorded on the same day of treatment). If the subject's weight changes by >10%, then the BSA is recalculated and the adjusted dose will be administered during the subsequent treatment cycles. The new BSA becomes the baseline for future weight and any dose modifications.

### Dose Modifications for Dual ICI (See Nivolumab Prescribing Information)

When nivolumab is administered in combination with ipilimumab, withhold or permanently discontinue both ipilimumab and nivolumab for an adverse reaction meeting these dose modification guidelines.

### ICI and GEN-1 Treatment

Each cycle of study treatment will be defined as being 6 weeks. On day 1 of each cycle all subjects will receive a fixed dose of the dual ICI (nivolumab and ipilimumab) followed by the prescribed dose of Gen-1. Dosing by cycle is presented in the table below:

TABLE 5
Treatment Cycle
Treatment Cycle (6 weeks)

| Day | 1 | 15 | 28 |
|---|---|---|---|
| Ipilimumab | X | | |
| Nivolumab | X | X | X |
| Gen-1 | X | X | X |

### Safety

Subjects will be monitored for safety (with physical exams and assessment of AEs) at every treatment visit from the time of signing informed consent until at least 30 days following their last dose of study drug. Any suspected drug related adverse events may be reported at any time during follow up until resolution to a grade≤2 (CTCAE v5.0).

### Assessment Of Efficacy

The primary end point is objective tumor response (complete or partial) by RECIST, version 1.1,16 prior to progression will be used as a method to evaluate efficacy.

### Disease Parameters

Measurable disease. Measurable lesions are defined as those that can be accurately measured in at least one dimension (longest diameter to be recorded) as ≥20 mm by chest x-ray, as ≥10 mm with CT scan, or ≥10 mm with calipers by clinical exam. All tumor measurements must be recorded in millimeters (or decimal fractions of centimeters).

Note: Tumor lesions that are situated in a previously irradiated area will not be considered measurable unless progression is documented, or a biopsy is obtained to confirm persistence at least 90 days following completion of radiation therapy.

Malignant lymph nodes. To be considered pathologically enlarged and measurable, a lymph node must be ≥15 mm in short axis when assessed by CT scan (CT scan slice thickness recommended to be no greater than 5 mm). At baseline and in follow-up, only the short axis will be measured and followed.

Non-measurable disease. All other lesions (or sites of disease), including small lesions (longest diameter <10 mm or pathological lymph nodes with ≥10 to <15 mm short axis), are considered non-measurable disease. Leptomeningeal disease, ascites, pleural/pericardial effusions, lymphangitis cutis/pulmonitis, inflammatory breast disease, and abdominal masses (identified by physical exam and not CT or MRI), are considered as non-measurable.

Bone lesions: Lytic bone lesions or mixed lytic-blastic lesions, with identifiable soft tissue components, that can be evaluated by CT or MRI can be considered as measurable lesions if the soft tissue component meets the definition of measurability described above. Blastic bone lesions are non-measurable.

Cystic lesions that meet the criteria for radiographically defined simple cysts should not be considered as malignant lesions (neither measurable nor non-measurable) since they are, by definition, simple cysts. ‘Cystic lesions’ thought to represent cystic metastases can be considered as measurable lesions, if they meet the definition of measurability described above. However, if non-cystic lesions are present in the same patient, these are preferred for selection as target lesions.

Target lesions. All measurable lesions up to a maximum of 2 lesions per organ and 5 lesions in total, representative of all involved organs, should be identified as target lesions and recorded and measured at baseline. Target lesions should be selected on the basis of their size (lesions with the longest diameter), be representative of all involved organs, but in addition should be those that lend themselves to reproducible repeated measurements. It may be the case that, on occasion, the largest lesion does not lend itself to reproducible measurement in which circumstance the next largest lesion which can be measured reproducibly should be selected. A sum of the diameters (longest for non-nodal lesions, short axis for nodal lesions) for all target lesions will be calculated and reported as the baseline sum diameters. If lymph nodes are to be included in the sum, then only the short axis is added into the sum. The baseline sum diameters will be used as reference to further characterize any objective tumor regression in the measurable dimension of the disease.

Non-target lesions. All other lesions (or sites of disease) including any measurable lesions over and above the 5 target lesions should be identified as non-target lesions and should also be recorded at baseline. Measurements of these lesions are not required, but the presence, absence, or in rare cases unequivocal progression of each should be noted throughout follow-up.

### Study Endpoints

### Primary Endpoint: Objective Response Rate

Phase I: Safety (DLT's and MTD), 3+3 design in 12 to 18 patients.

Phase II: ORR (proportion of subjects achieving CR or PR. The same analysis methodology as with the primary endpoint will be used) will be assessed in two stage design. N=40.

What is claimed is:

1. A combination therapy comprising:
(a) a nucleic acid vector comprising a polynucleotide that encodes an interleukin-12 (IL-12) formulated with a lipopolymer; and
(b) an immune checkpoint inhibitor.

2. The combination therapy of claim 1, wherein the polynucleotide encodes human IL-12.

3. The combination therapy of claim 1 or 2, wherein the nucleic acid vector comprises a promoter operably linked to a nucleic acid encoding a p35 subunit of IL-12 and a promoter operably linked to a nucleic acid encoding a p40 subunit of IL-12.

4. The combination therapy of any one of claims 1-3, wherein the nucleic acid comprises an intron, a 3′ UTR, an antibiotic resistance gene, or any combination thereof (e.g., the elements of FIG. 1).

5. The combination therapy of any one of claims 1-4, wherein the lipopolymer comprises a polyethyleneimine (PEI) covalently linked independently to cholesterol and polyethylene glycol (PEG) groups (e.g., the lipopolymer of FIG. 2).

6. The combination therapy of any one of claims 1-5, wherein the combination further comprises an anticancer agent.

7. The combination therapy of claim 6, wherein the anticancer agent is a chemotherapeutic agent.

8. The combination therapy of claim 6, wherein the anticancer agent is selected from the group consisting of doxorubicin, paclitaxel, carboplatin, docetaxel, nab-paclitaxel, olaparib, and any combination thereof.

9. The combination therapy of claim 6, wherein the anticancer agent is paclitaxel.

10. The combination therapy of claim 6, wherein the anticancer agent is carboplatin.

11. The combination therapy of claim 6, wherein the anticancer agent is docetaxel.

12. The combination therapy of claim 6, wherein the anticancer agent is nab-paclitaxel.

13. The combination therapy of claim 6, wherein the anticancer agent is olaparib.

14. The combination therapy of any one of claims 1-13, wherein the immune checkpoint inhibitor is an inhibitor of an immune checkpoint protein selected from the group consisting of CTLA-4, PD-1 (and its ligands PD-L1 and PD-L2), LAG-3, and any combination thereof.

15. The combination therapy of any one of claims 1-14, wherein the immune checkpoint inhibitor is an antibody.

16. The combination therapy of any one of claims 1-14, wherein the immune checkpoint inhibitor comprises an anti-CTLA4 antibody, an anti-PD-1 antibody, an anti-PD-L1 antibody, or any combination thereof.

17. The combination therapy of any one of claims 1-15, wherein the immune checkpoint inhibitor is a PD-1 antagonist selected from the group consisting of nivolumab, pembrolizumab, dostarlimab, cemiplimab, and any combination thereof.

18. The combination therapy of claim 17, wherein the PD-1 antagonist is nivolumab.

19. The combination therapy of any one of claims 1-15, wherein the immune checkpoint inhibitor is a PD-L1 antagonist selected from the group consisting of atezolizumab, durvalumab, and avelumab.

20. The combination therapy of any one of claims 1-15, wherein the immune checkpoint inhibitor is a CTLA-4 antagonist is ipilimumab.

21. The combination therapy of any one of claims 1-15, wherein the immune checkpoint inhibitor is a LAG-3 antagonist, wherein the LAG-3 antagonist is relatlimab.

22. The combination therapy of any one of claims 1-21, wherein the combination therapy comprises two or more immune checkpoint inhibitors.

23. The combination therapy of claim 22, wherein the combination therapy comprises two, three, or four immune checkpoint inhibitors.

24. The combination therapy of claim 23, wherein the immune checkpoint inhibitor comprises two or more inhibitors of an immune checkpoint protein selected from the group consisting of CTLA-4, PD-1 (and its ligands PD-L1 and PD-L2), and LAG-3.

25. The combination therapy of claim 22 or 24, wherein the second of the two or more checkpoint inhibitors are antibodies.

26. The combination therapy of claim 22 or 24, wherein at least one immune checkpoint inhibitor is a small molecule inhibitor.

27. The combination therapy of any one of claims 22-25, wherein the immune checkpoint inhibitor comprises a PD-1 antagonist selected from the group consisting of nivolumab, pembrolizumab, dostarlimab, and cemiplimab.

28. The combination therapy of claim 27, wherein the checkpoint inhibitor comprises nivolumab.

29. The combination therapy of any one of claims 22-25, wherein the checkpoint inhibitor comprises a PD-L1 antagonist selected from the group consisting of atezolizumab, durvalumab, and avelumab.

30. The combination therapy of any one of claims 22-25, wherein the immune checkpoint inhibitor comprises a CTLA-4 antagonist.

31. The combination therapy of claim 30, wherein the CTLA-4 antagonist is ipilimumab.

32. The combination therapy of any one of claims 22-25, wherein the immune checkpoint inhibitor comprises a LAG-3 antagonist.

33. The combination therapy of claim 32, wherein the LAG-3 antagonist is relatlimab.

34. The combination therapy of any one of claims 4-33, wherein the 3′ UTR is an hGH 3′ UTR.

35. The combination therapy of any one of claims 1-34, wherein the nucleic acid vector is a plasmid.

36. The combination therapy of any one of claims 1-35, wherein the lipopolymer is a nanoparticle.

37. A method of treating a subject suffering from cancer comprising administering to the subject the combination therapy of any one of claims 1-36.

38. The method of claim 37, wherein the nucleic acid vector formulated with the lipopolymer is administered intratumorally or intraperitoneally.

39. The method of claim 37 or 38, wherein the nucleic acid vector formulated with the lipopolymer is administered intravenously.

40. The method of any one of claims 37-39, wherein the immune checkpoint inhibitor is administered intratumorally, intraperitoneally, intravenously, intravesicularly, or any combination thereof.

41. The method of any one of claims 37-40, wherein the immune checkpoint inhibitor is administered intravenously.

42. The method of any one of claims 37-41, wherein the nucleic acid vector formulated with the lipopolymer is administered prior to, concurrently with, or after the immune checkpoint inhibitor.

43. The method of any one of claims 37-42, wherein the nucleic acid vector formulated with the lipopolymer is administered prior to, concurrently with, or after the anticancer agent.

44. The method of any one of claims 37-43, wherein an anticancer agent is administered, followed by the administration of the nucleic acid vector formulated with the lipopolymer, and followed by administration of the immune checkpoint inhibitor.

45. The method of any one of claims 37-43, wherein an anticancer agent is administered, followed by the administration of the nucleic acid vector formulated with the lipopolymer, followed by one or more immune checkpoint inhibitors, and followed by a surgery to remove all or part of a tissue or tumor.

46. The method of any one of claims 37-43, wherein a surgery to remove all or part of a tissue or tumor is administered, followed by the administration of the nucleic acid vector formulated with the lipopolymer is administered, and followed by administration of the immune checkpoint inhibitor.

47. The method of any one of claims 45-46, wherein the surgery is interval cytoreductive surgery.

48. The method of any one of claims 37-43, wherein the anticancer agent is administered, followed by the administration of the DNA nucleic acid vector, followed by the immune checkpoint inhibitor, optionally, followed by an interval cytoreductive surgery.

49. The method of any one of claims 37-48, wherein the administration of the anticancer agent comprises administering paclitaxel at a dose of about 25-250 mg/m², optionally, followed by administering carboplatin at a dose of about AUC 4-6 IV.

50. The method of any one of claims 37-48, wherein the administration of the anticancer agent comprises administering docetaxel at a dose of 25-250 mg/m², optionally, followed by administering carboplatin at a dose of about AUC 4-6 IV.

51. The method of any one of claims 37-48, wherein the administration of the anticancer agent comprises administering nab-paclitaxel at a dose of 25-350 mg/m², optionally followed by administering carboplatin at a dose of about AUC 4-6 IV.

52. The method of any one of claims 37-51, wherein the nucleic acid vector comprising a polynucleotide encoding interleukin-12 (IL-12) formulated with a lipopolymer is administered at a dose of about 35 mg/m² to about 80 mg/m².

53. The method of any one of claims 37-52, wherein the cancer is selected from a group consisting of ovarian cancer, fallopian tube cancer, primary peritoneal cancer, cervical cancer, breast cancer, prostate cancer, colorectal cancer, bladder cancer, brain cancer, lung cancer, any combination thereof, and metastasis of any of the cancers.

54. The method of claim 53, wherein the brain cancer is a glioblastoma.

55. The method of any one of claims 37-54, wherein the cancer is selected from a group consisting of ovarian cancer, fallopian tube cancer, primary peritoneal cancer, and any combination thereof.

56. The method of any one of claims 37-55, wherein the subject is a human.

57. The method of any one of claims 37-56, wherein the lipopolymer is administered at a dose of about 60 mg/m².

58. The method of any one of claims 37-57, wherein the immune checkpoint inhibitor comprises nivolumab, optionally, wherein nivolumab is administered at about 240 mg.

59. The method of claim 58, wherein nivolumab and the lipopolymer are administered every 1-4 weeks during the treatment.

60. The method of claim 59, wherein nivolumab and the lipopolymer are administered every 2 weeks during the treatment.

61. The method of claim any one of claims 37-60, wherein the second inhibitor is ipilimumab, optionally, wherein ipilimumab is administered at about 1 mg/kg.

62. The method of claim 61, wherein ipilimumab is administered every 2-4 weeks during the treatment.

63. The method of claim 62, wherein ipilimumab is administered every 6 weeks during the treatment.

64. The method of any one of claims 37-63, wherein the nucleic acid vector is a plasmid.

65. The method of any one of claims 37-64, wherein the lipopolymer is a nanoparticle.
